(19) United States
(12) Patent Application Publication
(10) Pub. No.: US 2024/0097194 A1
(43) Pub. Date: Mar. 21, 2024

(54) IONIC LIQUID, SECONDARY BATTERY, ELECTRONIC DEVICE, AND VEHICLE

(71) Applicant: Semiconductor Energy Laboratory Co., Ltd., Atsugi-shi, Kanagawa-ken (JP)

(72) Inventors: Kaori OGITA, Isehara (JP); Kazuya SHIMADA, Atsugi (JP); Takashi HIRAHARA, Atsugi (JP); Fumiko TANAKA, Atsugi (JP); Shotaro MURATSUBAKI, Atsugi (JP); Satoshi SEO, Sagamihara (JP)

(21) Appl. No.: 18/039,505

(22) PCT Filed: Dec. 3, 2021
(86) PCT No.: PCT/IB2021/061274

§ 371 Date: May 31, 2023

(30) Foreign Application Priority Data
Dec. 16, 2020 (JP) 2020-208710
Dec. 16, 2020 (JP) 2020-208711

(51) Int. Cl. H01M 10/0569; C01G 53/00; H01M 4/525; H01M 10/0525; H01M 10/0567

(52) U.S. Cl. CPC H01M 10/0569; C01G 53/66; H01M 4/525; H01M 10/0525; H01M 10/0567; C01P 2002/72; C01P 2002/76; C01P 2006/40; H01M 2004/028

(57) ABSTRACT

A novel ionic liquid is provided. A highly safe secondary battery with high charge and discharge capacity is provided. The ionic liquid includes a cation represented by General Formula (G1) and an anion represented by Structural Formula (200). In the formula, X¹ to X³ each independently represent any one of fluorine, chlorine, bromine, and iodine. One of X¹ to X³ may be hydrogen. In addition, n and m each independently represent 0 to 5. Furthermore, a secondary battery including the above-described ionic liquid is provided.

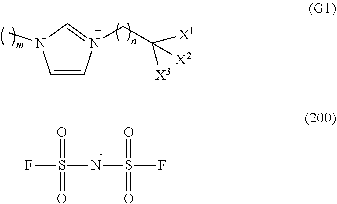

### TECHNICAL FIELD

One embodiment of the present invention relates to an ionic liquid, a secondary battery, an electronic device, and a vehicle.

One embodiment of the present invention relates to an object, a method, or a manufacturing method. Alternatively, the present invention relates to a process, a machine, manufacture, or a composition of matter. One embodiment of the present invention relates to a semiconductor device, a display device, a light-emitting device, a power storage device, a lighting device, an electronic device, or a manufacturing method thereof.

Note that electronic devices in this specification mean all devices including power storage devices, and electro-optical devices including power storage devices, information terminal devices including power storage devices, and the like are all electronic devices.

Note that in this specification, a power storage device refers to every element and device having a function of storing power. For example, a power storage device (also referred to as a secondary battery) such as a lithium-ion secondary battery, a lithium-ion capacitor, and an electric double layer capacitor are included.

### BACKGROUND ART

In recent years, a variety of power storage devices such as lithium-ion secondary batteries, lithium-ion capacitors, and air batteries have been actively developed. In particular, demand for lithium-ion secondary batteries with high output and high energy density has rapidly grown with the development of the semiconductor industry, for portable information terminals such as mobile phones, smartphones, and laptop computers, portable music players, digital cameras, medical equipment, next-generation clean energy vehicles such as hybrid electric vehicles (HVs), electric vehicles (EVs), and plug-in hybrid electric vehicles (PHVs), and the like, and the lithium-ion secondary batteries are essential as rechargeable energy supply sources for today's information society.

As described above, lithium-ion secondary batteries have been used for a variety of purposes in various fields. The performance required for lithium-ion secondary batteries includes high energy density, excellent charge and discharge cycle performance, and safety in a variety of operation environments.

Many of the widely used lithium-ion secondary batteries include a nonaqueous electrolyte (also referred to as electrolyte solution) including a nonaqueous solvent and a lithium salt containing lithium ions. An example of an organic solvent often used in the nonaqueous electrolyte is an organic solvent having a high dielectric constant and excellent ionic conductivity, such as ethylene carbonate.

However, the above organic solvent has volatility and a low flash point; thus, when the organic solvent is used in a lithium-ion secondary battery, an increase in the internal temperature of the lithium-ion secondary battery due to an internal short circuit, overcharging, or the like might cause the lithium-ion secondary battery to explode or catch fire, for example.

In view of the above, an ionic liquid (also referred to as a room temperature molten salt) with non-flammability and non-volatility has been proposed to be used as a solvent of a nonaqueous electrolyte of a lithium-ion secondary battery. Examples the ionic liquid include an ionic liquid containing an ethylmethylimidazolium (EMI) cation and an ionic liquid containing a 1-ethyl-2,3-dimethylimidazolium cation (2MeEMI) (Patent Document 1).

Meanwhile, study on the crystal structures of positive electrode active materials has also been conducted to improve energy density, charge and discharge cycle performance, and the like (Non-Patent Document 1 to Non-Patent Document 3). X-ray diffraction (XRD) is one of methods used for analysis of the crystal structure of a positive electrode active material. With the use of ICSD (Inorganic Crystal Structure Database) described in Non-Patent Document 3, XRD data can be analyzed.

### REFERENCE

### Patent Document

[Patent Document 1] Japanese Published Patent Application No. 2016-096023

### Non-Patent Document

[Non-Patent Document 1] Motohashi, T. et al., “Electronic phase diagram of the layered cobalt oxide system LixCoO₂ (0.0≤x≤1.0)”, Physical Review B, 80 (16); 165114.
[Non-Patent Document 2] Zhaohui Chen et al., “Staging Phase Transitions in LixCoO₂”, Journal of The Electrochemical Society, 2002, 149 (12), A1604-A1609.
[Non-Patent Document 3] Belsky, A. et al., “New developments in the Inorganic Crystal Structure Database (ICSD): accessibility in support of materials research and design”, Acta Cryst., (2002) B58, pp. 364-369.

### SUMMARY OF THE INVENTION

### Problems to be Solved by the Invention

Development of lithium-ion secondary batteries is susceptible to improvement in terms of charge and discharge characteristics, cycle performance, reliability, safety, cost, and other various aspects.

An object of one embodiment of the present invention is to provide a novel ionic liquid that can be used for a lithium-ion secondary battery. Another object of one embodiment of the present invention is to provide a secondary battery with high charge and discharge capacity. Another object of one embodiment of the present invention is to provide a secondary battery with excellent cycle performance. Another object of one embodiment of the present invention is to provide a highly safe secondary battery. Another object of one embodiment of the present invention is to provide a secondary battery with reduced irreversible capacity. Another object of one embodiment of the present invention is to provide a highly reliable secondary battery. Another object of one embodiment of the present invention is to provide a long-life secondary battery.

Another object of one embodiment of the present invention is to provide a secondary battery that can be used in a wide temperature range. Another object of one embodiment of the present invention is to provide a high-performance secondary battery. Another object of one embodiment of the present invention is to provide a novel secondary battery.

Note that the description of these objects does not preclude the existence of other objects. One embodiment of the present invention does not have to achieve all the objects. Note that other objects will be apparent from the description of the specification, the drawings, the claims, and the like, and other objects can be derived from the description of the specification, the drawings, the claims, and the like.

### Means for Solving the Problems

One embodiment of the present invention is an ionic liquid including a cation represented by General Formula (G1) and an anion represented by Structural Formula (200).

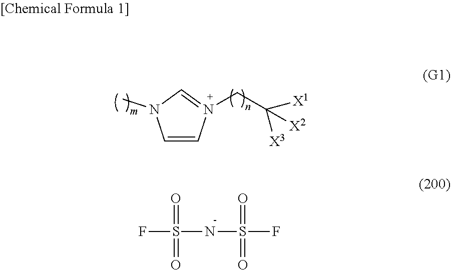

In the formula, X¹ to X³ each independently represent any one of fluorine, chlorine, bromine, and iodine. One of X¹ to X³ may be hydrogen. In addition, n and m each independently represent 0 to 5.

One embodiment of the present invention is an ionic liquid including a cation represented by Structural Formula (100) and an anion represented by Structural Formula (200).

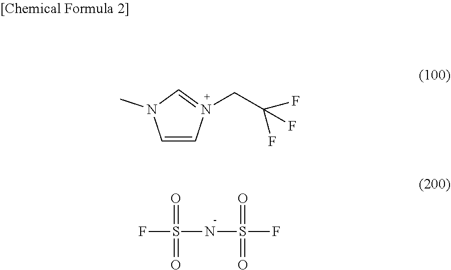

One embodiment of the present invention is an ionic liquid including a cation represented by Structural Formula (150) and an anion represented by Structural Formula (200).

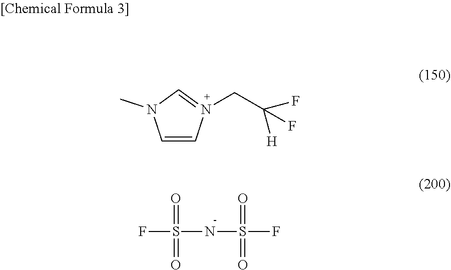

Another embodiment of the present invention is a secondary battery including a positive electrode, a negative electrode, and an electrolyte, and the electrolyte includes the ionic liquid described above.

It is preferable that in the above, the electrolyte further include an additive agent and the additive agent be at least one of succinonitrile, adiponitrile, fluoroethylene carbonate, and propane sultone.

It is preferable that in the above, the positive electrode include a positive electrode active material; the positive electrode active material be lithium cobalt oxide to which magnesium, fluorine, aluminum, and nickel are added; and an XRD pattern at least have a diffraction peak at 2θ=19.30±0.20° and 2θ=45.55±0.10° when the positive electrode is analyzed by powder X-ray diffraction with CuKα₁ radiation in an argon atmosphere in the following manner: the positive electrode and a counter electrode of a lithium metal are used, a mixture in which 2 wt % of vinylene carbonate is added to lithium hexafluorophosphate, ethylene carbonate, and diethyl carbonate is used as the electrolyte, constant current charge with a current value of 0.5 C (note that 1 C=137 mA/g) is performed to a voltage of 4.6 V in an environment at 25° C., and then constant voltage charge is performed until the current value becomes 0.01 C.

It is preferable that in the above, the diffusion state of each of magnesium and aluminum included in the positive electrode active material vary on each crystal plane of a surface portion.

It is preferable that in the above, the positive electrode active material have a crystal structure belonging to the space group R-3m, and magnesium and aluminum exist in a deeper position of the surface portion in a region with a crystal plane other than (001) than in a region with the crystal plane (001).

Another embodiment of the present invention is an electronic device including the secondary battery described above, and at least one of a display device, an operation button, an external connection port, a speaker, and a microphone.

Another embodiment of the present invention is a vehicle including the secondary battery described above, and at least one of a motor, a brake, and a control circuit.

### Effect of the Invention

One embodiment of the present invention can provide a novel ionic liquid that can be used for a lithium-ion secondary battery. Another embodiment of the present invention can provide a secondary battery with high charge and discharge capacity. Another embodiment of the present invention can provide a secondary battery with excellent cycle performance. Another embodiment of the present invention can provide a highly safe secondary battery. Another embodiment of the present invention can reduce the irreversible capacity of a secondary battery. Another embodiment of the present invention can provide a highly reliable power storage device. Another embodiment of the present invention can provide a long-life secondary battery.

Another embodiment of the present invention can provide a secondary battery that can be used in a wide temperature range. Another embodiment of the present invention can provide a high-performance secondary battery. Another embodiment of the present invention can provide a novel secondary battery.

Note that the description of these effects does not preclude the existence of other effects. One embodiment of the present invention does not need to have all of these effects. Note that other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A is a top view of a secondary battery, and FIG. 1B is a cross-sectional view of the secondary battery.

FIG. 2A is a cross-sectional view of a positive electrode active material, and FIG. 2B1 to FIG. 2C2 are parts of a cross-sectional view of the positive electrode active material.

FIG. 3A and FIG. 3B are cross-sectional views of a positive electrode active material, and FIG. 3C1 and FIG. 3C2 are parts of a cross-sectional view of the positive electrode active material.

FIG. 4 is a cross-sectional view of a positive electrode active material.

FIG. 5 is a cross-sectional view of a positive electrode active material.

FIG. 6 is a diagram illustrating the charge depth and crystal structures of a positive electrode active material.

FIG. 7 shows XRD patterns calculated from crystal structures.

FIG. 8 is a diagram illustrating the charge depth and crystal structures of a positive electrode active material of a comparative example.

FIG. 9 shows XRD patterns calculated from crystal structures.

FIG. 10A to FIG. 10C show lattice constants calculated using XRD.

FIG. 11A to FIG. 11C show lattice constants calculated using XRD.

FIG. 12 is an example of a TEM image showing crystal orientations substantially aligned with each other.

FIG. 13A is an example of a STEM image showing crystal orientations substantially aligned with each other. FIG. 13B shows an FFT pattern in a rock-salt crystal RS region, and FIG. 13C is an FFT pattern in a layered rock-salt crystal LRS region.

FIG. 14A to FIG. 14C show a method for forming a positive electrode active material.

FIG. 15A and FIG. 15B are cross-sectional views of an active material layer using a graphene compound as a conductive material.

FIG. 16A and FIG. 16B are diagrams illustrating a coin-type secondary battery. FIG. 16C is a diagram for showing charge and discharge of a secondary battery.

FIG. 17A to FIG. 17D are diagrams illustrating cylindrical secondary batteries.

FIG. 18A and FIG. 18B are diagrams illustrating an example of a secondary battery.

FIG. 19A to FIG. 19D are diagrams illustrating examples of a secondary battery.

FIG. 20A and FIG. 20B are diagrams illustrating examples of a secondary battery.

FIG. 21 is a diagram illustrating an example of a secondary battery.

FIG. 22A to FIG. 22C are diagrams illustrating a laminated secondary battery.

FIG. 23A and FIG. 23B are diagrams illustrating a laminated secondary battery.

FIG. 24A and FIG. 24B are external views of a secondary battery.

FIG. 25A to FIG. 25C are diagrams illustrating a method for manufacturing a secondary battery.

FIG. 26A to FIG. 26H are diagrams illustrating examples of electronic devices.

FIG. 27A to FIG. 27C are diagrams illustrating examples of an electronic device.

FIG. 28 is a diagram illustrating examples of electronic devices.

FIG. 29A to FIG. 29D are diagrams illustrating examples of electronic devices.

FIG. 30A to FIG. 30C are diagrams illustrating examples of electronic devices.

FIG. 31A to FIG. 31C are diagrams illustrating examples of vehicles.

FIG. 32 is a ¹H-NMR chart of F3EMI-FSI.

FIG. 33 is a ¹⁹F-NMR chart of F3EMI-FSI.

FIG. 34A is a cyclic voltammogram of F3EMI-FSI and FIG. 34B is a cyclic voltammogram of EMI-FSI.

FIG. 35 is a charge and discharge curve of a secondary battery including F3EMI-FSI.

FIG. 36 is a ¹H-NMR chart of F2EMI-TfO.

FIG. 37A is a ¹H-NMR chart of F2EMI-FSI. FIG. 37B is a ¹⁹F-NMR chart of F2EMI-FSI.

FIG. 38A is a cyclic voltammogram of F2EMI-FSI and FIG. 38B is a cyclic voltammogram of EMI-FSI.

FIG. 39 is a charge and discharge curve of a secondary battery including F2EMI-FSI.

### MODE FOR CARRYING OUT THE INVENTION

Embodiment is described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Note that in structures of the invention described below, the same portions or portions having similar functions are denoted by the same reference numerals in different drawings, and the description thereof is not repeated. Furthermore, the same hatch pattern is used for the portions having similar functions, and the portions are not especially denoted by reference numerals in some cases.

Note that the position, size, range, or the like of each structure illustrated in drawings and the like is not accurately represented in some cases for easy understanding. Therefore, the disclosed invention is not necessarily limited to the position, size, range, and the like disclosed in the drawings and the like.

Note that the ordinal numbers such as “first” and “second” in this specification and the like are used for convenience and do not denote the order of steps, the stacking order of layers, and the like. Therefore, for example, description can be made even when “first” is replaced with “second”, “third”, or the like as appropriate. In addition, the ordinal numbers in this specification and the like do not sometimes correspond to the ordinal numbers that are used to specify one embodiment of the present invention.

Note that in this specification and the like, a positive electrode and a negative electrode for a power storage device may be collectively referred to as an electrode; in this case, the electrode refers to at least one of the positive electrode and the negative electrode.

Here, a charge rate and a discharge rate are described. For example, in the case where constant current charge is performed on a storage battery with a capacity of X [Ah], a charge rate 1 C means a current value I [A] with which charge is terminated in exactly 1 hour, and a charge rate 0.2 C means I/5 [A] (i.e., a current value with which charge is terminated in exactly 5 hours). Similarly, a discharge rate 1 C means the current value I [A] with which discharge is terminated in exactly 1 hour; for example, a discharge rate 0.2 C means I/5 [A] (i.e., the current value with which discharge is terminated in exactly 5 hours).

Here, an active material refers only to a material that is involved in insertion and extraction of ions that are carriers; in this specification and the like, a layer including an active material is referred to as an active material layer. The active material layer may include a conductive additive and a binder in addition to the active material.

In this specification and the like, the Miller index is used for the expression of crystal planes and crystal orientations. An individual plane that shows a crystal plane is denoted by “( )”. In the crystallography, a bar is placed over a number in the expression of crystal planes, crystal orientations, and space groups; in this specification and the like, because of format limitations, crystal planes, crystal orientations, and space groups are sometimes expressed by placing a minus sign (−) in front of a number instead of placing a bar over the number. Furthermore, an individual direction which shows an orientation in a crystal is denoted with “[ ]”, a set direction which shows all of the equivalent orientations is denoted with “< >”, an individual plane which shows a crystal plane is denoted with “( )”, and a set plane having equivalent symmetry is denoted with “{ }”. As the Miller indices of trigonal system and hexagonal system such as R-3m, not only (hkl) but also (hkil) is used in some cases. Here, i is −(h+k).

In this specification and the like, the theoretical capacity of a positive electrode active material refers to the amount of electricity for the case where all the lithium that can be inserted and extracted in the positive electrode active material is extracted. For example, the theoretical capacity of LiCoO₂ is 274 mAh/g, the theoretical capacity of LiNiO₂ is 274 mAh/g, and the theoretical capacity of LiMn₂O₄ is 148 mAh/g. In this specification and the like, a charge depth is used as an indicator; the charge depth obtained when all the lithium that can be inserted and extracted is inserted is 0, and the charge depth obtained when all the lithium that can be inserted and extracted and is contained in a positive electrode active material is extracted is 1.

### Embodiment 1

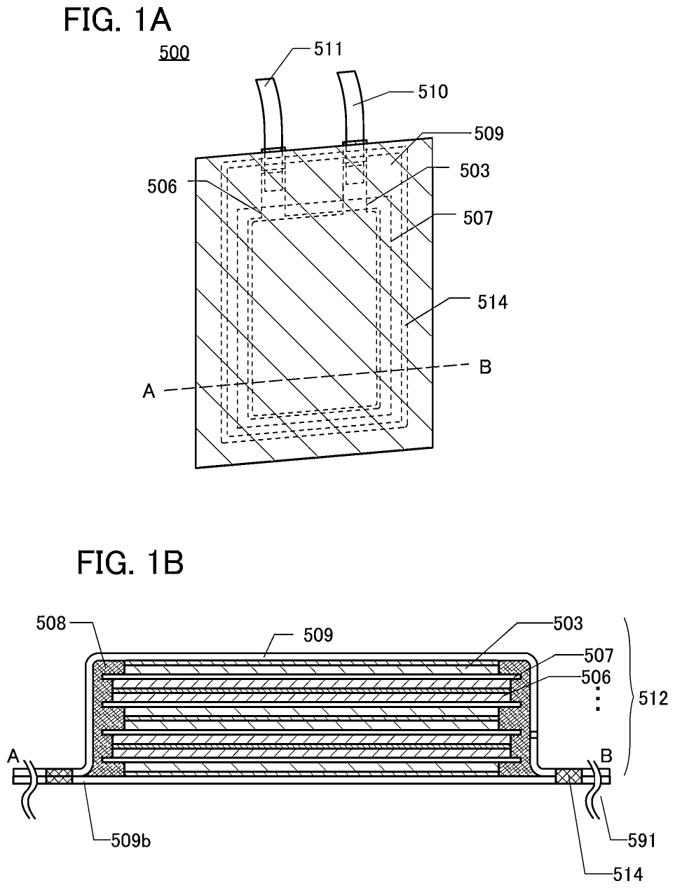

In this embodiment, an electrolyte included in a secondary battery of one embodiment of the present invention will be described with reference to FIG. 1A and FIG. 1B.

Although a lithium-ion secondary battery is described as an example in this embodiment, a power storage device of one embodiment of the present invention is not limited to this. One embodiment of the present invention can also be used not only for the lithium-ion secondary battery but also for a variety of primary batteries and secondary batteries such as a lithium air battery, a lead storage battery, a lithium-ion polymer secondary battery, a nickel-hydrogen storage battery, a nickel-cadmium storage battery, a nickel-iron storage battery, a nickel-zinc storage battery, a silver oxide-zinc storage battery, a solid-state battery, and an air battery, a capacitor, a lithium-ion capacitor, and the like.

FIG. 1A is a top view of a secondary battery 500 of one embodiment of the present invention. FIG. 1B is a cross-sectional view along dashed-dotted line AB in FIG. 1A.

The secondary battery 500 includes an exterior body 509, a positive electrode 503, a negative electrode 506, a separator 507, and an electrolyte 508. The separator 507 is provided between the positive electrode 503 and the negative electrode 506. The exterior body 509 is filled with the electrolyte 508. The exterior body 509 is sealed in a region 514. The secondary battery 500 may include a positive electrode lead electrode 510 and a negative electrode lead electrode 511.

The positive electrode 503 includes a positive electrode active material layer and a positive electrode current collector. The negative electrode 506 includes a negative electrode active material layer and a negative electrode current collector. The active material layer is formed on one surface or both surfaces of the current collector.

FIG. 1A and FIG. 1B illustrate an example of the secondary battery 500 including a stack 512 in which a plurality of positive electrodes 503 and a plurality of negative electrodes 506 are stacked; however, the present invention is not limited thereto. The number of positive electrodes and the number of negative electrodes may each be one or more.

The electrolyte 508 of the secondary battery of one embodiment of the present invention includes a lithium salt and an ionic liquid. The ionic liquid includes one or more kinds of cations and one or more kinds of anions.

The ionic liquid of one embodiment of the present invention includes an organic compound represented by General Formula (G1) below as a cation. The ionic liquid also includes bis(fluorosulfonyl)imide (FSI) represented by Structural Formula (200) below as an anion.

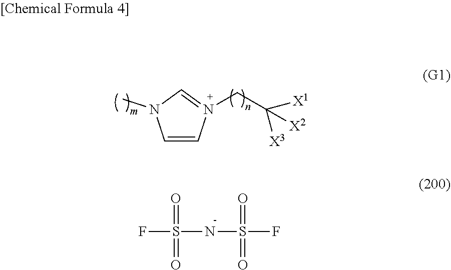

Next, a specific structural formula of the ionic liquid of one embodiment of the present invention is shown below. The ionic liquid of one embodiment of the present invention includes 1-methyl-3-(2,2,2-trifluoroethyl)-imidazolium (F3EMI) represented by Structural Formula (100) as a cation. The ionic liquid also includes bis(fluorosulfonyl)imide (FSI) represented by Structural Formula (200) as an anion.

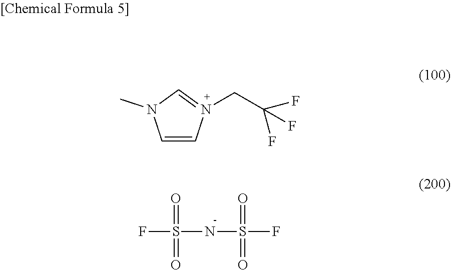

Alternatively, the ionic liquid of one embodiment of the present invention includes 1-(2,2-difluoroethyl)-3-methyl-imidazolium (F2EMI) represented by Structural Formula (150) as a cation. The ionic liquid also includes bis(fluorosulfonyl)imide (FSI) represented by Structural Formula (200) as an anion. Note that the present invention is not limited thereto.

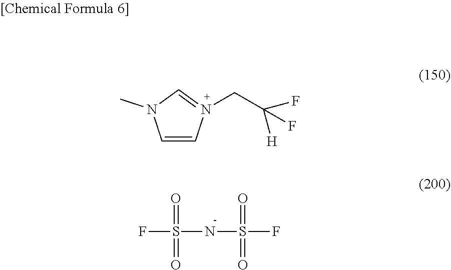

These ionic liquids in which lithium salts are dissolved each function as an electrolyte capable of transferring lithium ions. In the ionic liquid, lithium ions are solvated by anions of the ionic liquid.

In these ionic liquids, terminals of cations are substituted by fluorine; thus, the HOMO (Highest Occupied Molecular Orbital) levels can be lowered to increase the oxidation resistance of the ionic liquids. In addition, the stability of the ionic liquids is improved because of their high bond energy of a C—F bond. This is preferable because the ionic liquids are unlikely to be decomposed even when used in a secondary battery that is repeatedly charged at a high voltage that is higher than the oxidation-reduction potential of a lithium metal by higher than or equal to 4.6 V. Furthermore, the ionic liquids have flame resistance, so that the electrolyte including the ionic liquids can increase the safety of the secondary battery.

Next, specific examples of the organic compound represented by General Formula (G1) above, which are other than Structural Formula (100) and Structural Formula (150), are shown below.

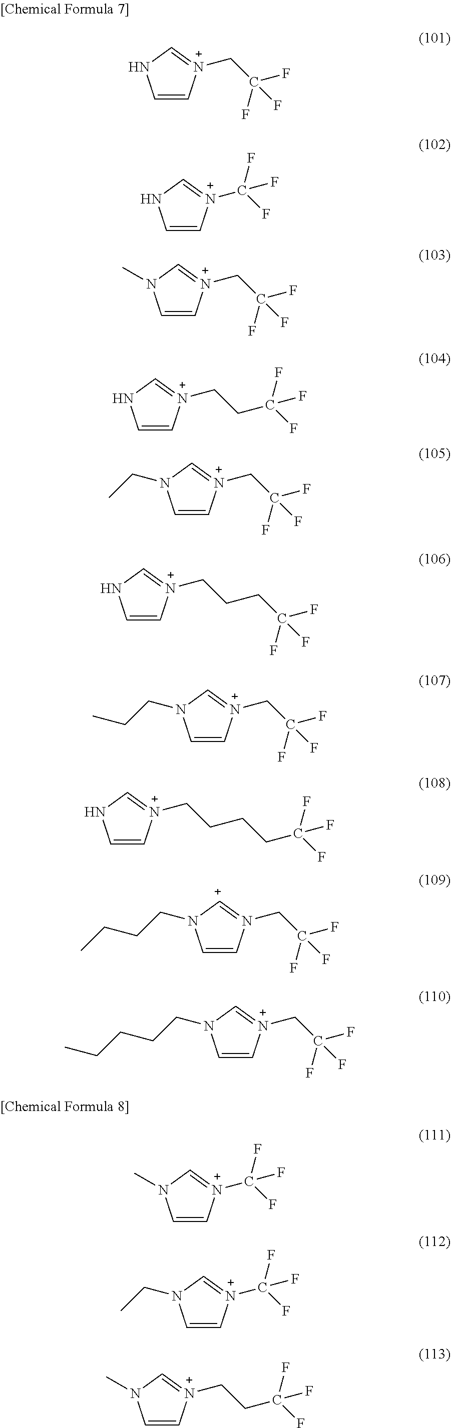

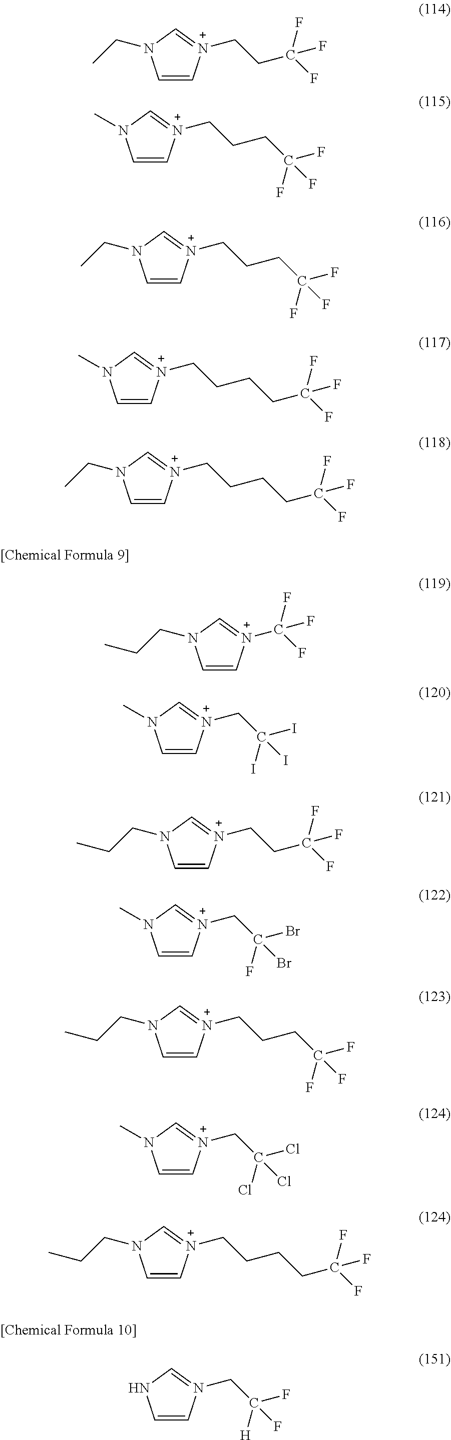

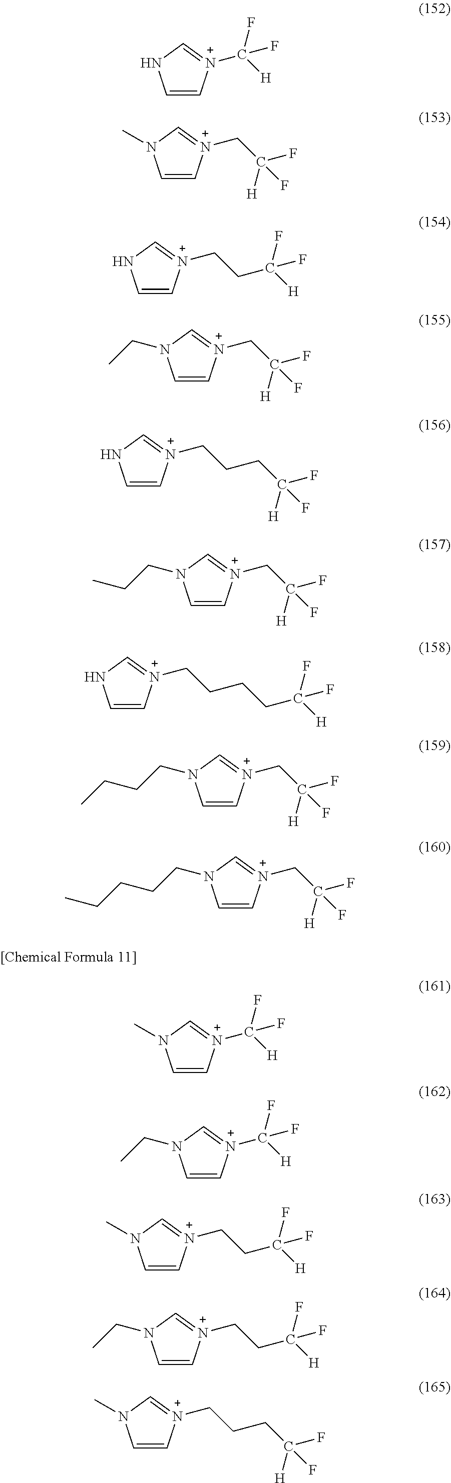

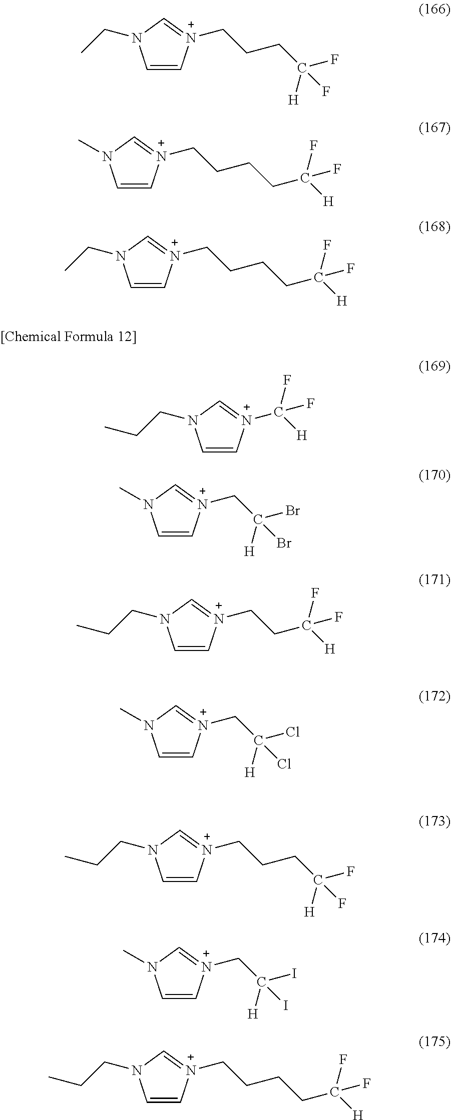

Note that the organic compounds represented by Structural Formulae (100) to (125) and Structural Formulae (150) to (175) above are examples of the organic compound represented by General Formula (G1) above, but the organic compound of one embodiment of the present invention is not limited thereto.

### <Synthesis Method of Ionic Liquid Example Including Cation Represented by General Formula (G1)>

A variety of reactions can be employed as a method for synthesizing the ionic liquid described in this embodiment. For example, the ionic liquid including the cation represented by General Formula (G1) can be synthesized by a synthesis method described below. Here, an example is described referring to synthesis schemes. Note that the method for synthesizing the ionic liquid described in this embodiment is not limited to the following synthesis methods.

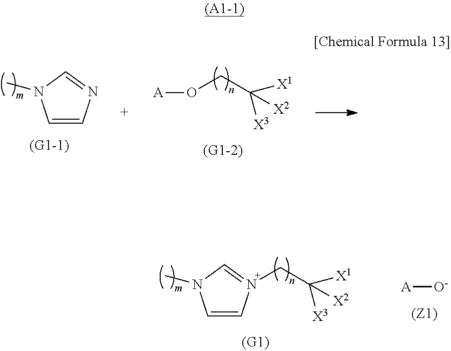

As shown in Scheme (A1-1) above, an imidazolium salt including the cation represented by General Formula (G1) and an anion (Z1) can be obtained from an imidazole derivative (G1-1) and a sulfonic acid compound (G1-2).

In Scheme (A1-1), X¹ to X³ each independently represent any one of fluorine, chlorine, bromine, and iodine. One of X¹ to X³ may be hydrogen. In addition, n and m each independently represent 0 to 5, and A represents a sulfonyl group.

Here, (G1-2) is not limited to the sulfonic acid compound but may be a halogen compound of alkoxyalkyl.

Scheme (A1-1) can be carried out with or without a solvent. Examples of a solvent that can be used in Scheme (A1-1) include nitriles such as acetonitrile, a halogen compound such as trichloroethane, alcohols such as ethanol and methanol, and ethers such as diethyl ether, tetrahydrofuran, and 1,4-dioxane. Note that the solvent that can be used is not limited thereto.

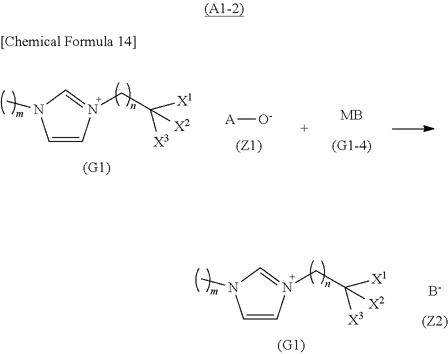

As shown in Scheme (A1-2) above, a variety of kinds of imidazolium salts can be obtained by exchanging ions between the imidazolium salt (G1, Z1) and a desired metallic salt (G1-4) including B.

In Scheme (A1-2), X¹ to X³ each independently represent any one of fluorine, chlorine, bromine, and iodine. One of X¹ to X³ may be hydrogen. In addition, n and m each independently represent 0 to 5, and A represents a sulfonyl group.

In Scheme (A1-2), B is any one of a monovalent amide anion, a monovalent methide anion, a fluorosulfonate anion (SO₃F⁻), a perfluoroalkylsulfonate anion, a tetrafluoroborate anion (BF₄⁻), a perfluoroalkylborate anion, a hexafluorophosphate anion (PF₆⁻), and a perfluoroalkylphosphate anion. Note that the anion that can be used is not limited thereto.

In Scheme (A1-2), M represents an alkali metal or the like. Examples of the alkali metal are, but not limited to, potassium, sodium, and lithium.

Scheme (A-2) can be carried out with or without a solvent. Examples of a solvent that can be used in Scheme (A-2) include water, alcohols such as ethanol and methanol, nitriles such as acetonitrile, and ethers such as diethyl ether, tetrahydrofuran, and 1,4-dioxane. Note that the solvent that can be used is not limited thereto.

In the above manner, the ionic liquid used for the secondary battery of one embodiment of the present invention can be formed. The ionic liquid of one embodiment of the present invention can be a nonaqueous solvent having flame resistance. The ionic liquid of one embodiment of the present invention can also be a nonaqueous solvent having high ionic conductivity. Thus, the secondary battery using the ionic liquid of one embodiment of the present invention can be a highly safe secondary battery with favorable charge and discharge rate characteristics.

Furthermore, the ionic liquid described above and an aprotic organic solvent may be mixed to be used as the electrolyte. As the aprotic organic solvent, for example, one of ethylene carbonate (EC), propylene carbonate (PC), butylene carbonate, chloroethylene carbonate, vinylene carbonate, γ-butyrolactone, γ-valerolactone, dimethyl carbonate (DMC), diethyl carbonate (DEC), ethyl methyl carbonate (EMC), methyl formate, methyl acetate, methyl butyrate, 1,3-dioxane, 1,4-dioxane, dimethoxyethane (DME), dimethyl sulfoxide, diethyl ether, methyl diglyme, acetonitrile, benzonitrile, tetrahydrofuran, sulfolane, sultone, and the like can be used, or two or more of them can be used in an appropriate combination at an appropriate ratio.

An additive agent such as succinonitrile, adiponitrile, vinylene carbonate (VC), propane sultone (PS), tert-butylbenzene (TBB), fluoroethylene carbonate (FEC), or lithium bis(oxalate)borate (LiBOB) may be added to the electrolyte. The concentration of the additive agent in the whole electrolyte is higher than or equal to 0.1 wt % and lower than or equal to 5 wt %. Note that the additive agent is reduced through a process such as aging in some cases.

As the lithium salt dissolved in the above-described solvent, one of lithium salts such as LiPF₆, LiClO₄, LiAsF₆, LiBF₄, LiAlCl₄, LiSCN, LiBr, LiI, Li₂SO₄, Li₂B₁₀Cl₁₀, Li₂B₁₂Cl₁₂, LiCF₃SO₃, LiC₄F₉SO₃, LiC(CF₃SO₂)₃, LiC(C₂F₅SO₂)₃, LiN (FSO₂)₂, LiN(CF₃SO₂)₂, LiN(C₄F₉SO₂) (CF₃SO₂), and LiN(C₂F₅SO₂)₂ can be used, or two or more of these lithium salts can be used in an appropriate combination at an appropriate ratio. The concentration of the electrolyte is preferably high, for example, higher than or equal to 0.8 mol/L and further preferably higher than or equal to 1.5 mol/L.

As the lithium salt, lithium bis(fluorosulfonyl)amide (abbreviation: LiFSA) or lithium bis(trifluoromethanesulfonyl)amide (abbreviation: LiTFSA) may be used. An electrolyte solution using LiFSA or LiTFSA as the electrolyte can inhibit elution of a metal in a positive electrode active material in a battery reaction of a power storage device. Thus, deterioration of the positive electrode active material and elution of the metal on a surface of a negative electrode are inhibited, so that the power storage device can achieve a small decrease in capacity and excellent cycle performance.

Note that the electrolyte using LiFSA or LiTFSA as the lithium salt reacts with and corrodes a positive electrode current collector in some cases. In order to prevent such corrosion, several weight percent of LiPF₆ is preferably added to the electrolyte. In that case, a passive film is formed on a surface of the positive electrode current collector and inhibits the reaction between the electrolyte and the positive electrode current collector. Note that the concentration of LiPF₆ is less than or equal to 10 wt %, preferably less than or equal to 5 wt %, and further preferably less than or equal to 3 wt % in order that the positive electrode active material layer is not dissolved.

Note that in the above lithium salts, an alkali metal (e.g., sodium and potassium), an alkaline-earth metal (e.g., calcium, strontium, barium, beryllium, and magnesium) may be used instead of lithium. That is, ions of these metals may be used as carrier ions.

The electrolyte is preferably a highly purified electrolyte that contains a small number of dust particles and elements other than the constituent elements of the electrolyte (hereinafter, also simply referred to as impurities). Specifically, the weight ratio of impurities to the electrolyte is preferably less than or equal to 1%, further preferably less than or equal to 0.1%, still further preferably less than or equal to 0.01%.

A gelled electrolyte in which a polymer is swelled with an electrolyte may also be used.

As the polymer, a polymer having a polyalkylene oxide structure, such as polyethylene oxide (PEO); polyvinylidene fluoride (PVdF); polyacrylonitrile; or a copolymer or the like containing any of them can be used. For example, PVdF-HFP, which is a copolymer of PVdF and hexafluoropropylene (HFP), can be used. The formed polymer may be porous.

An electrolyte may be gelled by adding a polymerization initiator and/or a cross-linking agent to the electrolyte. For example, the ionic liquid itself may be polymerized in such a manner that a polymerizable functional group is introduced into a cation or an anion of the ionic liquid and polymerization thereof is caused with the polymerization initiator. The polymerized ionic liquid may be gelled with a cross-linking agent in this manner.

In combination with the electrolyte, a solid electrolyte including an inorganic material such as a sulfide-based inorganic material and/or an oxide-based inorganic material, or a solid electrolyte including a high-molecular material such as a PEO (polyethylene oxide)-based material may be used. For example, the solid electrolyte may be formed over a surface of the active material layer. In the case where the solid electrolyte and the electrolyte are used in combination, a separator and/or a spacer does not need to be provided in some cases.

When a gelled high-molecular material is used for the electrolyte, safety against liquid leakage and the like is improved. Furthermore, the power storage device can be thinner and more lightweight. Typical examples of gelled high-molecular materials include a silicone gel, an acrylic gel, an acrylonitrile gel, a polyethylene oxide-based gel, a polypropylene oxide-based gel, and a gel of a fluorine-based polymer. Typical examples of high-molecular materials include a silicone gel, a polyacrylamide-based gel, a polyacrylonitrile-based gel, a polyethylene oxide-based gel, a polypropylene oxide-based gel, and a gel of a fluorine-based polymer.

For example, one embodiment of the present invention may include a positive electrode, a negative electrode, and an electrolyte; the electrolyte includes an ionic liquid containing the cation represented by General Formula (G1) above and the anion represented by Structural Formula (200); and the electrolyte may be gelled or solid.

This embodiment can be used in combination with the other embodiments.

### Embodiment 2

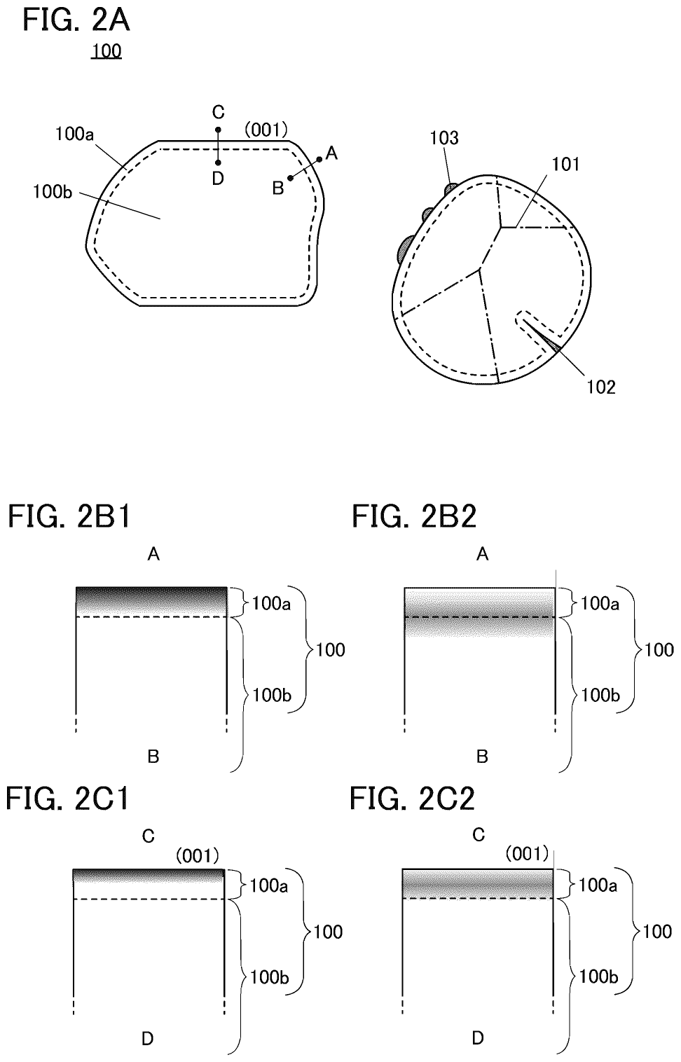

In this embodiment, a positive electrode active material that can be used for a secondary battery of one embodiment of the present invention is described with reference to FIG. 2A to FIG. 14.

### [Positive Electrode Active Material]

FIG. 2A is a cross-sectional view of a positive electrode active material 100 that can be used for a secondary battery of one embodiment of the present invention. FIG. 2B1 and FIG. 2B2 show enlarged views of a portion near the line A-B in FIG. 2A. FIG. 2C1 and FIG. 2C2 show enlarged views of a portion near the line C-D in FIG. 2A.

As illustrated in FIG. 2A to FIG. 2C2, the positive electrode active material 100 includes a surface portion 100a and an inner portion 100b. In each drawing, the dashed line denotes a boundary between the surface portion 100a and the inner portion 100b. In FIG. 2A, the dashed-dotted line denotes part of a crystal grain boundary 101.

In this specification and the like, a region that is approximately 10 nm in depth from the surface toward the inner portion of a positive electrode active material is referred to as the surface portion 100a. A plane generated by a split or a crack may also be referred to as a surface. The surface portion 100a may also be referred to as the vicinity of a surface, a region in the vicinity of a surface, or the like. A region in a deeper position than the surface portion 100a of the positive electrode active material is referred to as the inner portion 100b. The inner portion 100b may also be referred to as an inner region.

The surface portion 100a preferably has a higher concentration of an added element, which is described later, than the inner portion 100b. The added element preferably has a concentration gradient. In the case where a plurality of kinds of added elements are included, the added elements preferably exhibit concentration peaks at different depths from a surface.

For example, an added element X preferably has a concentration gradient as illustrated by gradation in FIG. 2B1, in which the concentration increases from the inner portion 100b toward the surface. Examples of the added element X that preferably has such a concentration gradient include magnesium, fluorine, titanium, silicon, phosphorus, boron, and calcium.

Another added element Y preferably has a concentration gradient as illustrated by gradation in FIG. 2B2 and exhibits a concentration peak at a deeper region than the added element X. The concentration peak may be located in the surface portion 100a or located deeper than the surface portion 100a. The concentration peak is preferably located in a region other than the outermost surface layer. For example, the concentration peak is preferably located in a region of 5 nm to 30 nm inclusive in depth from the surface. Examples of the added element Y that preferably has such a concentration gradient include aluminum and manganese.

It is preferable that the crystal structure continuously change from the inner portion 100b toward the surface owing to the above-described concentration gradient of the added element.

### <Contained Element>

The positive electrode active material 100 contains lithium, a transition metal M, oxygen, and an added element. The positive electrode active material 100 can be regarded as a composite oxide represented by LiMO₂ to which an added element is added. Note that the positive electrode active material of one embodiment of the present invention only needs to have a crystal structure of a lithium composite oxide represented by LiMO₂, and the composition is not strictly limited to Li:M:O=1:1:2. In some cases, a positive electrode active material to which an added element is added is referred to as a composite oxide.

As the transition metal M contained in the positive electrode active material 100, a metal that can form, together with lithium, a composite oxide having a layered rock-salt structure belonging to the space group R-3m is preferably used. For example, at least one of manganese, cobalt, and nickel can be used. That is, as the transition metal M contained in the positive electrode active material 100, cobalt may be used alone, nickel may be used alone, cobalt and manganese may be used, cobalt and nickel may be used, or cobalt, manganese, and nickel may be used. In other words, the positive electrode active material 100 can contain a composite oxide containing lithium and the transition metal M, such as lithium cobalt oxide, lithium nickel oxide, lithium cobalt oxide in which manganese is substituted for part of cobalt, lithium cobalt oxide in which nickel is substituted for part of cobalt, or lithium nickel-manganese-cobalt oxide.

Specifically, using cobalt at greater than or equal to 75 at %, preferably greater than or equal to 90 at %, further preferably greater than or equal to 95 at % as the transition metal M contained in the positive electrode active material 100 brings many advantages such as relatively easy synthesis, easy handling, and excellent cycle performance. Moreover, when nickel is contained as the transition metal M in addition to cobalt in the above range, a shift in a layered structure formed of octahedrons of cobalt and oxygen is sometimes inhibited. This is preferable because the crystal structure becomes more stable particularly in a charged state at a high temperature in some cases.

Note that manganese is not necessarily contained as the transition metal M. When the positive electrode active material 100 is substantially free from manganese, the above advantages such as relatively easy synthesis, easy handling, and excellent cycle performance are sometimes enhanced. The weight of manganese contained in the positive electrode active material 100 is preferably less than or equal to 600 ppm, further preferably less than or equal to 100 ppm, for example.

Using nickel at greater than or equal to 33 at %, preferably greater than or equal to 60 at %, further preferably greater than or equal to 80 at % as the transition metal M contained in the positive electrode active material 100 is preferable because in that case, the cost of the raw materials might be lower than that in the case of using a large amount of cobalt and charge and discharge capacity per weight might be increased.

Note that nickel is not necessarily contained as the transition metal M.

As the added element contained in the positive electrode active material 100, at least one of magnesium, fluorine, aluminum, titanium, zirconium, vanadium, iron, chromium, niobium, cobalt, arsenic, zinc, silicon, sulfur, phosphorus, and boron is preferably used. These added elements further stabilize the crystal structure of the positive electrode active material 100 in some cases as described later. The positive electrode active material 100 can contain lithium cobalt oxide to which magnesium and fluorine are added, lithium cobalt oxide to which magnesium, fluorine, and titanium are added, lithium nickel-cobalt oxide to which magnesium and fluorine are added, lithium cobalt-aluminum oxide to which magnesium and fluorine are added, lithium nickel-cobalt-aluminum oxide, lithium nickel-cobalt-aluminum oxide to which magnesium and fluorine are added, lithium nickel-manganese-cobalt oxide to which magnesium and fluorine are added, or the like. Note that in this specification and the like, the added element may be rephrased as a mixture, a constituent of a material, an impurity element, or the like.

Note that as the added element, magnesium, fluorine, aluminum, titanium, zirconium, vanadium, iron, chromium, niobium, cobalt, arsenic, zinc, silicon, sulfur, phosphorus, or boron is not necessarily contained.

In order to prevent breakage of a layered structure formed of octahedrons of the transition metal M and oxygen even when lithium is extracted from the positive electrode active material 100 of one embodiment of the present invention owing to charge, the surface portion 100a having a high additive-element concentration, i.e., the outer portion of the particle, is reinforced.

Note that the added elements do not necessarily have similar concentration gradients throughout the surface portion 100a of the positive electrode active material 100. FIG. 2C1 shows an example of distribution of the added element Xin a portion near the line C-D in FIG. 2A. FIG. 2C2 shows an example of distribution of the added element Yin a portion near the line C-D.

Here, the portion near the line C-D has a layered rock-salt crystal structure belonging to R-3m and the surface of the portion has a (001) orientation. The distribution of the added element at the surface having a (001) orientation may be different from that at other surfaces. For example, at least one of the added element X and the added element Y may be distributed shallower from the surface having a (001) orientation and the surface portion 100a thereof than from a surface having an orientation other than a (001) orientation. Alternatively, the surface having a (001) orientation and the surface portion 100a thereof may have a lower concentration of at least one of the added element X and the added element Y than a surface having an orientation other than a (001) orientation. Further alternatively, at the surface having a (001) orientation and the surface portion 100a thereof, the concentration of at least one of the added element X and the added element Y may be below the lower detection limit.

In a layered rock-salt crystal structure belonging to R-3m, cations are arranged parallel to a (001) plane. In other words, an MO₂ layer formed of octahedrons of the transition metal M and oxygen and a lithium layer are alternately stacked parallel to a (001) plane. Accordingly, a diffusion path of lithium ions also exists parallel to a (001) plane.

The MO₂ layer formed of octahedrons of the transition metal M and oxygen is relatively stable and thus, the surface of the positive electrode active material 100 is more stable when having a (001) orientation. A diffusion path of lithium ions is not exposed at a (001) plane.

By contrast, a diffusion path of lithium ions is exposed at a surface having an orientation other than a (001) orientation. Thus, the surface having an orientation other than a (001) orientation and the surface portion 100a thereof easily lose stability because they are regions where extraction of lithium ions starts as well as important regions for maintaining a diffusion path of lithium ions. It is thus extremely important to reinforce the surface having an orientation other than a (001) orientation and the surface portion 100a thereof so that the crystal structure of the whole positive electrode active material 100 is maintained.

Accordingly, in the positive electrode active material 100 of another embodiment of the present invention, it is important to distribute the added element at the surface having an orientation other than a (001) orientation and the surface portion 100a thereof as shown in FIG. 2B1 or 2B2. By contrast, in the surface having a (001) orientation and the surface portion 100a thereof, the added element may have a peak at a shallow position or a low concentration as described above or the added element may be absent.

In the formation method as described later, in which high-purity LiMO₂ is formed, the added element is mixed afterwards, and heating is performed, the added element moves to the surface portion mainly via a diffusion path of lithium ions and thus, distribution of the added element at the surface having an orientation other than a (001) orientation and the surface portion 100a thereof can easily have a high concentration.

By the formation method in which high-purity LiMO₂ is formed, the added element is then mixed, and heating is performed, the added element can have a preferable distribution in a surface having an orientation other than a (001) orientation and the surface portion 100a thereof as compared to in a surface having a (001) orientation. Moreover, in the formation method involving the initial heating, lithium atoms in the surface portion are expected to be extracted from LiMO₂ owing to the initial heating and thus, the added element such as Mg atoms can be probably distributed easily in the surface portion at a high concentration.

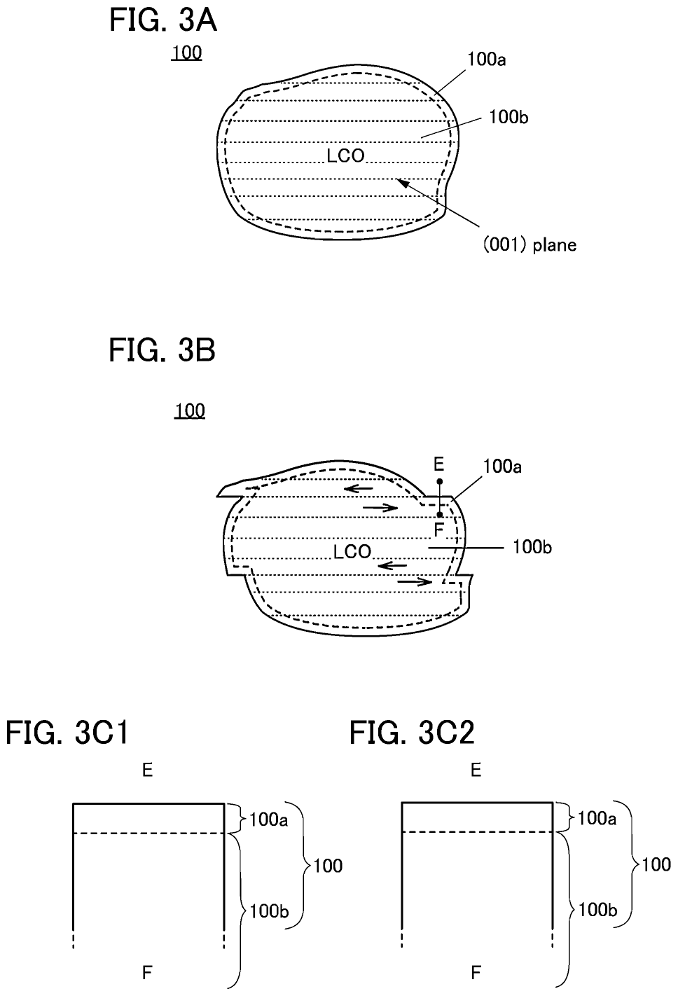

The positive electrode active material 100 preferably has a smooth surface with little unevenness; however, it is not necessary that the whole surface of the positive electrode active material 100 be in such a state. In a composite oxide with a layered rock-salt crystal structure belonging to R-3m, slipping easily occurs at a plane parallel to a (001) plane, e.g., a plane where lithium atoms are arranged. In the case where a (001) plane is horizontal as shown in FIG. 3A, steps such as a pressing step sometimes cause slipping in a horizontal direction as denoted by arrows in FIG. 3B, resulting in deformation.

In this case, at a surface newly formed as a result of slipping and the surface portion 100a thereof, the added element does not exist or the concentration of the added element is below the lower detection limit in some cases. The line E-F in FIG. 3B denotes examples of the surface newly formed as a result of slipping and its surface portion 100a. FIG. 3C1 and FIG. 3C2 show enlarged views of the vicinity of the line E-F. In FIG. 3C1 and FIG. 3C2, additive in FIG. 2B1 to FIG. 2C2, there exists neither gradation of the added element X nor that of the added element Y.

However, because slipping easily occurs parallel to a (001) plane, the newly formed surface and the surface portion 100a thereof have a (001) orientation. Since a diffusion path of lithium ions is not exposed at a (001) plane and the surface having a (001) plane is relatively stable, substantially no problem is caused even when the added element does not exist or the concentration of the added element is below the lower detection limit in the surface having a (001) plane.

Note that as described above, in a composite oxide whose composition is LiMO₂ and which has a layered rock-salt crystal structure belonging to R-3m, cations are arranged parallel to a (001) plane. In a HAADF-STEM image or the like, the luminance of the transition metal M, which has the largest atom number in LiMO₂, is the highest. Thus, in a HAADF-STEM image or the like, arrangement of atoms with a high luminance may be regarded as arrangement of atoms of the transition metal M. Repetition of such arrangement with a high luminance may be referred to as crystal fringes or lattice fringes. Such crystal fringes or lattice fringes may be deemed to be parallel to a (001) plane in the case of a layered rock-salt crystal structure belonging to R-3m.

The positive electrode active material 100 has a depression, a crack, a concave, a V-shaped cross section, or the like in some cases. These are examples of defects, and when charge and discharge are repeated, elution of the transition metal M, breakage of a crystal structure, cracking of the positive electrode active material 100, extraction of oxygen, or the like might be derived from these defects. However, when there is a filling portion 102 that fills such defects, elution of the transition metal M or the like can be inhibited. Thus, the positive electrode active material 100 can have high reliability and excellent cycle performance.

The positive electrode active material 100 may include a projection 103, which is a region where the added element is unevenly distributed.

As described above, an excessive amount of the added element in the positive electrode active material 100 might adversely affect insertion and extraction of lithium. The use of such a positive electrode active material 100 for a secondary battery might cause an internal resistance increase, a charge and discharge capacity decrease, and the like. Meanwhile, when the amount of the added element is insufficient, the added element is not distributed throughout the surface portion 100a, which might diminish the effect of inhibiting degradation of a crystal structure. The added element (also referred to as the impurity element) is thus required to be contained in the positive electrode active material 100 at an appropriate concentration; however, the adjustment of the concentration is not easy.

For this reason, when the positive electrode active material 100 includes the region where the impurity element is unevenly distributed, part of the excess impurity is removed from the inner portion 100b, so that the impurity concentration can be appropriate in the inner portion 100b. This can inhibit an internal resistance increase, a charge and discharge capacity decrease, and the like when a secondary battery is fabricated. A feature of inhibiting an internal resistance increase in a secondary battery is extremely preferable especially in charge and discharge at a high rate such as charge and discharge at 2 C or more.

In the positive electrode active material 100 including the region where the impurity element is unevenly distributed, addition of excess impurities to some extent in the formation process is acceptable. This is preferable because the margin of production can be increased.

In this specification and the like, uneven distribution refers to a state where the concentration of a certain element in a certain region is different from that in other regions, and may be rephrased as segregation, precipitation, unevenness, deviation, a mixture of a high-concentration portion and a low-concentration portion, or the like.

Magnesium, which is one of the added element X, is divalent and is more stable in lithium sites than in transition metal sites in a layered rock-salt crystal structure; thus, magnesium is likely to enter the lithium sites. An appropriate concentration of magnesium in the lithium sites of the surface portion 100a facilitates maintenance of the layered rock-salt crystal structure. Magnesium can inhibit extraction of oxygen around magnesium at the time of high voltage charge. Magnesium is also expected to increase the density of the positive electrode active material. An appropriate concentration of magnesium does not have an adverse effect on insertion or extraction of lithium in charge and discharge, and is thus preferable. However, excess magnesium might adversely affect insertion and extraction of lithium. Thus, as will be described later, the concentration of the transition metal M is preferably higher than that of magnesium in the surface portion 100a, for example.

Aluminum, which is an example of the added element Y, is trivalent and can exist at a transition metal site in a layered rock-salt crystal structure. Aluminum can inhibit elution of surrounding cobalt. The bonding strength of aluminum with oxygen is high, thereby inhibiting extraction of oxygen around aluminum. Hence, aluminum contained as the added element enables the positive electrode active material 100 to have the crystal structure that is unlikely to be broken by repeated charge and discharge.

When fluorine, which is a monovalent anion, is substituted for part of oxygen in the surface portion 100a, the lithium extraction energy is lowered. This is because the change in valence of cobalt ions associated with lithium extraction is trivalent to tetravalent in the case of not containing fluorine and divalent to trivalent in the case of containing fluorine, and the oxidation-reduction potential differs therebetween. It can thus be said that when fluorine is substituted for part of oxygen in the surface portion 100a of the positive electrode active material 100, lithium ions near fluorine are likely to be extracted and inserted smoothly. Thus, using such a positive electrode active material 100 in a secondary battery is preferable because the charge and discharge characteristics, rate performance, and the like are improved.

Titanium oxide is known to have superhydrophilicity. Accordingly, the positive electrode active material 100 including titanium oxide at the surface portion 100a presumably has good wettability with respect to a high-polarity electrolyte like the ionic liquid of one embodiment of the present invention. When used for a secondary battery, the positive electrode active material 100 and the electrolyte can have favorable contact at the interface therebetween and presumably inhibit an internal resistance increase.

The voltage of a positive electrode generally increases with increasing charge voltage of a secondary battery. The positive electrode active material of one embodiment of the present invention has a stable crystal structure even at a high voltage. The stable crystal structure of the positive electrode active material in a charged state can suppress a charge and discharge capacity decrease due to repeated charge and discharge.

A short circuit of a secondary battery might cause not only malfunction in charging operation and discharging operation of the secondary battery but also heat generation and firing. In order to obtain a safe secondary battery, a short-circuit current is preferably inhibited even at a high charge voltage. In the positive electrode active material 100 of one embodiment of the present invention, a short-circuit current is inhibited even at a high charge voltage. Thus, a secondary battery having high charge and discharge capacity and a high level of safety can be obtained.

The concentration gradient of the added element can be evaluated using energy dispersive X-ray spectroscopy (EDX), EPMA (electron probe microanalysis), or the like. In the EDX measurement, the measurement in which a region is measured while scanning the region and evaluated two-dimensionally is referred to as EDX surface analysis. The measurement by line scan, which is performed to evaluate the atomic concentration distribution in a positive electrode active material particle, is referred to as linear analysis. Furthermore, extracting data of a linear region from EDX surface analysis is referred to as linear analysis in some cases. The measurement of a region without scanning is referred to as point analysis.

By EDX surface analysis (e.g., element mapping), the concentrations of the added element in the surface portion 100a, the inner portion 100b, the vicinity of a crystal grain boundary, and the like of the positive electrode active material 100 can be quantitatively analyzed. By EDX linear analysis, the concentration distribution and the highest concentration of the added element can be analyzed.

When the positive electrode active material 100 containing magnesium as the added element is subjected to the EDX linear analysis, a peak of the magnesium concentration in the surface portion 100a is preferably exhibited by a region that is 3 nm in depth, further preferably 1 nm in depth, still further preferably 0.5 nm in depth from the surface toward the center of the positive electrode active material 100.

When the positive electrode active material 100 contains magnesium and fluorine as the added elements, the distribution of fluorine preferably overlaps with the distribution of magnesium. Thus, in the EDX linear analysis, a peak of the fluorine concentration in the surface portion 100a is preferably exhibited by a region that is 3 nm in depth, further preferably 1 nm in depth, still further preferably 0.5 nm in depth from the surface toward the center of the positive electrode active material 100.

Note that the concentration distribution may differ between the added elements. For example, in the case where the positive electrode active material 100 contains aluminum as the added element, the distribution of aluminum is preferably slightly different from that of magnesium and that of fluorine as described above. For example, in the EDX linear analysis, the peak of the magnesium concentration is preferably closer to the surface than the peak of the aluminum concentration is in the surface portion 100a. For example, the peak of the aluminum concentration is preferably exhibited by a region that is greater than or equal to 0.5 nm and less than or equal to 50 nm in depth, further preferably greater than or equal to 5 nm and less than or equal to 30 nm in depth from the surface toward the center of the positive electrode active material 100. Alternatively, the peak of the aluminum concentration is preferably exhibited by a region that is greater than or equal to 0.5 nm and less than or equal to 30 nm in depth from the surface toward the center of the positive electrode active material 100. Further alternatively, the peak of the aluminum concentration is preferably exhibited by a region that is greater than or equal to 5 nm and less than or equal to 50 nm in depth from the surface toward the center of the positive electrode active material 100.

When the positive electrode active material 100 is subjected to linear analysis or surface analysis, the atomic ratio of an impurity element I to the transition metal M (I/M) in the surface portion 100a is preferably greater than or equal to 0.05 and less than or equal to 1.00. When the impurity element is titanium, the atomic ratio of titanium to the transition metal M (Ti/M) is preferably greater than or equal to 0.05 and less than or equal to 0.4, further preferably greater than or equal to 0.1 and less than or equal to 0.3. When the impurity element is magnesium, the atomic ratio of magnesium to the transition metal M (Mg/M) is preferably greater than or equal to 0.4 and less than or equal to 1.5, further preferably greater than or equal to 0.45 and less than or equal to 1.00. When the impurity element is fluorine, the atomic ratio of fluorine to the transition metal M (F/M) is preferably greater than or equal to 0.05 and less than or equal to 1.5, further preferably greater than or equal to 0.3 and less than or equal to 1.00.

According to results of the EDX linear analysis, where a surface of the positive electrode active material 100 is can be estimated as follows. A point where the detected amount of an element which uniformly exists in the inner portion 100b of the positive electrode active material 100, e.g., oxygen or the transition metal M such as cobalt, is ½ of the detected amount thereof in the inner portion 100b is assumed as the surface.

Since the positive electrode active material 100 is a composite oxide, the detected amount of oxygen is preferably used to estimate where the surface is. Specifically, an average value Oₐᵥₑ of the oxygen concentration of a region of the inner portion 100b where the detected amount of oxygen is stable is calculated first. At this time, in the case where oxygen O_{background} which is probably led from chemical adsorption or the background is detected in a region that is obviously outside the surface, O_{background} is subtracted from the measurement value to obtain the average value Oₐᵥₑ of the oxygen concentration. The measurement point where the measurement value which is closest to ½ of the average value Oₐᵥₑ, or ½Oₐᵥₑ, is obtained can be estimated to be the surface of the positive electrode active material.

Where the surface is can also be estimated with the use of the transition metal M contained in the positive electrode active material 100. For example, in the case where 95% or more of the transition metals M is cobalt, the detected amount of cobalt can be used to estimate where the surface is as in the above description. Alternatively, the sum of the detected amounts of the transition metals M can be used for the estimation in a similar manner. The detected amount of the transition metal M is unlikely to be affected by chemical adsorption and is thus suitable for the estimation of where the surface is.

When the positive electrode active material 100 is subjected to linear analysis or surface analysis, the atomic ratio of the added element I to the transition metal M (I/M) in the vicinity of the crystal grain boundary is preferably greater than or equal to 0.020 and less than or equal to 0.50, further preferably greater than or equal to 0.025 and less than or equal to 0.30, still further preferably greater than or equal to 0.030 and less than or equal to 0.20. Alternatively, it is preferably greater than or equal to 0.020 and less than or equal to 0.30. Alternatively, it is preferably greater than or equal to 0.020 and less than or equal to 0.20. Alternatively, it is preferably greater than or equal to 0.025 and less than or equal to 0.50. Alternatively, it is preferably greater than or equal to 0.025 and less than or equal to 0.20. Alternatively, it is preferably greater than or equal to 0.030 and less than or equal to 0.50. Alternatively, it is preferably greater than or equal to 0.030 and less than or equal to 0.30.

For example, when the added element is magnesium and the transition metal M is cobalt, the atomic ratio of magnesium to cobalt (Mg/Co) is preferably greater than or equal to 0.020 and less than or equal to 0.50, further preferably greater than or equal to 0.025 and less than or equal to 0.30, still further preferably greater than or equal to 0.030 and less than or equal to 0.20. Alternatively, it is preferably greater than or equal to 0.020 and less than or equal to 0.30. Alternatively, it is preferably greater than or equal to 0.020 and less than or equal to 0.20. Alternatively, it is preferably greater than or equal to 0.025 and less than or equal to 0.50. Alternatively, it is preferably greater than or equal to 0.025 and less than or equal to 0.20. Alternatively, it is preferably greater than or equal to 0.030 and less than or equal to 0.50. Alternatively, it is preferably greater than or equal to 0.030 and less than or equal to 0.30.

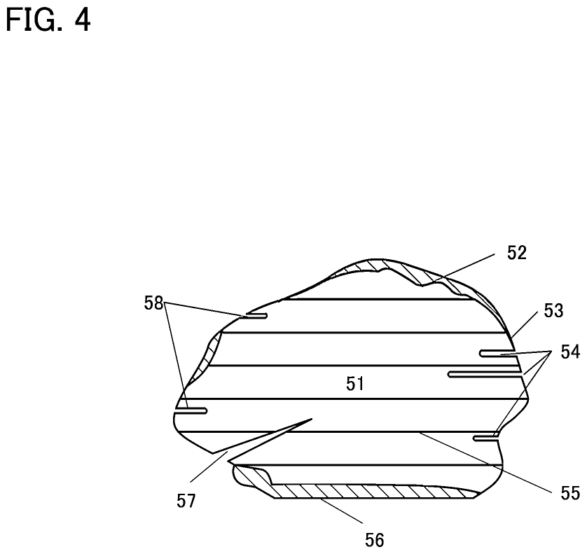

Note that when the positive electrode active material 100 undergoes charge and discharge under high-voltage conditions at 4.5 V or more, or at a high temperature (45° C. or higher), a progressive defect (also referred to as a pit) might be generated in the positive electrode active material. In addition, a defect such as a crevice (also referred to as a crack) might be generated by expansion and contraction of the positive electrode active material due to charge and discharge. FIG. 4 shows a schematic cross-sectional view of a positive electrode active material particle 51. Although pits of the positive electrode active material particle 51 are illustrated as holes denoted by reference numerals 54 and 58, their opening shape is not circular but a wide groove-like shape. A source of a pit can be a point defect. Presumably, the crystal structure of LiMO₂ in the vicinity of a portion where a pit is formed is broken and differs from a layered rock-salt crystal structure. The breakage of the crystal structure might inhibit diffusion and release of lithium ions that are carrier ions; thus, a pit is probably a cause of degradation of cycle performance. A crack of the positive electrode active material particle 51 is denoted by a reference numeral 57. A reference numeral 55 denotes a crystal plane, a reference numeral 52 denotes a depression, and reference numerals 53 and 56 denote regions where the added element exists.

Typical positive electrode active materials of lithium-ion secondary batteries are LCO and NCM, which can also be regarded as an alloy containing a plurality of metal elements (cobalt, nickel, and the like). At least one of a plurality of positive electrode active material particles has a defect and the defect might change before and after charge and discharge. When used in a secondary battery, a positive electrode active material particle might undergo a phenomenon such as chemical or electrochemical erosion or degradation due to environmental substances (e.g., electrolyte) surrounding the positive electrode active material particle. This degradation does not occur uniformly in the surface of the particle but occurs locally in a concentrated manner, and a defect is formed deeply from the surface toward the inner portion, for example, by repeated charge and discharge of the secondary battery.

Progress of a defect in a positive electrode active material to form a hole can be referred to as pitting corrosion, and the hole generated by this phenomenon is also referred to as a pit in this specification.

In this specification, a crack and a pit are different from each other. Immediately after formation of a positive electrode active material particle, a crack can exist but a pit does not exist. A pit can also be regarded as a hole formed by extraction of some layers of cobalt and oxygen due to charge and discharge under high-voltage conditions at 4.5 V or more, or at a high temperature (45° C. or higher), i.e., a portion from which cobalt has been eluted. A crack refers to a surface newly generated by application of physical pressure or a crevice generated owing to the crystal grain boundary. A crack might be caused by expansion and contraction of the particle due to charge and discharge. A pit might be generated from a crack and a cavity in the particle.

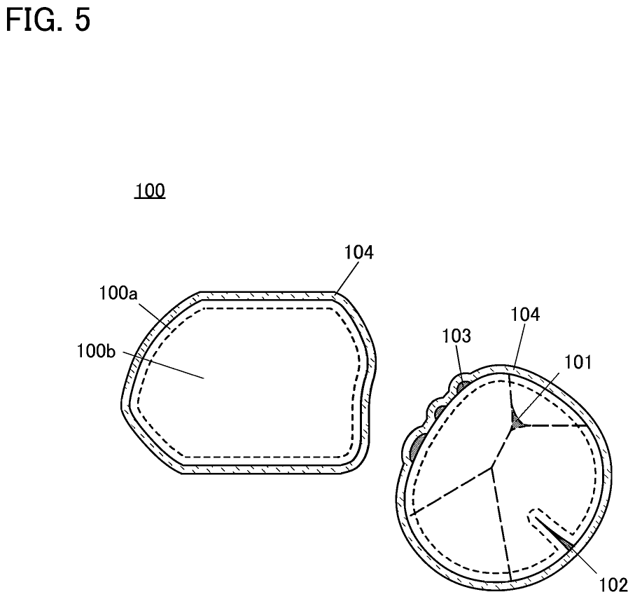

The positive electrode active material 100 may include a coating film in at least part of its surface. FIG. 5 shows an example of the positive electrode active material 100 including a coating film 104.

The coating film 104 is preferably formed by deposition of a decomposition product of an electrolyte due to charge and discharge, for example. A coating film originating from an electrolyte, which is formed on the surface of the positive electrode active material 100, is expected to improve charge and discharge cycle performance particularly when high voltage charge is repeated. This is because an increase in impedance of the surface of the positive electrode active material is inhibited or elution of the transition metal M is inhibited, for example. The coating film 104 preferably contains carbon, oxygen, and fluorine, for example. The coating film can have high quality easily when the electrolyte includes LiBOB and/or SUN (suberonitrile), for example. Accordingly, the coating film 104 possibly has high quality when containing at least one of boron, nitrogen, sulfur, and fluorine. The coating film 104 does not necessarily cover the positive electrode active material 100 entirely.

### <Crystal Structure>

A material with a layered rock-salt crystal structure, such as lithium cobalt oxide (LiCoO₂), is known to have a high discharge capacity and excel as a positive electrode active material of a secondary battery. Examples of a material with a layered rock-salt crystal structure include a composite oxide represented by LiMO₂.

It is known that the Jahn-Teller effect in a transition metal compound varies in degree according to the number of electrons in the d orbital of the transition metal.

In a compound containing nickel, distortion is likely to be caused because of the Jahn-Teller effect in some cases. Accordingly, when high voltage charge and discharge are performed on LiNiO₂, the crystal structure might be broken because of the distortion. The influence of the Jahn-Teller effect is suggested to be small in LiCoO₂; hence, LiCoO₂ is preferable because the tolerance at the time of high voltage charge is higher in some cases.

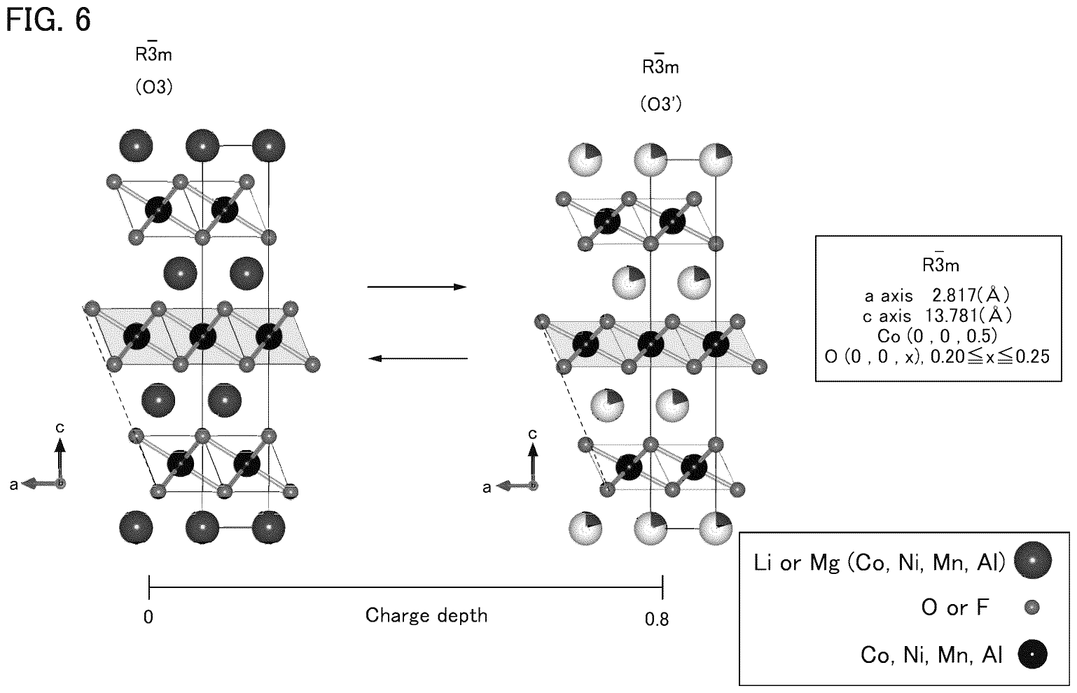

Crystal structures of positive electrode active materials are described with reference to FIG. 6 to FIG. 9. In FIG. 6 to FIG. 9, the case where cobalt is used as the transition metal M contained in the positive electrode active material is described.

### <Conventional Positive Electrode Active Material>

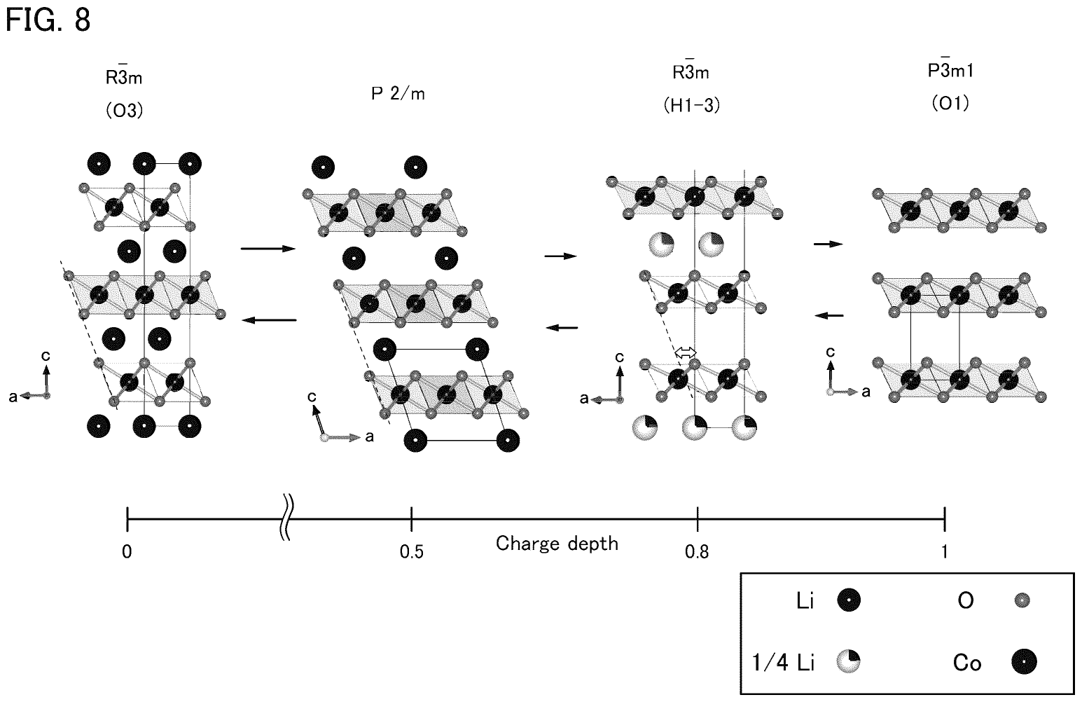

A positive electrode active material shown in FIG. 8 is lithium cobalt oxide (LiCoO₂) to which fluorine and magnesium are not added in a formation method described later. As described in Non-Patent Document 1, Non-Patent Document 2, and the like, the crystal structure of the lithium cobalt oxide shown in FIG. 8 changes with the charge depth.

As shown in FIG. 8, in lithium cobalt oxide with a charge depth of 0 (discharged state), there is a region having a crystal structure belonging to the space group R-3m, lithium occupies octahedral sites, and a unit cell includes three CoO₂ layers. Thus, this crystal structure is referred to as an O3 type crystal structure in some cases. Note that here, the CoO₂ layer has a structure in which an octahedral structure with cobalt coordinated to six oxygen atoms continues on a plane in an edge-shared state.

Lithium cobalt oxide with a charge depth of 1 has the crystal structure belonging to the space group P-3 ml and includes one CoO₂ layer in a unit cell. Hence, this crystal structure is referred to as an O1 type crystal structure in some cases.

Lithium cobalt oxide with a charge depth of approximately 0.8 has the crystal structure belonging to the space group R-3m. This structure can also be regarded as a structure in which CoO₂ structures such as P-3 ml (O1) and LiCoO₂ structures such as R-3m (O3) are alternately stacked. Thus, this crystal structure is referred to as an H1-3 type crystal structure in some cases. Note that the number of cobalt atoms per unit cell in the actual H1-3 type crystal structure is twice that in other structures. However, in this specification, FIG. 8, and other drawings, the c-axis of the H1-3 type crystal structure is half that of the unit cell for easy comparison with the other crystal structures.

For the H1-3 type crystal structure, as disclosed in Non-Patent Document 3, the coordinates of cobalt and oxygen in the unit cell can be expressed as follows, for example: Co (0, 0, 0.42150±0.00016), O1 (0, 0, 0.27671±0.00045), and 02 (0, 0, 0.11535±0.00045). O1 and 02 are each an oxygen atom. In this manner, the H1-3 type crystal structure is represented by a unit cell including one cobalt atom and two oxygen atoms. Meanwhile, the O3′ type crystal structure of one embodiment of the present invention is preferably represented by a unit cell including one cobalt atom and one oxygen atom, as described later. This means that the symmetry of cobalt and oxygen differs between the O3′ structure and the H1-3 type structure, and the amount of change from the O3 structure is smaller in the O3′ structure than in the H1-3 type structure. A preferred unit cell for representing a crystal structure in a positive electrode active material is selected such that the value of GOF (goodness of fit) is smaller in Rietveld analysis of XRD patterns, for example.

When charge at a high voltage of 4.6 V or more with reference to the redox potential of a lithium metal or charge with a large charge depth of 0.8 or more and discharge are repeated, the crystal structure of lithium cobalt oxide changes (i.e., an unbalanced phase change occurs) repeatedly between the H1-3 type crystal structure and the R-3m (O3) structure in a discharged state.

However, there is a large shift in the CoO₂ layers between these two crystal structures. As denoted by the dotted lines and the arrow in FIG. 8, the CoO₂ layer in the H1-3 type crystal structure largely shifts from that in the R-3m (O3) structure. Such a dynamic structural change can adversely affect the stability of the crystal structure.

A difference in volume is also large. The O3 type crystal structure in a discharged state and the H1-3 type crystal structure that contain the same number of cobalt atoms have a difference in volume of 3.0% or more.

In addition, a structure in which CoO₂ layers are arranged continuously, such as P-3m1 (O1), included in the H1-3 type crystal structure is highly likely to be unstable.

Accordingly, the repeated high voltage charge and discharge gradually break the crystal structure of lithium cobalt oxide. The broken crystal structure triggers deterioration of the cycle performance. The broken crystal structure reduces sites where lithium can exist stably and makes it difficult to insert and extract lithium.

### <Positive Electrode Active Material Used in Secondary Battery of One Embodiment of the Present Invention>

### <<Crystal Structure>>

In the positive electrode active material 100 that can be used in a secondary battery of one embodiment of the present invention, the shift in CoO₂ layers can be small in repeated high voltage charge and discharge. Furthermore, the change in the volume can be small. Accordingly, the positive electrode active material that can be used in a secondary battery of one embodiment of the present invention can achieve excellent cycle performance. In addition, the positive electrode active material that can be used in a secondary battery of one embodiment of the present invention can have a stable crystal structure in a high voltage charged state. Thus, the positive electrode active material that can be used in a secondary battery of one embodiment of the present invention inhibits a short circuit in some cases while the high voltage charged state is maintained. This is preferable because the safety is further improved.

The positive electrode active material that can be used in a secondary battery of one embodiment of the present invention has a small crystal-structure change and a small volume difference per the same number of atoms of the transition metal M between a sufficiently discharged state and a high voltage charged state.

FIG. 6 shows a crystal structure of the positive electrode active material 100 before and after charge and discharge. The positive electrode active material 100 is a composite oxide containing lithium, cobalt as the transition metal M, and oxygen. In addition to the above-described elements, magnesium is preferably contained as the additive. Furthermore, fluorine is preferably contained as the additive.

The crystal structure with a charge depth of 0 (discharged state) in FIG. 6 is R-3m (O3), which is the same as in FIG. 8. Meanwhile, the positive electrode active material 100 with a charge depth in a sufficiently charged state includes a crystal whose structure is different from the H1-3 type crystal structure. This structure belongs to the space group R-3m and is a structure in which an ion of cobalt, magnesium, or the like occupies a site coordinated to six oxygen atoms. Furthermore, the symmetry of CoO₂ layers of this structure is the same as that in the O3 type structure. This structure is thus referred to as the O3′ type crystal structure in this specification and the like. In both the O3 type crystal structure and the O3′ type crystal structure, a slight amount of magnesium preferably exists between the CoO₂ layers, i.e., in lithium sites. In addition, a slight amount of fluorine preferably exists at random in oxygen sites.

Note that in the O3′ type crystal structure, a light element such as lithium sometimes occupies a site coordinated to four oxygen atoms.

Although a chance of the existence of lithium is the same in all lithium sites in FIG. 6, the positive electrode active material 100 of one embodiment of the present invention is not limited thereto. Lithium may exist unevenly in only some of the lithium sites. For example, lithium may exist in some lithium sites that are aligned, as in Li_{0.5}CoO₂ belonging to the space group P2/m. Distribution of lithium can be analyzed by neutron diffraction, for example.

The O3′ type crystal structure can be regarded as a crystal structure that contains Li between layers randomly but is similar to a CdCl₂ type crystal structure. The crystal structure similar to the CdCl₂ type crystal structure is close to a crystal structure of lithium nickel oxide (Li_{0.06}NiO₂) that is charged until the charge depth reaches 0.94; however, pure lithium cobalt oxide or a layered rock-salt positive electrode active material containing a large amount of cobalt is known not to have such a crystal structure generally.

In the positive electrode active material 100 that can be used in a secondary battery of one embodiment of the present invention, a change in the crystal structure caused when a large amount of lithium is extracted by high voltage charge is smaller than that in a conventional positive electrode active material. As denoted by the dotted lines in FIG. 6, for example, the CoO₂ layers hardly shift between the crystal structures.

Specifically, the crystal structure of the positive electrode active material 100 that can be used in a secondary battery of one embodiment of the present invention is highly stable even when a charge voltage is high. For example, at a charge voltage that makes a conventional positive electrode active material have the H1-3 type crystal structure, for example, at a voltage of approximately 4.6 V with reference to the potential of a lithium metal, the crystal structure belonging to R-3m (O3) can be maintained. Moreover, in a higher charge voltage range, for example, at voltages of greater than or equal to 4.65 V and less than or equal to 4.7 V with reference to the potential of a lithium metal, the O3′ type crystal structure can be obtained. At a much higher charge voltage, the H1-3 type crystal structure is eventually observed in some cases. In addition, the positive electrode active material 100 of one embodiment of the present invention might have the O3′ type crystal structure even at a lower charge voltage (e.g., a charge voltage of greater than or equal to 4.5 V and less than 4.6 V with reference to the potential of a lithium metal).

Thus, in the positive electrode active material 100 that can be used in a secondary battery of one embodiment of the present invention, the crystal structure is unlikely to be broken even when high voltage charge and discharge are repeated.

The space group of a crystal structure is identified by XRD, electron diffraction, neutron diffraction, or the like. Thus, in this specification and the like, belonging to a space group or being a space group can be rephrased as being identified as a space group.

Note that in the case where graphite is used as a negative electrode active material in a secondary battery, for example, the voltage of the secondary battery is lower than the above-mentioned voltages by the potential of graphite. The potential of graphite is approximately 0.05 V to 0.2 V with reference to the potential of a lithium metal. Thus, even in a secondary battery which includes graphite as a negative electrode active material and which has a voltage of greater than or equal to 4.3 V and less than or equal to 4.5 V, for example, the positive electrode active material 100 of one embodiment of the present invention can maintain the R-3m (O3) crystal structure and moreover, can have the O3′ type crystal structure at higher charge voltages, e.g., a voltage of the secondary battery of greater than 4.5 V and less than or equal to 4.6 V. In addition, the positive electrode active material 100 of one embodiment of the present invention can have the O3′ type crystal structure at lower charge voltages, e.g., at a voltage of the secondary battery of greater than or equal to 4.2 V and less than 4.3 V, in some cases.

Note that in the unit cell of the O3′ type crystal structure, the coordinates of cobalt and oxygen can be represented by Co (0, 0, 0.5) and O (0, 0, x) within the range of 0.20×0.25.

A slight amount of the added element such as magnesium randomly existing between the CoO₂ layers, i.e., in lithium sites, can suppress a shift in the CoO₂ layers at the time of high voltage charge. Thus, magnesium between the CoO₂ layers makes it easier to obtain the O3′ type crystal structure. Therefore, magnesium is preferably distributed throughout a particle of the positive electrode active material 100 of one embodiment of the present invention. To distribute magnesium throughout the particle, heat treatment is preferably performed in the formation process of the positive electrode active material 100 of one embodiment of the present invention.

However, heat treatment at an excessively high temperature may cause cation mixing, which increases the possibility of entry of the added element such as magnesium into the cobalt sites. Magnesium in the cobalt sites does not have the effect of maintaining the structure belonging to R-3m at the time of high voltage charge. Furthermore, heat treatment at an excessively high temperature might have an adverse effect; for example, cobalt might be reduced to have a valence of two or lithium might be evaporated.

In view of the above, a fluorine compound is preferably added to lithium cobalt oxide before the heat treatment for distributing magnesium throughout the particle. The addition of the fluorine compound decreases the melting point of lithium cobalt oxide. The decreased melting point makes it easier to distribute magnesium throughout the particle at a temperature at which the cation mixing is unlikely to occur. Furthermore, the fluorine compound probably increases corrosion resistance to hydrofluoric acid generated by decomposition of an electrolyte.

When the magnesium concentration is higher than a desired value, the effect of stabilizing a crystal structure becomes small in some cases. This is probably because magnesium enters the cobalt sites in addition to the lithium sites. The number of magnesium atoms in the positive electrode active material of one embodiment of the present invention is preferably greater than or equal to 0.001 times and less than or equal to 0.1 times, further preferably greater than 0.01 times and less than 0.04 times, still further preferably approximately 0.02 times the number of atoms of the transition metal M. Alternatively, it is preferably greater than or equal to 0.001 times and less than 0.04 times. Alternatively, it is preferably greater than or equal to 0.01 times and less than or equal to 0.1 times. The magnesium concentration described here may be a value obtained by element analysis on the whole particles of the positive electrode active material using ICP-MS or the like, or may be a value based on the ratio of the raw materials mixed in the process of forming the positive electrode active material, for example.

As a metal other than cobalt (hereinafter, a metal Z), one or more metals selected from nickel, aluminum, manganese, titanium, vanadium, and chromium may be added to lithium cobalt oxide, for example, and in particular, at least one of nickel and aluminum is preferably added. In some cases, manganese, titanium, vanadium, and chromium are likely to have a valence of four stably and thus contribute highly to a stable structure. The addition of the metal Z may enable the positive electrode active material of one embodiment of the present invention to have a more stable crystal structure in high voltage charged state, for example. Here, in the positive electrode active material of one embodiment of the present invention, the metal Z is preferably added at a concentration that does not greatly change the crystallinity of the lithium cobalt oxide. For example, the metal Z is preferably added at an amount with which the aforementioned Jahn-Teller effect is not exhibited.

As shown in the legend in FIG. 6, aluminum and the transition metal typified by nickel and manganese preferably exist in cobalt sites, but part of them may exist in lithium sites. Magnesium preferably exists in lithium sites. Fluorine may be substituted for part of oxygen.

As the magnesium concentration in the positive electrode active material of one embodiment of the present invention increases, the charge and discharge capacity of the positive electrode active material decreases in some cases. As an example, one possible reason is that the amount of lithium that contributes to charge and discharge decreases when magnesium enters the lithium sites. Another possible reason is that excess magnesium generates a magnesium compound that does not contribute to charge and discharge. When the positive electrode active material of one embodiment of the present invention contains nickel as the metal Z in addition to magnesium, the charge and discharge capacity per weight and per volume can be increased in some cases. When the positive electrode active material of one embodiment of the present invention contains aluminum as the metal Z in addition to magnesium, the charge and discharge capacity per weight and per volume can be increased in some cases. When the positive electrode active material of one embodiment of the present invention contains nickel and aluminum in addition to magnesium, the charge and discharge capacity per weight and per volume can be increased in some cases.

The concentrations of the elements contained in the positive electrode active material of one embodiment of the present invention, such as magnesium and the metal Z, are described below using the number of atoms.

The number of nickel atoms in the positive electrode active material 100 of one embodiment of the present invention is preferably greater than 0% and less than or equal to 7.5%, further preferably greater than or equal to 0.05% and less than or equal to 4%, still further preferably greater than or equal to 0.1% and less than or equal to 2%, and especially preferably greater than or equal to 0.2% and less than or equal to 1% of the number of cobalt atoms. Alternatively, it is preferably greater than 0% and less than or equal to 4%. Alternatively, it is preferably greater than 0% and less than or equal to 2%. Alternatively, it is preferably greater than or equal to 0.05% and less than or equal to 7.5%. Alternatively, it is preferably greater than or equal to 0.05% and less than or equal to 2%. Alternatively, it is preferably greater than or equal to 0.1% and less than or equal to 7.5%. Alternatively, it is preferably greater than or equal to 0.1% and less than or equal to 4%. The nickel concentration described here may be a value obtained by element analysis on the whole particles of the positive electrode active material using GD-MS, ICP-MS, or the like, or may be a value based on the ratio of the raw materials mixed in the process of forming the positive electrode active material, for example.

Nickel contained at any of the above concentrations easily forms a solid solution uniformly throughout the positive electrode active material 100 and thus particularly contributes to stabilization of the crystal structure of the inner portion 100b. When divalent nickel exists in the inner portion 100b, a slight amount of the added element having a valence of two and randomly existing in lithium sites, such as magnesium, might be able to exist more stably in the vicinity of the divalent nickel. Thus, even when high voltage charge and discharge are performed, elution of magnesium might be inhibited. Accordingly, charge and discharge cycle performance might be improved. Such a combination of the effect of nickel in the inner portion 100b and the effect of magnesium, aluminum, titanium, fluorine, or the like in the surface portion 100a extremely effectively stabilizes the crystal structure at the time of high voltage charge.

The number of aluminum atoms in the positive electrode active material of one embodiment of the present invention is preferably greater than or equal to 0.05% and less than or equal to 4%, further preferably greater than or equal to 0.1% and less than or equal to 2%, still further preferably greater than or equal to 0.3% and less than or equal to 1.5% of the number of cobalt atoms. Alternatively, it is preferably greater than or equal to 0.05% and less than or equal to 2%. Alternatively, it is preferably greater than or equal to 0.1% and less than or equal to 4%. The aluminum concentration described here may be a value obtained by element analysis on the whole particles of the positive electrode active material using GD-MS, ICP-MS, or the like, or may be a value based on the ratio of the raw materials mixed in the process of forming the positive electrode active material, for example.

It is preferable that the positive electrode active material of one embodiment of the present invention contain an element Wand phosphorus be used as the element W. The positive electrode active material of one embodiment of the present invention further preferably includes a compound containing phosphorus and oxygen.

When the positive electrode active material of one embodiment of the present invention includes a compound containing the element W, a short circuit can be inhibited while a high voltage charged state is maintained, in some cases.

When the positive electrode active material of one embodiment of the present invention contains phosphorus as the element W, phosphorus may react with hydrogen fluoride generated by the decomposition of the electrolyte, which might decrease the hydrogen fluoride concentration in the electrolyte.

In the case where the electrolyte contains LiPF₆, hydrogen fluoride may be generated by hydrolysis. In some cases, hydrogen fluoride is generated by the reaction of PVDF used as a component of the positive electrode and alkali. The decrease in hydrogen fluoride concentration in the electrolyte may inhibit corrosion of a current collector and/or separation of a coating film or may inhibit a reduction in adhesion properties due to gelling or insolubilization of PVDF.

When containing magnesium in addition to the element W, the positive electrode active material of one embodiment of the present invention is extremely stable in a high voltage charged state. When the element W is phosphorus, the number of phosphorus atoms is preferably greater than or equal to 1% and less than or equal to 20%, further preferably greater than or equal to 2% and less than or equal to 10%, still further preferably greater than or equal to 3% and less than or equal to 8% of the number of cobalt atoms. Alternatively, it is preferably greater than or equal to 1% and less than or equal to and 10%. Alternatively, it is preferably greater than or equal to 1% and less than or equal to 8%. Alternatively, it is preferably greater than or equal to 2% and less than or equal to 20%. Alternatively, it is preferably greater than or equal to 2% and less than or equal to 8%. Alternatively, it is preferably greater than or equal to 3% and less than or equal to 20%. Alternatively, it is preferably greater than or equal to 3% and less than or equal to 10%. In addition, the number of magnesium atoms is preferably greater than or equal to 0.1% and less than or equal to and 10%, further preferably greater than or equal to 0.5% and less than or equal to 5%, still further preferably greater than or equal to 0.7% and less than or equal to 4% of the number of cobalt atoms. Alternatively, it is preferably greater than or equal to 0.1% and less than or equal to 5%. Alternatively, it is preferably greater than or equal to 0.1% and less than or equal to 4%. Alternatively, it is preferably greater than or equal to 0.5% and less than or equal to 10%. Alternatively, it is preferably greater than or equal to 0.5% and less than or equal to 4%. Alternatively, it is preferably greater than or equal to 0.7% and less than or equal to 10%. Alternatively, it is preferably greater than or equal to 0.7% and less than or equal to 5%. The phosphorus concentration and the magnesium concentration described here may each be a value obtained by element analysis on the whole particles of the positive electrode active material using ICP-MS or the like, or may be a value based on the ratio of the raw materials mixed in the process of forming the positive electrode active material, for example.

The positive electrode active material has a crack in some cases. When phosphorus, more specifically, a compound containing phosphorus and oxygen, in the inner portion of the positive electrode active material with the crack on the surface may inhibit crack development, for example.

### <<Surface Portion>>

It is preferable that magnesium be distributed throughout a particle of the positive electrode active material 100 of one embodiment of the present invention, and it is further preferable that the magnesium concentration in the surface portion 100a be higher than the average magnesium concentration in the whole particle. Alternatively, it is preferable that the magnesium concentration in the surface portion 100a be higher than the magnesium concentration in the inner portion 100b. For example, the magnesium concentration in the surface portion 100a measured by XPS or the like is preferably higher than the average magnesium concentration in the whole particle measured by ICP-MS or the like. Alternatively, the magnesium concentration in the surface portion 100a measured by EDX surface analysis or the like is preferably higher than the magnesium concentration in the inner portion 100b.

In the case where the positive electrode active material 100 of one embodiment of the present invention contains an element other than cobalt, for example, one or more metals selected from nickel, aluminum, manganese, iron, and chromium, the concentration of the metal in the surface portion 100a is preferably higher than the average concentration in the whole particle. Alternatively, the concentration of the metal in the surface portion 100a is preferably higher than that in the inner portion 100b. For example, the concentration of the element other than cobalt in the surface portion 100a measured by XPS or the like is preferably higher than the average concentration of the element in the whole particles measured by ICP-MS or the like. Alternatively, the concentration of the element other than cobalt in the surface portion 100a measured by EDX surface analysis or the like is preferably higher than the concentration of the element other than cobalt in the inner portion 100b.

The surface portion 100a is in a state where bonds are cut unlike the inner portion of the crystal, and lithium is extracted from the surface during charge; thus, the lithium concentration in the surface portion 100a tends to be lower than that in the inner portion. Therefore, the surface portion 100a tends to be unstable and its crystal structure is likely to be broken. The higher the magnesium concentration in the surface portion 100a is, the more effectively the change in the crystal structure can be reduced. In addition, a high magnesium concentration in the surface portion 100a probably increases the corrosion resistance to hydrofluoric acid generated by the decomposition of the electrolyte.

The fluorine concentration in the surface portion 100a of the positive electrode active material 100 of one embodiment of the present invention is preferably higher than the average concentration in the whole particle. Alternatively, the fluorine concentration in the surface portion 100a is preferably higher than that in the inner portion 100b. When fluorine exists in the surface portion 100a, which is in contact with the electrolyte, the corrosion resistance to hydrofluoric acid can be effectively increased.

As described above, the surface portion 100a of the positive electrode active material 100 of one embodiment of the present invention preferably has a composition different from that in the inner portion 100b, i.e., the concentrations of the added elements such as magnesium and fluorine are preferably higher than those in the inner portion 100b. The composition preferably has a crystal structure stable at room temperature (25° C.). Accordingly, the surface portion 100a may have a crystal structure different from that of the inner portion 100b. For example, at least part of the surface portion 100a of the positive electrode active material 100 of one embodiment of the present invention may have the rock-salt crystal structure. When the surface portion 100a and the inner portion 100b have different crystal structures, the orientations of crystals in the surface portion 100a and the inner portion 100b are preferably substantially aligned with each other.

Anions of a layered rock-salt crystal and anions of a rock-salt crystal form a cubic close-packed structure (face-centered cubic lattice structure). Anions of an O3′ type crystal are presumed to form a cubic close-packed structure.

Note that in this specification and the like, a structure is referred to as a cubic close-packed structure when three layers of anions are shifted and stacked like “ABCABC” in the structure. Accordingly, anions do not necessarily form a cubic lattice structure. At the same time, actual crystals always have a defect and thus, analysis results are not necessarily consistent with the theory. For example, in an electron diffraction pattern or an FFT (fast Fourier transform) pattern of a TEM image or the like, a spot may appear in a position slightly different from a theoretical position. For example, anions may be regarded as forming a cubic close-packed structure when a difference in orientation from a theoretical position is 5° or less or 2.5° or less.

When a layered rock-salt crystal and a rock-salt crystal are in contact with each other, there is a crystal plane at which orientations of cubic close-packed structures formed of anions are aligned with each other.

The description can also be made as follows. An anion on the (111) plane of a cubic crystal structure has a triangular arrangement. A layered rock-salt structure, which belongs to the space group R-3m and is a rhombohedral structure, is generally represented by a composite hexagonal lattice for easy understanding of the structure, and the (0001) plane of the layered rock-salt structure has a hexagonal lattice. The triangle lattice on the (111) plane of the cubic crystal has atomic arrangement similar to that of the hexagonal lattice on the (0001) plane of the layered rock-salt structure. These lattices being consistent with each other can be expressed as “orientations of the cubic close-packed structures are aligned with each other”.

Note that a space group of the layered rock-salt crystal and the O3′ type crystal is R-3m, which is different from the space groups Fm-3m (the space group of a general rock-salt crystal) and the space group Fd-3m of rock-salt crystals; thus, the Miller index of the crystal plane satisfying the above conditions in the layered rock-salt crystal and the O3′ type crystal is different from that in the rock-salt crystal. In this specification, in the layered rock-salt crystal, the O3′ type crystal, and the rock-salt crystal, a state where the orientations of the cubic close-packed structures formed of anions are aligned with each other may be referred to as a state where crystal orientations are substantially aligned with each other.

The orientations of crystals in two regions being substantially aligned with each other can be judged, for example, from a TEM (transmission electron microscope) image, a STEM (scanning transmission electron microscope) image, a HAADF-STEM (high-angle annular dark-field scanning TEM) image, an ABF-STEM (annular bright-field scanning transmission electron microscope) image, an electron diffraction pattern, or an FFT pattern of a TEM image or the like. X-ray diffraction (XRD), neutron diffraction, or the like can also be used for judging.

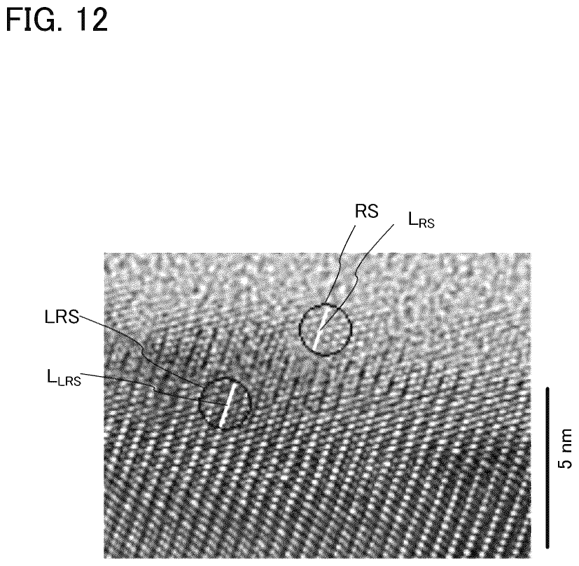

FIG. 12 shows an example of a TEM image in which orientations of a layered rock-salt crystal LRS and a rock-salt crystal RS are substantially aligned with each other. In a TEM image, a STEM image, a HAADF-STEM image, an ABF-STEM image, and the like, an image reflecting a crystal structure is obtained.

For example, in a high-resolution TEM image, a contrast derived from a crystal plane is obtained. When an electron beam is incident perpendicularly to the c-axis of a composite hexagonal lattice of a layered rock-salt structure, for example, a contrast derived from the (0003) plane is obtained as repetition of bright lines and dark lines because of diffraction and interference of the electron beam. Thus, when repetition of bright lines and dark lines is observed and the angle between the bright lines (e.g., L_{RS} and L_{LRS} in FIG. 12) is 5° or less or 2.5° or less in the TEM image, it can be judged that the crystal planes are substantially aligned with each other, that is, orientations of the crystals are substantially aligned with each other. Similarly, when the angle between the dark lines is 5° or less or 2.5° or less, it can be judged that orientations of the crystals are substantially aligned with each other.

In a HAADF-STEM image, a contrast corresponding to the atomic number is obtained, and an element having a larger atomic number is observed to be brighter. For example, in the case of lithium cobalt oxide that has a layered rock-salt structure belonging to the space group R-3m, cobalt (atomic number: 27) has the largest atomic number; hence, an electron beam is strongly scattered at the position of a cobalt atom, and arrangement of the cobalt atoms is observed as bright lines or arrangement of high-luminance dots. Thus, when the lithium cobalt oxide having a layered rock-salt crystal structure is observed perpendicularly to the c-axis, arrangement of the cobalt atoms is observed as bright lines or arrangement of high-luminance dots, and arrangement of lithium atoms and oxygen atoms is observed as dark lines or a low-luminance region in the direction perpendicular to the c-axis. The same applies to the case where fluorine (atomic number: 9) and magnesium (atomic number: 12) are included as the added elements of the lithium cobalt oxide.

Consequently, in the case where repetition of bright lines and dark lines is observed in two regions having different crystal structures and the angle between the bright lines is 5° or less or 2.5° or less in a HAADF-STEM image, it can be judged that arrangements of the atoms are substantially aligned with each other, that is, orientations of the crystals are substantially aligned with each other. Similarly, when the angle between the dark lines is 5° or less or 2.5° or less, it can be judged that orientations of the crystals are substantially aligned with each other.

With an ABF-STEM, an element having a smaller atomic number is observed to be brighter, but a contrast corresponding to the atomic number is obtained as with a HAADF-STEM; hence, in an ABF-STEM image, crystal orientations can be judged as in a HAADF-STEM image.

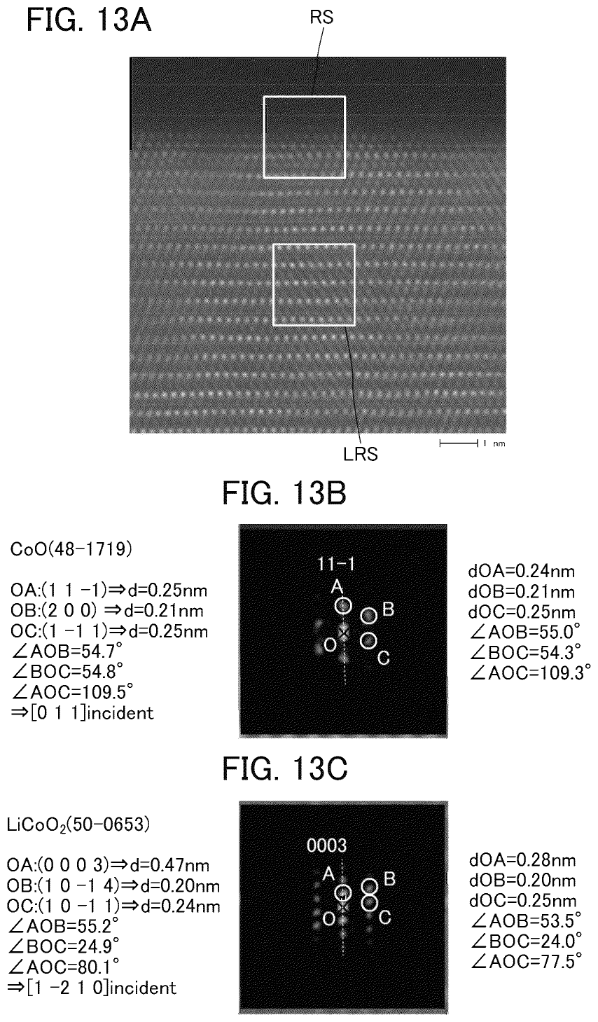

FIG. 13A shows an example of a STEM image in which orientations of the layered rock-salt crystal LRS and the rock-salt crystal RS are substantially aligned with each other. FIG. 13B shows an FFT pattern of a region of the rock-salt crystal RS, and FIG. 13C shows an FFT pattern of a region of the layered rock-salt crystal LRS. In FIG. 13B and FIG. 13C, the composition, the JCPDS card number, and d values and angles to be calculated are shown on the left. The measured values are shown on the right. A spot denoted by O is zero-order diffraction.

A spot denoted by A in FIG. 13B is derived from 11−1 reflection of a cubic structure. A spot denoted by A in FIG. 13C is derived from 0003 reflection of a layered rock-salt structure. It is found from FIG. 13B and FIG. 13C that the direction of the 11−1 reflection of the cubic structure and the direction of the 0003 reflection of the layered rock-salt structure are substantially aligned with each other. That is, a straight line that passes through AO in FIG. 13B is substantially parallel to a straight line that passes through AO in FIG. 13C. Here, the terms “substantially aligned” and “substantially parallel” mean that the angle between the two is 5° or less or 2.5° or less.

When the orientations of the layered rock-salt crystal and the rock-salt crystal are substantially aligned with each other in the above manner in an FFT pattern and electron diffraction, the <0003> orientation of the layered rock-salt crystal and the <11−1> orientation of the rock-salt crystal may be substantially aligned with each other. In that case, it is preferable that these reciprocal lattice points be spot-shaped, that is, they be not connected to other reciprocal lattice points. The state where reciprocal lattice points are spot-shaped and not connected to other reciprocal lattice points means high crystallinity.

When the direction of the 11−1 reflection of the cubic structure and the direction of the 0003 reflection of the layered rock-salt structure are substantially aligned with each other as described above, a spot that is not derived from the 0003 reflection of the layered rock-salt structure may be observed, depending on the incident direction of the electron beam, on a reciprocal lattice space different from the direction of the 0003 reflection of the layered rock-salt structure. For example, a spot denoted by B in FIG. 13C is derived from 1014 reflection of the layered rock-salt structure. This is sometimes observed at a position where the difference in orientation from the reciprocal lattice point derived from the 0003 reflection of the layered rock-salt structure (A in FIG. 13C) is greater than or equal to 520 and less than or equal to 560 (i.e., ∠AOB is 52° to 56°) and d is greater than or equal to 0.19 nm and less than or equal to 0.21 nm. Note that these indices are just an example, and the spot does not necessarily correspond with them and may be, for example, a direction equivalent to the indices.

Similarly, a spot that is not derived from the 11−1 reflection of the cubic structure may be observed on a reciprocal lattice space different from the direction where the 11−1 reflection of the cubic structure is observed. For example, a spot denoted by B in FIG. 13B is derived from 200 reflection of the cubic structure. A diffraction spot is sometimes observed at a position where the difference in orientation from the spot derived from the 11−1 reflection of the cubic structure (A in FIG. 13B) is greater than or equal to 540 and less than or equal to 560 (i.e., ∠AOB is 54° to 56°). Note that these indices are just an example, and the spot does not necessarily correspond with them and may be, for example, a direction equivalent to the indices.

It is known that in a layered rock-salt positive electrode active material, such as lithium cobalt oxide, the (0003) plane and a plane equivalent thereto and the (10-14) plane and a plane equivalent thereto are likely to be crystal planes. Thus, a sample to be observed can be processed to be thin by FIB or the like such that an electron beam of a TEM, for example, enters in [12-10], in order to easily observe the (0003) plane in careful observation of the shape of the positive electrode active material with a SEM or the like. To judge alignment of crystal orientations, a sample is preferably processed to be thin so that the (0003) plane of the layered rock-salt structure is easily observed.

However, in the surface portion 100a where only MgO is contained or MgO and CoO(II) form a solid solution, it is difficult to insert and extract lithium. Thus, the surface portion 100a should contain at least cobalt, and also contain lithium in a discharged state to have the path through which lithium is inserted and extracted. The cobalt concentration is preferably higher than the magnesium concentration.

The added element X is preferably positioned in the surface portion 100a of the positive electrode active material 100 of one embodiment of the present invention. For example, the positive electrode active material 100 of one embodiment of the present invention may be covered with the coating film containing the added element X.

### <<Grain Boundary>>

It is further preferable that the added element contained in the positive electrode active material 100 of one embodiment of the present invention have the above-described distribution and be partly segregated in the crystal grain boundary 101.

Specifically, the magnesium concentration at the crystal grain boundary 101 and the vicinity thereof in the positive electrode active material 100 is preferably higher than that in the other regions in the inner portion 100b. In addition, the fluorine concentration at the crystal grain boundary 101 and the vicinity thereof is preferably higher than that in the other regions in the inner portion 100b.

The crystal grain boundary 101 is a plane defect, and thus tends to be unstable and suffer a change in the crystal structure like the surface of the particle. Thus, the higher the magnesium concentration at the crystal grain boundary 101 and the vicinity thereof is, the more effectively the change in the crystal structure can be reduced.

When the magnesium concentration and the fluorine concentration are high at the crystal grain boundary 101 and the vicinity thereof, the magnesium concentration and the fluorine concentration in the vicinity of a surface generated by a crack are also high even when the crack is generated along the crystal grain boundary 101 of the positive electrode active material 100 of one embodiment of the present invention. Thus, the positive electrode active material including a crack can also have an increased corrosion resistance to hydrofluoric acid.

Note that in this specification and the like, the vicinity of the crystal grain boundary 101 refers to a region of approximately 10 nm from the grain boundary. The crystal grain boundary refers to a plane where atomic arrangement is changed and which can be observed with an electron microscope. Specifically, the crystal grain boundary refers to a portion where the angle formed by repetition of bright lines and dark lines in an electron microscope image exceeds 5° or a portion where a crystal structure cannot be observed in an electron microscope image.

### <<Particle Diameter>>

When the particle diameter of the positive electrode active material 100 of one embodiment of the present invention is too large, there are problems such as difficulty in lithium diffusion and large surface roughness of an active material layer at the time when the material is applied to a current collector. By contrast, too small a particle diameter causes problems such as difficulty in loading of the active material layer at the time when the material is applied to the current collector and overreaction with the electrolyte. Therefore, the median diameter (D50) is preferably greater than or equal to 1 μm and less than or equal to 100 μm, further preferably greater than or equal to 2 μm and less than or equal to 40 μm, still further preferably greater than or equal to 5 μm and less than or equal to 30 μm. Alternatively, it is preferably greater than or equal to 1 m and less than or equal to 40 μm. Alternatively, it is preferably greater than or equal to 1 μm and less than or equal to 30 μm. Alternatively, it is preferably greater than or equal to 2 μm and less than or equal to 100 μm. Alternatively, it is preferably greater than or equal to 2 μm and less than or equal to 30 μm. Alternatively, it is preferably greater than or equal to 5 μm and less than or equal to 100 μm. Alternatively, it is preferably greater than or equal to 5 μm and less than or equal to 40 μm.

### <Analysis Method>

Whether or not a given positive electrode active material is the positive electrode active material 100 of one embodiment of the present invention, which has the O3′ type crystal structure at the time of high voltage charge, can be judged by analyzing a high voltage charged positive electrode by XRD, electron diffraction, neutron diffraction, electron spin resonance (ESR), nuclear magnetic resonance (NMR), or the like. XRD is particularly preferable because the symmetry of a transition metal such as cobalt in the positive electrode active material can be analyzed with high resolution, comparison of the degree of crystallinity and comparison of the crystal orientation can be performed, distortion of lattice periodicity and the crystallite size can be analyzed, and a positive electrode obtained only by disassembling a secondary battery can be measured with sufficient accuracy, for example.

As described above, the positive electrode active material 100 of one embodiment of the present invention features in a small change in the crystal structure between a high voltage charged state and a discharged state. A material in which 50 wt % or more of the crystal structure largely changes between a high voltage charged state and a discharged state is not preferable because the material cannot withstand high voltage charge and discharge. It should be noted that the intended crystal structure is not obtained in some cases only by addition of the added element. For example, although the positive electrode active material that is lithium cobalt oxide containing magnesium and fluorine is a commonality, the positive electrode active material has the O3′ type crystal structure at 60 wt % or more in some cases, and has the H1-3 type crystal structure at 50 wt % or more in other cases, when charged at a high voltage. Furthermore, the positive electrode active material has the O3′ type crystal structure at almost 100 wt % at a predetermined voltage, and increasing the voltage to be higher than the predetermined voltage may cause the H1-3 type crystal structure. Thus, to determine whether or not a positive electrode active material is the positive electrode active material 100 of one embodiment of the present invention, the crystal structure should be analyzed by XRD and other methods.

However, the crystal structure of a positive electrode active material in a high voltage charged state or a discharged state may be changed with exposure to the air. For example, the O3′ type crystal structure changes into the H1-3 type structure in some cases. For that reason, all samples are preferably handled in an inert atmosphere such as an argon atmosphere.

### <<Charging Method>>

High voltage charge for determining whether or not a composite oxide is the positive electrode active material 100 of one embodiment of the present invention can be performed on a coin cell (CR2032 type with a diameter of 20 mm and a height of 3.2 mm) with a lithium counter electrode, for example

More specifically, a positive electrode can be formed by application of a slurry in which the positive electrode active material, a conductive additive, and a binder are mixed to a positive electrode current collector made of aluminum foil.

A lithium metal can be used for a counter electrode. Note that when the counter electrode is formed using a material other than the lithium metal, the potential of a secondary battery differs from the potential of the positive electrode. Unless otherwise specified, the voltage and the potential in this specification and the like refer to the potential of a positive electrode.

As a lithium salt contained in an electrolyte, 1 mol/L lithium hexafluorophosphate (LiPF₆) can be used, and as an organic solvent, a solution in which ethylene carbonate (EC) and diethyl carbonate (DEC) at a volume ratio of 3:7 and vinylene carbonate (VC) at 2 wt % are mixed can be used.

As a separator, 25-μm-thick polypropylene can be used.

Stainless steel (SUS) can be used for a positive electrode can and a negative electrode can.

The coin cell fabricated with the above conditions is subjected to constant current charge at 0.5 C to a freely selected voltage (e.g., 4.6 V, 4.65 V, or 4.7 V) and then constant voltage charge until the current value reaches 0.01 C. Note that 1 C can be 137 mA/g or 200 mA/g. The temperature is set to 25° C. After the charge is performed in this manner, the coin cell is disassembled in a glove box with an argon atmosphere to take out the positive electrode, whereby the high voltage charge positive electrode active material can be obtained. In order to inhibit a reaction with components in the external environment, the positive electrode is preferably enclosed in an argon atmosphere in performing various analyses later. For example, XRD can be performed on the positive electrode enclosed in an airtight container with an argon atmosphere.

### <<XRD>>

The apparatus and conditions for the XRD measurement are not particularly limited. The measurement can be performed with the apparatus and conditions as described below, for example.
XRD apparatus: D8 ADVANCE produced by Bruker AXS
X-ray source: CuKα₁ radiation
Output: 40 kV, 40 mA
Slit system: Div. Slit, 0.5°
Detector: LynxEye
Scanning method: 2θ/θ continuous scanning
Measurement range (2θ): from 150 to 900
Step width (2θ): 0.01°
Counting time: 1 second/step
Rotation of sample stage: 15 rpm

In the case where the measurement sample is a powder, the sample can be set by, for example, being put in a glass sample holder or being sprinkled on a reflection-free silicon plate to which grease is applied. In the case where the measurement sample is a positive electrode, the sample can be set in such a manner that the positive electrode is attached to a substrate with a double-sided adhesive tape so that the position of the positive electrode active material layer can be adjusted to the measurement plane required by the apparatus.

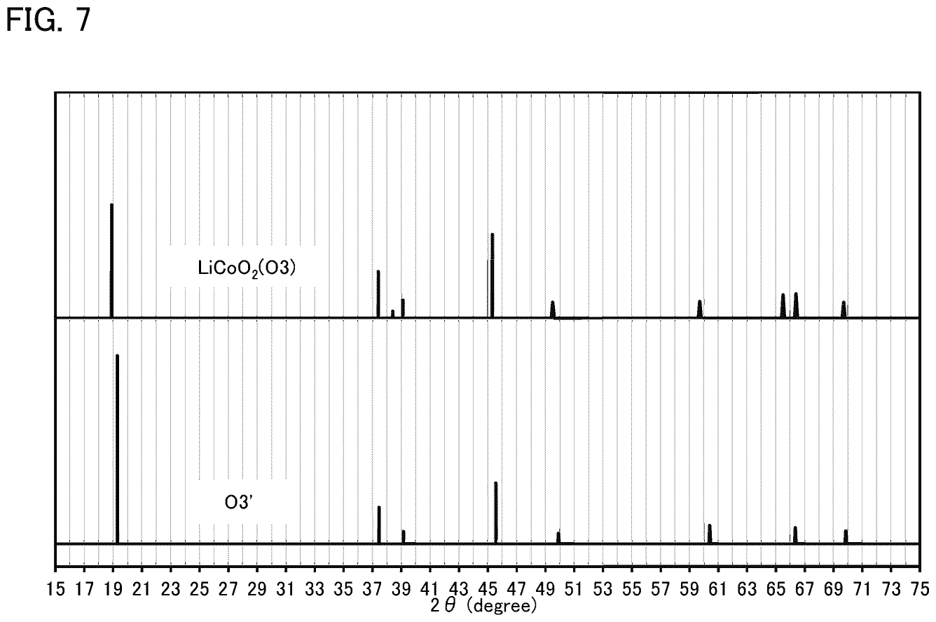

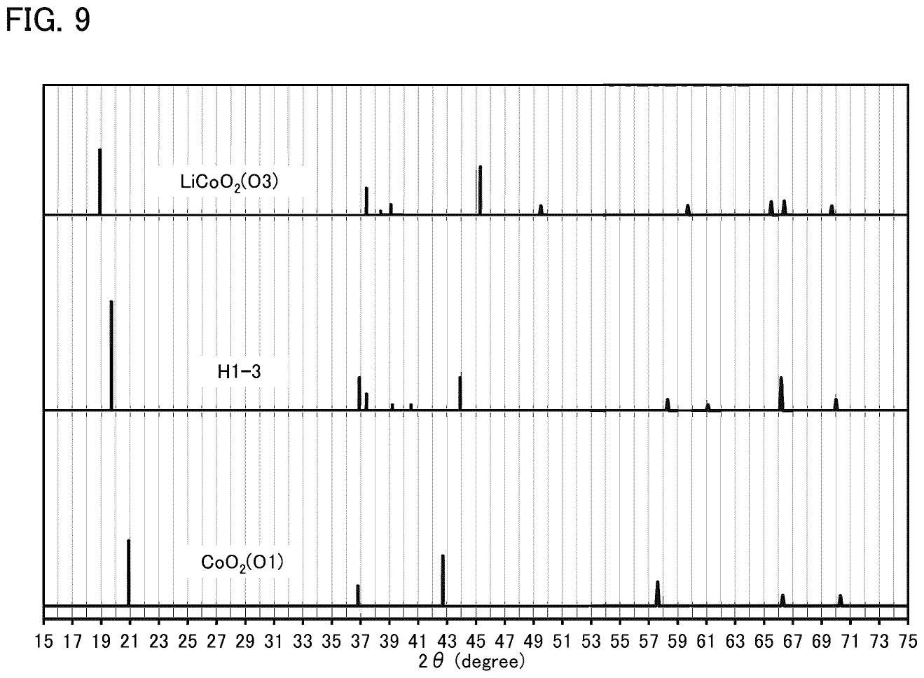

FIG. 7 and FIG. 9 show ideal powder XRD patterns with CuKα₁ radiation that are calculated from models of the O3′ type crystal structure and the H1-3 type crystal structure. For comparison, ideal XRD patterns calculated from the crystal structure of LiCoO₂ (O3) with a charge depth of 0 and the crystal structure of CoO₂ (O1) with a charge depth of 1 are also shown. Note that the patterns of LiCoO₂ (O3) and CoO₂ (O1) are made from crystal structure data obtained from the ICSD (Inorganic Crystal Structure Database) (see Non-Patent Document 3) with Reflex Powder Diffraction, which is a module of Materials Studio (BIOVIA). The range of 20 is from 15° to 75°, the step size is 0.01, the wavelength λ1 is 1.540562×10⁻¹⁰ m, the wavelength 12 is not set, and a single monochromator is used. The pattern of the H1-3 type crystal structure is similarly made from the crystal structure data disclosed in Non-Patent Document 2. The O3′ type crystal structure is estimated from the XRD pattern of the positive electrode active material of one embodiment of the present invention, the crystal structure is fitted with TOPAS Ver. 3 (crystal structure analysis software produced by Bruker Corporation), and the XRD pattern of the O3′ type crystal structure is made in a manner similar to that for other structures.

As shown in FIG. 7, the O3′ type crystal structure exhibits diffraction peaks at 2θ of 19.30±0.20° (greater than or equal to 19.100 and less than or equal to 19.50°) and 2θ of 45.55±0.100 (greater than or equal to 45.450 and less than or equal to 45.65°). More specifically, the O3′ type crystal structure exhibits sharp diffraction peaks at 2θ of 19.30±0.100 (greater than or equal to 19.200 and less than or equal to 19.40°) and 2θ of 45.55±0.05° (greater than or equal to 45.500 and less than or equal to 45.60°). By contrast, as shown in FIG. 9, the H1-3, LiCoO₂ (O3), and CoO₂ (P-3 ml, O1) do not exhibit peaks at these positions. Thus, the peaks at 2θ of 19.30±0.20° and 2θ of 45.55±0.10° in a high voltage charged state can be the features of the positive electrode active material 100 of one embodiment of the present invention.

It can be said that the positions of the XRD diffraction peaks exhibited by the crystal structure with a charge depth of 0 are close to those of the XRD diffraction peaks exhibited by the crystal structure at the time of high voltage charge. More specifically, it can be said that a difference in the positions of two or more, preferably three or more of the main diffraction peaks between the crystal structures is 20=0.7° or less, preferably 20=0.5° or less.

Although the positive electrode active material 100 of one embodiment of the present invention has the O3′ type crystal structure at the time of high voltage charge, not all the particles necessarily have the O3′ type crystal structure. Some of the particles may have another crystal structure or be amorphous. Note that when the XRD patterns are subjected to the Rietveld analysis, the O3′ type crystal structure preferably accounts for greater than or equal to 50 wt %, further preferably greater than or equal to 60 wt %, still further preferably greater than or equal to 66 wt %. The positive electrode active material in which the O3′ type crystal structure accounts for greater than or equal to 50 wt %, preferably greater than or equal to 60 wt %, further preferably greater than or equal to 66 wt % can have sufficiently good cycle performance.

Furthermore, even after 100 or more cycles of charge and discharge after the measurement starts, the O3′ type crystal structure preferably accounts for greater than or equal to 35 wt %, further preferably greater than or equal to 40 wt %, still further preferably greater than or equal to 43 wt %, in the Rietveld analysis.

The crystallite size of the O3′ type crystal structure of the positive electrode active material particle is only decreased to approximately one-tenth that of LiCoO₂ (O3) in a discharged state. Thus, the peak of the O3′ type crystal structure can be clearly observed after high voltage charge even under the same XRD measurement conditions as those of a positive electrode before charge and discharge. By contrast, simple LiCoO₂ has a small crystallite size and exhibits a broad and small peak although it can partly have a structure similar to the O3′ type crystal structure. The crystallite size can be calculated from the half width of the XRD peak.

As described above, the influence of the Jahn-Teller effect is preferably small in the positive electrode active material of one embodiment of the present invention. It is preferable that the positive electrode active material of one embodiment of the present invention have a layered rock-salt crystal structure and mainly contain cobalt as a transition metal. The positive electrode active material of one embodiment of the present invention may contain the above-described metal Z in addition to cobalt as long as the influence of the Jahn-Teller effect is small.

The range of the lattice constants where the influence of the Jahn-Teller effect is presumed to be small in the positive electrode active material is examined by XRD analysis.

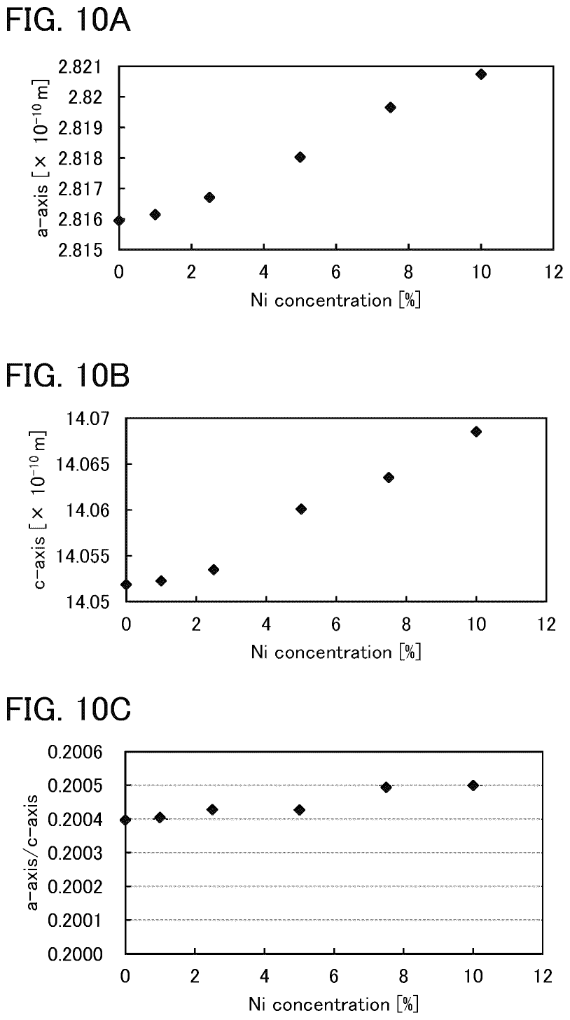

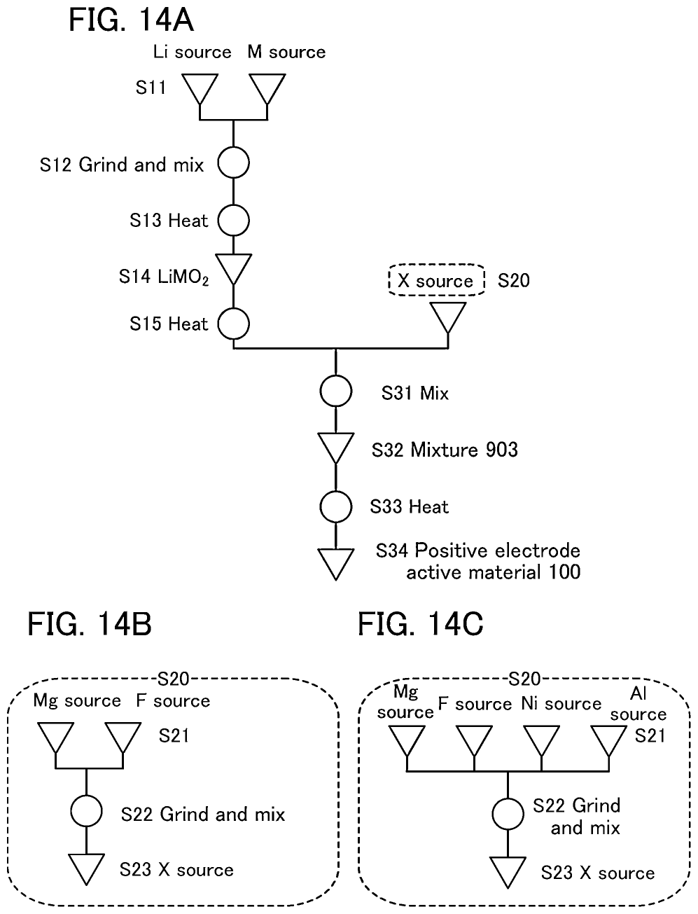

FIG. 10 shows the calculation results of the lattice constants of the a-axis and the c-axis by XRD in the case where the positive electrode active material of one embodiment of the present invention has a layered rock-salt crystal structure and contains cobalt and nickel. FIG. 10A shows the results of the a-axis, and FIG. 10B shows the results of the c-axis. Note that the XRD patterns of a powder after the synthesis of the positive electrode active material before incorporation into a positive electrode are used for the calculation. The nickel concentration on the horizontal axis represents a nickel concentration with the sum of cobalt atoms and nickel atoms assumed as 100%. The positive electrode active material is formed in accordance with the formation method in FIG. 14 described later except that heating in Step S15 is not performed.

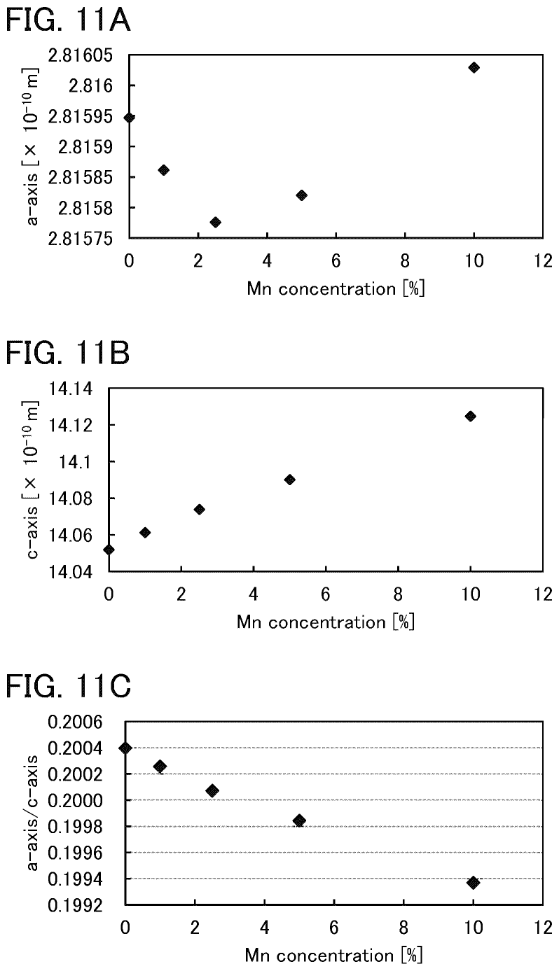

FIG. 11 shows the estimation results of the lattice constants of the a-axis and the c-axis by XRD in the case where the positive electrode active material of one embodiment of the present invention has a layered rock-salt crystal structure and contains cobalt and manganese. FIG. 11A shows the results of the a-axis, and FIG. 11B shows the results of the c-axis. Note that the lattice constants shown in FIG. 11 are obtained by XRD measurement of a powder after the synthesis of the positive electrode active material before incorporation into a positive electrode. The manganese concentration on the horizontal axis represents a manganese concentration with the sum of cobalt atoms and manganese atoms assumed as 100%. The positive electrode active material is formed in accordance with the formation method of FIG. 14 described later except that heating in Step S15 is not performed.

FIG. 10C shows values obtained by dividing the lattice constants of the a-axis by the lattice constants of the c-axis (a-axis/c-axis) in the positive electrode active material, whose results of the lattice constants are shown in FIG. 10A and FIG. 10B. FIG. 11C shows values obtained by dividing the lattice constants of the a-axis by the lattice constants of the c-axis (a-axis/c-axis) in the positive electrode active material, whose results of the lattice constants are shown in FIG. 11A and FIG. 11B.

As shown in FIG. 10C, the value of a-axis/c-axis tends to significantly change between nickel concentrations of 5% and 7.5%, and the distortion of the a-axis becomes large at a nickel concentration of 7.5%. This distortion may be the Jahn-Teller distortion. It is suggested that an excellent positive electrode active material with small Jahn-Teller distortion can be obtained at a nickel concentration of lower than 7.5%.

FIG. 11A indicates that the lattice constant changes differently at a manganese concentration of 5% or higher and does not follow the Vegard's law. This suggests that the crystal structure changes at a manganese concentration of 5% or higher. Thus, the manganese concentration is preferably 4% or lower, for example.

Note that the nickel concentration and the manganese concentration in the surface portion 100a are not limited to the above ranges. In other words, the nickel concentration and the manganese concentration in the surface portion 100a may be higher than the above concentrations in some cases.

Preferable ranges of the lattice constants of the positive electrode active material of one embodiment of the present invention are examined above. In the layered rock-salt crystal structure of the particle of the positive electrode active material in a discharged state or a state where charge and discharge are not performed, which can be estimated from the XRD patterns, the a-axis lattice constant is preferably greater than 2.814×10⁻¹⁰ m and less than 2.817×10⁻¹⁰ m, and the c-axis lattice constant is preferably greater than 14.05×10⁻¹⁰ m and less than 14.07×10⁻¹⁰ m. The state where charge and discharge are not performed may be the state of a powder before the formation of a positive electrode of a secondary battery.

Alternatively, in the layered rock-salt crystal structure of particle of the positive electrode active material in the discharged state or the state where charge and discharge are not performed, the value obtained by dividing the a-axis lattice constant by the c-axis lattice constant (a-axis/c-axis) is preferably greater than 0.20000 and less than 0.20049.

Alternatively, when the layered rock-salt crystal structure of the particle of the positive electrode active material in the discharged state or the state where charge and discharge are not performed is subjected to XRD analysis, a first peak is observed at 20 of greater than or equal to 18.500 and less than or equal to 19.30°, and a second peak is observed at 20 of greater than or equal to 38.000 and less than or equal to 38.80°, in some cases.

Note that the peaks appearing in the powder XRD patterns reflect the crystal structure of the inner portion 100b of the positive electrode active material 100, which accounts for the majority of the volume of the positive electrode active material 100. The crystal structure of the surface portion 100a, the crystal grain boundary 101, or the like can be analyzed by electron diffraction of a cross section of the positive electrode active material 100, for example.

### <<XPS>>

A region that is approximately 2 to 8 nm (normally, less than or equal to 5 nm) in depth from a surface can be analyzed by X-ray photoelectron spectroscopy (XPS); thus, the concentrations of elements in approximately half the depth of the surface portion 100a can be quantitatively analyzed. The bonding states of the elements can be analyzed by narrow scanning. Note that the quantitative accuracy of XPS is approximately ±1 atomic % in many cases. The lower detection limit is approximately 1 atomic % but depends on the element.

When the positive electrode active material 100 of one embodiment of the present invention is subjected to XPS analysis, the number of atoms of the added element is preferably greater than or equal to 1.6 times and less than or equal to 6.0 times, further preferably greater than or equal to 1.8 times and less than 4.0 times the number of atoms of the transition metal M. When the added element is magnesium and the transition metal M is cobalt, the number of magnesium atoms is preferably greater than or equal to 1.6 times and less than or equal to 6.0 times, further preferably greater than or equal to 1.8 times and less than 4.0 times the number of cobalt atoms. The number of atoms of a halogen such as fluorine is preferably greater than or equal to 0.2 times and less than or equal to 6.0 times, further preferably greater than or equal to 1.2 times and less than or equal to 4.0 times the number of atoms of the transition metal M.

In the XPS analysis, monochromatic aluminum can be used as an X-ray source, for example. An extraction angle is, for example, 45°. For example, the measurement can be performed using the following apparatus and conditions.
Measurement device: Quantera II produced by PHI, Inc.
X-ray source: monochromatic Al (1486.6 eV)
Detection area: 100 μm ϕ
Detection depth: approximately 4 to 5 nm (extraction angle 45°)
Measurement spectrum: wide scanning, narrow scanning of each detected element

In addition, when the positive electrode active material 100 of one embodiment of the present invention is analyzed by XPS, a peak indicating the bonding energy of fluorine with another element is preferably at greater than or equal to 682 eV and less than 685 eV, further preferably approximately 684.3 eV. This bonding energy is different from that of lithium fluoride (685 eV) and that of magnesium fluoride (686 eV). That is, the positive electrode active material 100 of one embodiment of the present invention containing fluorine is preferably in the bonding state other than lithium fluoride and magnesium fluoride.

Furthermore, when the positive electrode active material 100 of one embodiment of the present invention is analyzed by XPS, a peak indicating the bonding energy of magnesium with another element is preferably at greater than or equal to 1302 eV and less than 1304 eV, further preferably approximately 1303 eV. This bonding energy is different from that of magnesium fluoride (1305 eV) and is close to that of magnesium oxide. That is, the positive electrode active material 100 of one embodiment of the present invention containing magnesium is preferably in the bonding state other than magnesium fluoride.

The concentrations of the added elements that preferably exist in the surface portion 100a in a large amount, such as magnesium and aluminum, measured by XPS or the like are preferably higher than the concentrations measured by ICP-MS (inductively coupled plasma mass spectrometry), GD-MS (glow discharge mass spectrometry), or the like.

When a cross section of the positive electrode active material 100 is exposed by processing and analyzed by TEM-EDX, the concentrations of magnesium and aluminum in the surface portion 100a are preferably higher than those in the inner portion 100b. An FIB (Focused Ion Beam) can be used for the processing, for example.

In the XPS (X-ray photoelectron spectroscopy) analysis, the number of magnesium atoms is preferably greater than or equal to 0.4 times and less than or equal to 1.5 times the number of cobalt atoms. In the ICP-MS analysis, the atomic ratio of magnesium to cobalt (Mg/Co) is preferably greater than or equal to 0.001 and less than or equal to 0.06.

By contrast, it is preferable that nickel, which is one of the transition metals M, not be unevenly distributed in the surface portion 100a but be distributed in the entire positive electrode active material 100. Note that one embodiment of the present invention is not limited thereto in the case where the above-described region where the added element is unevenly distributed exists.

### <<ESR>>

As described above, the positive electrode active material of one embodiment of the present invention preferably contains cobalt and nickel as the transition metal and magnesium as the added element. It is preferable that Ni²⁺ be substituted for part of Co³⁺ and Mg²⁺ be substituted for part of Li⁺ accordingly. Accompanying the substitution of Mg²⁺ for Li⁺, the Ni²⁺ might be reduced to be Ni³⁺. Accompanying the substitution of Mg²⁺ for part of Li⁺, Co³⁺ in the vicinity of Mg²⁺ might be reduced to be Co²⁺. Accompanying the substitution of Mg²⁺ for part of Co³⁺, Co³⁺ in the vicinity of Mg²⁺ might be oxidized to be Co⁴⁺.

Thus, the positive electrode active material of one embodiment of the present invention preferably contains one or more of Ni²⁺, Ni³⁺, Co²⁺, and Co⁴⁺. Moreover, the spin density attributed to one or more of Ni²⁺, Ni³⁺, Co²⁺, and Co⁴⁺ per weight of the positive electrode active material is preferably higher than or equal to 2.0×10¹⁷ spins/g and less than or equal to 1.0×10²¹ spins/g. The positive electrode active material preferably has the above spin density, in which case the crystal structure can be stable particularly in a charged state. Note that too high a magnesium concentration might reduce the spin density attributed to one or more of Ni²⁺, Ni³⁺, Co²⁺, and Co⁴⁺.

The spin density of a positive electrode active material can be analyzed by electron spin resonance (ESR), for example.

### <<EPMA>>

Quantitative analysis of elements can be conducted by EPMA (electron probe microanalysis). In surface analysis, distribution of each element can be analyzed.

In EPMA, a region from a surface to a depth of approximately 1 μm is analyzed. Thus, the concentration of each element is sometimes different from measurement results obtained by other analysis methods. For example, when surface analysis is performed on the positive electrode active material 100, the concentration of the added element existing in the surface portion might be lower than the concentration obtained in XPS. The concentration of the added element existing in the surface portion might be higher than the concentration obtained in ICP-MS or a value based on the ratio of the raw materials mixed in the process of forming the positive electrode active material.

EPMA surface analysis of a cross section of the positive electrode active material 100 of one embodiment of the present invention preferably reveals a concentration gradient in which the concentration of the added element increases from the inner portion toward the surface portion. Specifically, each of magnesium, fluorine, titanium, and silicon preferably has a concentration gradient in which the concentration increases from the inner portion toward the surface as shown in FIG. 2B1 and FIG. 2C1. The concentration of aluminum preferably has a peak in a region deeper than the region where the concentration of any of the above elements has a peak, as shown in FIG. 2B2 and FIG. 2C2. The aluminum concentration peak may be located in the surface portion or located deeper than the surface portion.

Note that the surface and the surface portion of the positive electrode active material of one embodiment of the present invention do not contain a carbonate, a hydroxy group, or the like which is chemisorbed after formation of the positive electrode active material. Furthermore, an electrolyte, a binder, a conductive material, and a compound originating from any of these that are attached to the surface of the positive electrode active material are not contained either. Thus, in quantitative analysis of the elements contained in the positive electrode active material, correction may be performed to exclude carbon, hydrogen, excess oxygen, excess fluorine, and the like that might be detected in surface analysis such as XPS and EPMA.

### <<Surface Roughness and Specific Surface Area>>

The positive electrode active material 100 of one embodiment of the present invention preferably has a smooth surface with little unevenness. A smooth surface with little unevenness indicates favorable distribution of the added element in the surface portion 100a.

A smooth surface with little unevenness can be recognized from, for example, a cross-sectional SEM image or a cross-sectional TEM image of the positive electrode active material 100 or the specific surface area of the positive electrode active material 100.

The level of the surface smoothness of the positive electrode active material 100 can be quantified from its cross-sectional SEM image, as described below, for example.

First, the positive electrode active material 100 is processed with an FIB or the like such that its cross section is exposed. At this time, the positive electrode active material 100 is preferably covered with a protective film, a protective agent, or the like. Next, a SEM image of the interface between the positive electrode active material 100 and the protective film or the like is taken. The SEM image is subjected to noise processing using image processing software. For example, the Gaussian Blur (σ=2) is performed, followed by binarization. In addition, interface extraction is performed using image processing software. Moreover, an interface line between the positive electrode active material 100 and the protective film or the like is selected with an automatic selection tool or the like, and data is extracted to spreadsheet software or the like. With the use of the function of the spreadsheet software or the like, correction is performed using regression curves (quadratic regression), parameters for calculating roughness are obtained from data subjected to slope correction, and root-mean-square (RMS) surface roughness is obtained by calculating standard deviation. This surface roughness refers to the surface roughness of part of the particle periphery (at least 400 nm) of the positive electrode active material.

On the surface of the particle of the positive electrode active material 100 of this embodiment, root-mean-square (RMS) surface roughness, which is an index of roughness, is preferably less than 3 nm, further preferably less than 1 nm, still further preferably less than 0.5 nm.

Note that the image processing software used for the noise processing, the interface extraction, or the like is not particularly limited, and for example, “ImageJ” can be used. There is no particular limitation on the spreadsheet software or the like.

For example, the level of surface smoothness of the positive electrode active material 100 can also be quantified from the ratio of an actual specific surface area A_{R} measured by a constant-volume gas adsorption method to an ideal specific surface area Aᵢ.

The ideal specific surface area Aᵢ is calculated on the assumption that all the particles have the same diameter as D50, have the same weight, and have ideal spherical shapes.

The median diameter D50 can be measured with a particle size analyzer or the like using a laser diffraction and scattering method. The specific surface area can be measured with a specific surface area analyzer or the like by a constant-volume gas adsorption method, for example.

In the positive electrode active material 100 of one embodiment of the present invention, the ratio of the actual specific surface area A_{R} to the ideal specific surface area Aᵢ obtained from the median diameter D50 (A_{R}/Aᵢ) is preferably less than or equal to 2.1.

The level of the surface smoothness of the positive electrode active material 100 can be quantified from its cross-sectional SEM image by the following method, for example.

First, a surface SEM image of the positive electrode active material 100 is taken. At this time, conductive coating may be performed as pretreatment for observation. The surface to be observed is preferably vertical to an electron beam. In the case of comparing a plurality of samples, the same measurement conditions and the same observation area are adopted.

Then, the above SEM image is converted into an 8-bit image (which is referred to as a grayscale image) with the use of image processing software (e.g., ImageJ). The grayscale image includes luminance (brightness information). For example, in an 8-bit grayscale image, luminance can be represented by 2⁸=256 gradation levels. A dark portion has a low gradation level and a bright portion has a high gradation level. A variation in luminance can be quantified in relation to the number of gradation levels. The value obtained by the quantification is referred to as a grayscale value. By obtaining such a grayscale value, the unevenness of the positive electrode active material can be evaluated quantitatively.

In addition, a variation in luminance in a target region can also be represented with a histogram. A histogram three-dimensionally shows distribution of gradation levels in a target region and is also referred to as a luminance histogram. A luminance histogram enables visually easy-to-understand evaluation of unevenness of the positive electrode active material.

In the positive electrode active material 100 that can be used for a secondary battery of one embodiment of the present invention, the difference between the maximum grayscale value and the minimum grayscale value is preferably less than or equal to 120, further preferably less than or equal to 115, still further preferably greater than or equal to 70 and less than or equal to 115. The standard deviation of the grayscale value is preferably less than or equal to 11, further preferably less than or equal to 8, still further preferably greater than or equal to 4 and less than or equal to 8.

### <Formation Method of Positive Electrode Active Material>

Next, an example of a method for forming the positive electrode active material 100 that can be used for a secondary battery of one embodiment of the present invention will be described with reference to FIG. 14A to FIG. 14C.

### <Step S11>

In Step S11 shown in FIG. 14A, a lithium source (Li source) and a transition metal M source (M source) are prepared as materials for lithium and a transition metal which are starting materials.

As the lithium source, a lithium-containing compound is preferably used and for example, lithium carbonate, lithium hydroxide, lithium nitrate, lithium fluoride, or the like can be used. The lithium source preferably has a high purity and is preferably a material having a purity of higher than or equal to 99.99%, for example.

The transition metal can be selected from the elements belonging to Groups 4 to 13 of the periodic table and for example, at least one of manganese, cobalt, and nickel is used. As the transition metal, cobalt alone; nickel alone; two metals of cobalt and manganese; two metals of cobalt and nickel; or three metals of cobalt, manganese, and nickel may be used. When cobalt alone is used, the positive electrode active material to be obtained contains lithium cobalt oxide (LCO); when three metals of cobalt, manganese, and nickel are used, the positive electrode active material to be obtained contains lithium nickel cobalt manganese oxide (NCM).

As the transition metal source, a compound containing the above transition metal is preferably used and for example, an oxide, a hydroxide, or the like of any of the metals given as examples of the transition metal can be used. As a cobalt source, cobalt oxide, cobalt hydroxide, or the like can be used. As a manganese source, manganese oxide, manganese hydroxide, or the like can be used. As a nickel source, nickel oxide, nickel hydroxide, or the like can be used.

The transition metal source preferably has a high purity and is preferably a material having a purity of higher than or equal to 3N (99.9%), further preferably higher than or equal to 4N (99.99%), still further preferably higher than or equal to 4N5 (99.995%), yet still further preferably higher than or equal to 5N (99.999%), for example. Impurities of the positive electrode active material can be controlled by using such a high-purity material. As a result, a secondary battery with an increased capacity and/or increased reliability can be obtained.

Furthermore, the transition metal source preferably has high crystallinity and for example, the transition metal source preferably includes single crystal particles. The crystallinity of the transition metal source can be evaluated with a transmission electron microscope (TEM) image, a scanning transmission electron microscope (STEM) image, a high-angle annular dark-field scanning transmission electron microscope (HAADF-STEM) image, or an annular bright-field scanning transmission electron microscope (ABF-STEM) image or by X-ray diffraction (XRD), electron diffraction, neutron diffraction, or the like. Note that the above methods for evaluating crystallinity can also be employed to evaluate the crystallinity of materials other than the transition metal source.

In the case of using two or more transition metal sources, the two or more transition metal sources are preferably prepared to have proportions (mixing ratio) such that a layered rock-salt crystal structure would be obtained.

### <Step S12>

Next, in Step S12 shown in FIG. 14A, the lithium source and the transition metal source are ground and mixed to form a mixed material. The grinding and mixing can be performed by a dry method or a wet method. A wet method is preferred because it can crush a material into a smaller size. When a wet method is employed, a solvent is prepared. As the solvent, ketone such as acetone, alcohol such as ethanol or isopropanol, ether, dioxane, acetonitrile, N-methyl-2-pyrrolidone (NMP), or the like can be used. An aprotic solvent, which is unlikely to react with lithium, is further preferably used. In this embodiment, dehydrated acetone with a purity of higher than or equal to 99.5% is used. It is preferable that the lithium source and the transition metal source be mixed into dehydrated acetone whose moisture content is less than or equal to 10 ppm and which has a purity of higher than or equal to 99.5% in the grinding and mixing. With the use of dehydrated acetone with the above-described purity, impurities that might be mixed can be reduced.

A ball mill, a bead mill, or the like can be used for the grinding and mixing. When a ball mill is used, aluminum balls or zirconium balls are preferably used as a grinding medium. Zirconium balls are preferable because they release fewer impurities. When a ball mill, a bead mill, or the like is used, the peripheral speed is preferably higher than or equal to 100 mm/s and lower than or equal to 2000 mm/s in order to inhibit contamination from the medium. In this embodiment, the grinding and mixing are performed at a peripheral speed of 838 mm/s (the number of rotations: 400 rpm, the ball mill diameter: 40 mm).

### <Step S13>

Next, in Step S13 shown in FIG. 14A, the above mixed material is heated. The heating is preferably performed at higher than or equal to 800° C. and lower than or equal to 1100° C., further preferably at higher than or equal to 900° C. and lower than or equal to 1000° C., still further preferably at approximately 950° C. An excessively low temperature might lead to insufficient decomposition and melting of the lithium source and the transition metal source. An excessively high temperature might lead to a defect due to evaporation of lithium from the lithium source and/or excessive reduction of the metal used as the transition metal source, for example. The defect is, for example, an oxygen defect which could be induced by a change of trivalent cobalt into divalent cobalt due to excessive reduction, in the case where cobalt is used as the transition metal.

The heating time is preferably longer than or equal to 1 hour and shorter than or equal to 100 hours, further preferably longer than or equal to 2 hours and shorter than or equal to 20 hours.

A temperature raising rate is preferably higher than or equal to 80° C./h and lower than or equal to 250° C./h, although depending on the end-point temperature of the heating. For example, in the case of heating at 1000° C. for 10 hours, the temperature raising rate is preferably 200° C./h.

The heating is preferably performed in an atmosphere with little water such as a dry-air atmosphere and for example, the dew point of the atmosphere is preferably lower than or equal to −50° C., further preferably lower than or equal to −80° C. In this embodiment, the heating is performed in an atmosphere with a dew point of −93° C. To reduce impurities that might enter the material, the concentrations of impurities such as CH₄, CO, CO₂, and H₂ in the heating atmosphere are each preferably lower than or equal to 5 ppb (parts per billion).

The heating atmosphere is preferably an oxygen-containing atmosphere. In a method, a dry air is continuously introduced into a reaction chamber. The flow rate of a dry air in this case is preferably 10 L/min. Continuously introducing oxygen into a reaction chamber to make oxygen flow therein is referred to as “flowing”.

In the case where the heating atmosphere is an oxygen-containing atmosphere, flowing is not necessarily performed. For example, the following method may be employed: the pressure in the reaction chamber is reduced, then the reaction chamber is filled with oxygen, and the oxygen is prevented from entering or exiting from the reaction chamber; this method is referred to as purging. For example, the pressure in the reaction chamber may be reduced to −970 hPa and then, the reaction chamber may be filled with oxygen until the pressure becomes 50 hPa.

Cooling after the heating can be performed by letting the mixed material stand to cool, and the time it takes for the temperature to decrease to room temperature from a predetermined temperature is preferably longer than or equal to 10 hours and shorter than or equal to 50 hours. Note that the temperature does not necessarily need to decrease to room temperature as long as it decreases to a temperature acceptable to the next step.

The heating in this step may be performed with a rotary kiln or a roller hearth kiln. Heating with stirring can be performed in either case of a sequential rotary kiln or a batch-type rotary kiln.

A crucible used at the time of the heating is preferably an alumina crucible. An alumina crucible has a material property that hardly releases impurities. In this embodiment, a crucible made of alumina with a purity of 99.9% is used. The heating is preferably performed with the crucible covered with a lid, in which case volatilization of a material can be prevented.

The heated material is ground as needed and may be made to pass through a sieve. Before collection of the heated material, the material may be moved from the crucible to a mortar. As the mortar, an alumina mortar can be suitably used. An alumina mortar has a material property that hardly releases impurities. Specifically, a mortar made of alumina with a purity of higher than or equal to 90%, preferably higher than or equal to 99% is used. Note that heating conditions equivalent to those in Step S13 can be employed in a later-described heating step other than Step S13.

### <Step S14>

Through the above steps, a composite oxide including the transition metal (LiMO₂) can be obtained in Step S14 shown in FIG. 14A. The composite oxide needs to have a crystal structure of a lithium composite oxide represented by LiMO₂, but the composition is not strictly limited to Li:M:O=1:1:2. When the transition metal is cobalt, the composite oxide is referred to as a composite oxide containing cobalt and is represented by LiCoO₂. The composition is not strictly limited to Li:Co:O=1:1:2.

Although the example is described in which the composite oxide is formed by a solid phase method as in Step S11 to Step S14, the composite oxide may be formed by a coprecipitation method. Alternatively, the composite oxide may be formed by a hydrothermal method.

### <Step S15>

Next, in Step S15 shown in FIG. 14A, the above composite oxide is heated. The heating in Step S15 is the first heating performed on the composite oxide and thus, this heating is sometimes referred to as the initial heating. Through the initial heating, the surface of the composite oxide becomes smooth. Having a smooth surface refers to a state where the composite oxide has little unevenness and is rounded as a whole and its corner portion is rounded. A smooth surface also refers to a surface to which few foreign matters are attached. Foreign matters are deemed to cause unevenness and are preferably not attached to a surface.

The initial heating is heating performed after a composite oxide is obtained, and the initial heating for making the surface smooth can sometimes reduce degradation after charge and discharge. The initial heating for making the surface smooth does not need a lithium compound source.

Alternatively, the initial heating for making the surface smooth does not need an added element source.

Alternatively, the initial heating for making the surface smooth does not need a flux.

The initial heating is performed before Step S20 described below and is sometimes referred to as preheating or pretreatment.

The lithium source and transition metal source prepared in Step S11 and the like might contain impurities. The initial heating can reduce impurities in the composite oxide obtained in Step S14.

The heating conditions in this step can be freely set as long as the heating makes the surface of the above composite oxide smooth. For example, any of the heating conditions described for Step S13 can be selected. Additionally, the heating temperature in this step is preferably lower than that in Step S13 so that the crystal structure of the composite oxide is maintained. The heating time in this step is preferably shorter than that in Step S13 so that the crystal structure of the composite oxide is maintained. For example, the heating is preferably performed at a temperature of higher than or equal to 700° C. and lower than or equal to 1000° C. for longer than or equal to 2 hours.

The heating in Step S13 might cause a temperature difference between the surface and an inner portion of the composite oxide. The temperature difference sometimes induces differential shrinkage. It can also be deemed that the temperature difference leads to a fluidity difference between the surface and the inner portion, thereby causing differential shrinkage. The energy involved in differential shrinkage causes a difference in internal stress in the composite oxide. The difference in internal stress is also called distortion, and the above energy is sometimes referred to as distortion energy. The internal stress is eliminated by the initial heating in Step S15 and in other words, the distortion energy is probably equalized by the initial heating in Step S15. When the distortion energy is equalized, the distortion in the composite oxide is relieved. This is probably why the surface of the composite oxide becomes smooth, or “surface improvement is achieved”, through Step S15. In other words, it is deemed that Step S15 reduces the differential shrinkage caused in the composite oxide to make the surface of the composite oxide smooth.

Such differential shrinkage might cause a micro shift in the composite oxide such as a shift in a crystal. To reduce the shift, this step is preferably performed. Performing this step can distribute a shift uniformly in the composite oxide. When the shift is distributed uniformly, the surface of the composite oxide might become smooth, or “crystal grains might be aligned”. In other words, it is deemed that Step S15 reduces the shift in a crystal or the like which is caused in the composite oxide to make the surface of the composite oxide smooth.

In a secondary battery including a composite oxide with a smooth surface as a positive electrode active material, degradation by charge and discharge is suppressed and a crack in the positive electrode active material can be prevented.

It can be said that when surface unevenness information in one cross section of a composite oxide is quantified with measurement data, a smooth surface of the composite oxide has a surface roughness of less than or equal to 10 nm. The one cross section is, for example, a cross section obtained in observation using a scanning transmission electron microscope (STEM).

Note that a pre-synthesized composite oxide containing lithium, a transition metal, and oxygen may be used in Step S14. In this case, Step S11 to Step S13 can be skipped. When Step S15 is performed on the pre-synthesized composite oxide, a composite oxide with a smooth surface can be obtained.

The initial heating might decrease lithium in the composite oxide. An added element described for Step S20 or the like below might easily enter the composite oxide owing to the decrease in lithium.

### <Step S20>

An added element X may be added to the composite oxide having a smooth surface as long as a layered rock-salt crystal structure can be obtained. When the added element X is added to the composite oxide having a smooth surface, the added element can be uniformly added. It is thus preferable that the initial heating precede the addition of the added element. The step of adding the added element is described with reference to FIG. 14B and FIG. 14C.

### <Step S21>

In Step S21 shown in FIG. 14B, an added element source (X source) to be added to the composite oxide is prepared. A lithium source may be prepared in addition to the added element source.

As the added element, one or more elements selected from nickel, cobalt, magnesium, calcium, chlorine, fluorine, aluminum, manganese, titanium, zirconium, yttrium, vanadium, iron, chromium, niobium, lanthanum, hafnium, zinc, silicon, sulfur, phosphorus, boron, and arsenic can be used. As the added element, one or more selected from bromine and beryllium can be used. Note that the added elements given earlier are more suitable since bromine and beryllium are elements having toxicity to living things.

When magnesium is selected as the added element, the added element source can be referred to as a magnesium source. As the magnesium source, magnesium fluoride, magnesium oxide, magnesium hydroxide, magnesium carbonate, or the like can be used. Two or more of these magnesium sources may be used.

When fluorine is selected as the added element, the added element source can be referred to as a fluorine source. As the fluorine source, for example, lithium fluoride (LiF), magnesium fluoride (MgF₂), aluminum fluoride (AlF₃), titanium fluoride (TiF₄), cobalt fluoride (CoF₂ and CoF₃), nickel fluoride (NiF₂), zirconium fluoride (ZrF₄), vanadium fluoride (VF₅), manganese fluoride, iron fluoride, chromium fluoride, niobium fluoride, zinc fluoride (ZnF₂), calcium fluoride (CaF₂), sodium fluoride (NaF), potassium fluoride (KF), barium fluoride (BaF₂), cerium fluoride (CeF₂), lanthanum fluoride (LaF₃), sodium aluminum hexafluoride (Na₃AlF₆), or the like can be used. In particular, lithium fluoride is preferable because it is easily melted in a heating process described later owing to its relatively low melting point of 848° C.

Magnesium fluoride can be used as both the fluorine source and the magnesium source. Lithium fluoride can be used as the lithium source. Another example of the lithium source that can be used in Step S21 is lithium carbonate.

The fluorine source may be a gas; for example, fluorine (F2), carbon fluoride, sulfur fluoride, oxygen fluoride (e.g., OF₂, O₂F₂, O₃F₂, O₄F₂, and O₂F), or the like may be used and mixed in the atmosphere in a heating step described later. Two or more of these fluorine sources may be used.

In this embodiment, lithium fluoride (LiF) is prepared as the fluorine source, and magnesium fluoride (MgF₂) is prepared as the fluorine source and the magnesium source. When lithium fluoride and magnesium fluoride are mixed at a molar ratio of approximately LiF:MgF₂=65:35, the effect of lowering the melting point is maximized. Meanwhile, when the proportion of lithium fluoride increases, the cycle performance might deteriorate because of an excessive amount of lithium. Therefore, the molar ratio of lithium fluoride to magnesium fluoride is preferably LiF:MgF₂=x:1 (0≤x≤1.9), further preferably LiF:MgF₂=x:1 (0.1≤x≤0.5), still further preferably LiF:MgF₂=x:1 (x=0.33 or an approximate value thereof). Note that in this specification and the like, the expression “an approximate value of a given value” means greater than 0.9 times and smaller than 1.1 times the given value.

Meanwhile, magnesium is preferably added at greater than 0.1 at % and less than or equal to 3 at %, further preferably greater than or equal to 0.5 at % and less than or equal to 2 at %, still further preferably greater than or equal to 0.5 at % and less than or equal to 1 at %, relative to LiMO₂. When magnesium is added at less than or equal to 0.1 at %, the initial discharge capacity is high but repeated high voltage charge and discharge rapidly lowers the discharge capacity. In the case where magnesium is added at greater than 0.1 at % and less than or equal to 3 at %, both the initial discharge characteristics and charge and discharge cycle performance are excellent even when high voltage charge and discharge are repeated. By contrast, in the case where magnesium is added at greater than 3 at %, both the initial discharge capacity and the charge and discharge cycle performance tend to gradually degrade.

### <Step S22>

Next, in Step S22 shown in FIG. 14B, the magnesium source and the fluorine source are ground and mixed. Any of the conditions for the grinding and mixing that are described for Step S12 can be selected to perform this step.

A heating step may be performed after Step S22 as needed. For the heating step, any of the heating conditions described for Step S13 can be selected. The heating time is preferably longer than or equal to 2 hours and the heating temperature is preferably higher than or equal to 800° C. and lower than or equal to 1100° C.

### <Step S23>

Next, in Step S23 shown in FIG. 14B, the materials ground and mixed in the above step are collected to give the added element source (X source). Note that the added element source in Step S23 contains a plurality of starting materials and can be referred to as a mixture.

As for the particle diameter of the mixture, its D50 (median diameter) is preferably greater than or equal to 10 nm and less than or equal to 20 μm, further preferably greater than or equal to 100 nm and less than or equal to 5 μm. Also when one kind of material is used as the added element source, the D50 (median diameter) is preferably greater than or equal to 10 nm and less than or equal to 20 μm, further preferably greater than or equal to 100 nm and less than or equal to 5 μm.

Such a pulverized mixture (which may contain only one kind of the added element) is easily attached to the surface of a composite oxide particle uniformly in a later step of mixing with the composite oxide. The mixture is preferably attached uniformly to the surface of the composite oxide particle, in which case fluorine and magnesium are easily distributed or dispersed uniformly in a surface portion of the composite oxide after heating. The region where fluorine and magnesium are distributed can be referred to as a surface portion. When there is a region containing neither fluorine nor magnesium in the surface portion, an O3′ type crystal structure, which is described later, might be unlikely to be obtained in a charged state. Note that although fluorine is used in the above description, chlorine may be used instead of fluorine, and a general term “halogen” for these elements can replace “fluorine”.

### <Step S21>

A process different from that in FIG. 14B is described with reference to FIG. 14C. In Step S21 shown in FIG. 14C, four kinds of added element sources to be added to the composite oxide are prepared. In other words, FIG. 14C is different from FIG. 14B in the kinds of the added element sources. A lithium source may be prepared together with the added element sources.

As the four kinds of added element sources, a magnesium source (Mg source), a fluorine source (F source), a nickel source (Ni source), and an aluminum source (Al source) are prepared. Note that the magnesium source and the fluorine source can be selected from the compounds and the like described with reference to FIG. 14B. As the nickel source, nickel oxide, nickel hydroxide, or the like can be used. As the aluminum source, aluminum oxide, aluminum hydroxide, or the like can be used.

### <Step S22 and Step S23>

Step S22 and Step S23 shown in FIG. 14C are similar to the steps described with reference to FIG. 14B.

### <Step S31>

Next, in Step S31 shown in FIG. 14A, the composite oxide and the added element source (X source) are mixed. The atomic ratio of the transition metal M in the composite oxide containing lithium, the transition metal, and oxygen to magnesium Mg contained in the added element X is preferably M:Mg=100:y (0.1≤y≤6), further preferably M:Mg=100:y (0.3≤y≤3).

The mixing in Step S31 is preferably performed under milder conditions than the mixing in Step S12, in order not to damage the composite oxide particles. For example, a condition with a smaller number of rotations or a shorter time than that for the mixing in Step S12 is preferable. Moreover, a dry method is regarded as a milder condition than a wet method. For example, a ball mill or a bead mill can be used for the mixing. When a ball mill is used, zirconium balls are preferably used as a medium, for example.

In this embodiment, the mixing is performed with a ball mill using zirconium balls with a diameter of 1 mm by a dry method at 150 rpm for 1 hour. The mixing is performed in a dry room the dew point of which is higher than or equal to −100° C. and lower than or equal to −10° C.

### <Step S32>

Next, in Step S32 in FIG. 14A, the materials mixed in the above step are collected, whereby a mixture 903 is obtained. At the time of the collection, the materials may be crushed as needed and made to pass through a sieve.

Note that in this embodiment, the method is described in which lithium fluoride as the fluorine source and magnesium fluoride as the magnesium source are added afterward to the composite oxide that has been subjected to the initial heating. However, the present invention is not limited to the above method. The magnesium source, the fluorine source, and the like can be added to the lithium source and the transition metal source in Step S11, i.e., at the stage of the starting materials of the composite oxide. Then, the heating in Step S13 is performed, so that LiMO₂ to which magnesium and fluorine are added can be obtained. In that case, there is no need to separately perform Step S11 to Step S14 and Step S21 to Step S23. This method can be regarded as being simple and highly productive.

Alternatively, lithium cobalt oxide to which magnesium and fluorine are added in advance may be used. When lithium cobalt oxide to which magnesium and fluorine are added is used, Step S11 to Step S32 and Step S20 can be skipped. This method can be regarded as being simple and highly productive.

Alternatively, to lithium cobalt oxide to which magnesium and fluorine are added in advance, a magnesium source and a fluorine source, or a magnesium source, a fluorine source, a nickel source, and an aluminum source may be further added as in Step S20.

### <Step S33>

Then, in Step S33 shown in FIG. 14A, the mixture 903 is heated. Any of the heating conditions described for Step S13 can be selected. The heating time is preferably longer than or equal to 2 hours.

Here, a supplementary explanation of the heating temperature is provided. The lower limit of the heating temperature in Step S33 needs to be higher than or equal to the temperature at which a reaction between the composite oxide (LiMO₂) and the added element source proceeds. The temperature at which the reaction proceeds is the temperature at which interdiffusion of the elements included in LiMO₂ and the added element source occurs, and may be lower than the melting temperatures of these materials. It is known that in the case of an oxide as an example, solid phase diffusion occurs at the Tamman temperature T_{d} (0.757 times the melting temperature Tₘ). Accordingly, it is only required that the heating temperature in Step S33 be higher than or equal to 500° C.

Needless to say, the reaction more easily proceeds at a temperature higher than or equal to the temperature at which at least part of the mixture 903 is melted. For example, in the case where LiF and MgF₂ are included in the added element source, the lower limit of the heating temperature in Step S33 is preferably higher than or equal to 742° C. because the eutectic point of LiF and MgF₂ is around 742° C.

The mixture 903 obtained by mixing such that LiCoO₂:LiF:MgF₂=100:0.33:1 (molar ratio) exhibits an endothermic peak at around 830° C. in differential scanning calorimetry (DSC) measurement. Therefore, the lower limit of the heating temperature is further preferably higher than or equal to 830° C.

A higher heating temperature is preferable because it facilitates the reaction, shortens the heating time, and enables high productivity.

The upper limit of the heating temperature is lower than the decomposition temperature of LiMO₂ (the decomposition temperature of LiCoO₂ is 1130° C.). At around the decomposition temperature, a slight amount of LiMO₂ might be decomposed. Thus, the upper limit of the heating temperature is preferably lower than or equal to 1000° C., further preferably lower than or equal to 950° C., still further preferably lower than or equal to 900° C.

In view of the above, the heating temperature in Step S33 is preferably higher than or equal to 500° C. and lower than or equal to 1130° C., further preferably higher than or equal to 500° C. and lower than or equal to 1000° C., still further preferably higher than or equal to 500° C. and lower than or equal to 950° C., yet still further preferably higher than or equal to 500° C. and lower than or equal to 900° C. Furthermore, the heating temperature in Step S33 is preferably higher than or equal to 742° C. and lower than or equal to 1130° C., further preferably higher than or equal to 742° C. and lower than or equal to 1000° C., still further preferably higher than or equal to 742° C. and lower than or equal to 950° C., yet still further preferably higher than or equal to 742° C. and lower than or equal to 900° C. Furthermore, the heating temperature in Step S33 is preferably higher than or equal to 800° C. and lower than or equal to 1100° C., further preferably higher than or equal to 830° C. and lower than or equal to 1130° C., still further preferably higher than or equal to 830° C. and lower than or equal to 1000° C., yet still further preferably higher than or equal to 830° C. and lower than or equal to 950° C., yet still further preferably higher than or equal to 830° C. and lower than or equal to 900° C. Note that the heating temperature in Step S33 is preferably higher than that in Step S13.

In addition, at the time of heating the mixture 903, the partial pressure of fluorine or a fluoride originating from the fluorine source or the like is preferably controlled to be within an appropriate range.

In the formation method described in this embodiment, some of the materials, e.g., LiF as the fluorine source, function as a fusing agent in some cases. Owing to this function, the heating temperature can be lower than the decomposition temperature of the composite oxide (LiMO₂), e.g., higher than or equal to 742° C. and lower than or equal to 950° C., which allows distribution of the added element such as magnesium in the surface portion and formation of a positive electrode active material having favorable characteristics.

However, since LiF in a gas phase has a specific gravity less than that of oxygen, heating might volatilize LiF and in that case, LiF in the mixture 903 decreases. As a result, the function of a fusing agent deteriorates. Therefore, heating needs to be performed while volatilization of LiF is inhibited. Note that even when LiF is not used as the fluorine source or the like, Li at the surface of LiMO₂ and F of the fluorine source might react to produce LiF, which might be volatilized. Therefore, such inhibition of volatilization is needed also when a fluoride having a higher melting point than LiF is used.

In view of this, the mixture 903 is preferably heated in an atmosphere containing LiF, i.e., the mixture 903 is preferably heated in a state where the partial pressure of LiF in a heating furnace is high. Such heating can inhibit volatilization of LiF in the mixture 903.

The heating in this step is preferably performed such that the particles of the mixture 903 are not adhered to each other. Adhesion of the particles of the mixture 903 during the heating might decrease the area of contact with oxygen in the atmosphere and inhibit a path of diffusion of the added element (e.g., fluorine), thereby hindering distribution of the added element (e.g., magnesium and fluorine) in the surface portion.

It is considered that uniform distribution of the added element (e.g., fluorine) in the surface portion leads to a smooth positive electrode active material with little unevenness. Thus, it is preferable that the particles not be adhered to each other in order to allow the smooth surface obtained through the heating in Step S15 to be maintained or to be smoother in this step.

In the case of using a rotary kiln for the heating, the flow rate of an oxygen-containing atmosphere in the kiln is preferably controlled during the heating. For example, the flow rate of an oxygen-containing atmosphere is preferably set low, or no flowing of an atmosphere is preferably performed after an atmosphere is purged first and an oxygen atmosphere is introduced into the kiln. Flowing of oxygen is not preferable because it might cause evaporation of the fluorine source, which prevents maintaining the smoothness of the surface.

In the case of using a roller hearth kiln for the heating, the mixture 903 can be heated in an atmosphere containing LiF with the container in which the mixture 903 is put covered with a lid.

A supplementary explanation of the heating time is provided. The heating time depends on conditions such as the heating temperature and the particle size and composition of LiMO₂ in Step S14. The heating may be preferably performed at a lower temperature or for a shorter time in the case where the particle size is small than in the case where the particle size is large.

In the case where the composite oxide (LiMO₂) in Step S14 in FIG. 14A has a median diameter (D50) of approximately 12 μm, the heating temperature is preferably higher than or equal to 600° C. and lower than or equal to 950° C., for example. The heating time is preferably longer than or equal to 3 hours, further preferably longer than or equal to 10 hours, still further preferably longer than or equal to 60 hours, for example. Note that the time for lowering the temperature after the heating is preferably longer than or equal to 10 hours and shorter than or equal to 50 hours, for example.

In the case where the composite oxide (LiMO₂) in Step S14 has a median diameter (D50) of approximately 5 μm, the heating temperature is preferably higher than or equal to 600° C. and lower than or equal to 950° C., for example. The heating time is preferably longer than or equal to 1 hour and shorter than or equal to 10 hours, further preferably approximately 2 hours, for example. Note that the time for lowering the temperature after the heating is preferably longer than or equal to 10 hours and shorter than or equal to 50 hours, for example.

### <Step S34>

Next, the heated material is collected in Step S34 shown in FIG. 14A, in which crushing is performed as needed; thus, the positive electrode active material 100 is obtained. Here, the collected particles are preferably made to pass through a sieve. Through the above process, the positive electrode active material 100 of one embodiment of the present invention can be formed. The positive electrode active material of one embodiment of the present invention has a smooth surface.

This embodiment can be used in combination with any of the other embodiments.

### Embodiment 3

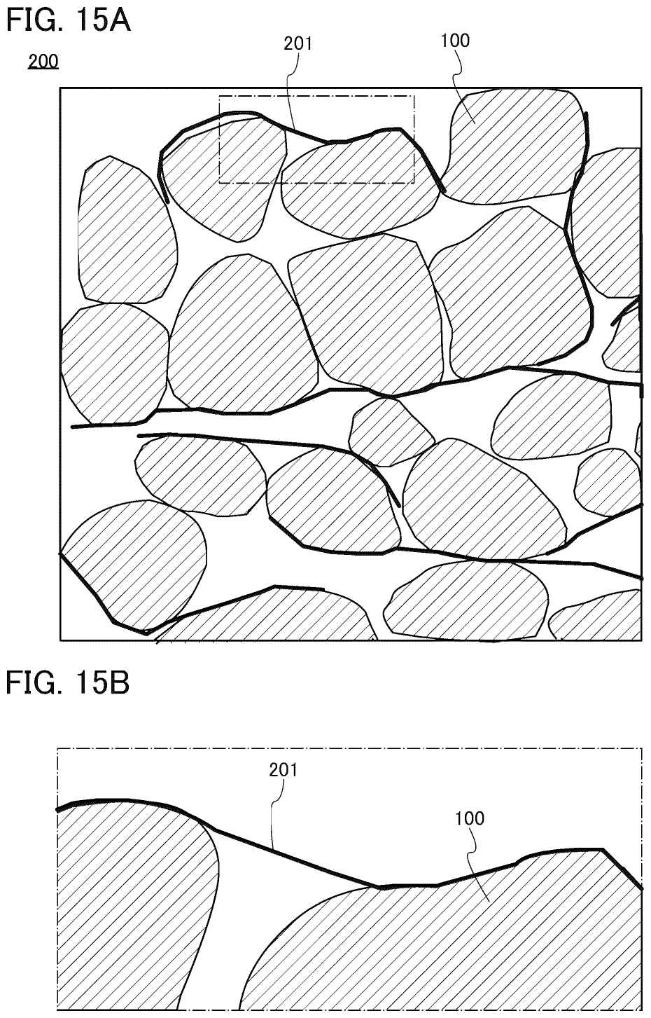

In this embodiment, an electrolyte, a material other than a positive electrode active material, and a structure that can be used for a secondary battery of one embodiment of the present invention are described with reference to FIG. 15A and FIG. 15B.

### [Positive Electrode]

A positive electrode includes a positive electrode active material layer and a positive electrode current collector. The positive electrode active material layer includes a positive electrode active material, and may include a conductive material and a binder. As the positive electrode active material, the positive electrode active material described in the above embodiment can be used.

### [Conductive Material]

Examples of the conductive material include acetylene black (AB), graphite (black lead) particles, carbon nanotubes, graphene, and a graphene compound.

A cross-sectional structure example of an active material layer 200 containing graphene or a graphene compound as a conductive material is described below.

FIG. 15A is a longitudinal cross-sectional view of the active material layer 200. The active material layer 200 includes particles of the positive electrode active material 100, graphene or a graphene compound 201 serving as the conductive material, and a binder (not illustrated).

The graphene compound 201 in this specification and the like refers to multilayer graphene, multi graphene, graphene oxide, multilayer graphene oxide, multi graphene oxide, reduced graphene oxide, reduced multilayer graphene oxide, reduced multi graphene oxide, graphene quantum dots, and the like. A graphene compound contains carbon, has a plate-like shape, a sheet-like shape, or the like, and has a two-dimensional structure formed of a six-membered ring composed of carbon atoms. The two-dimensional structure formed of the six-membered ring composed of carbon atoms may be referred to as a carbon sheet. A graphene compound may include a functional group. The graphene compound is preferably bent. The graphene compound may be rounded like a carbon nanofiber.

In this specification and the like, graphene oxide contains carbon and oxygen, has a sheet-like shape, and includes a functional group, in particular, an epoxy group, a carboxy group, or a hydroxy group.

In this specification and the like, reduced graphene oxide contains carbon and oxygen, has a sheet-like shape, and has a two-dimensional structure formed of a six-membered ring composed of carbon atoms. The reduced graphene oxide may also be referred to as a carbon sheet. The reduced graphene oxide functions by itself and may have a stacked-layer structure. The reduced graphene oxide preferably includes a portion where the carbon concentration is higher than 80 atomic % and the oxygen concentration is higher than or equal to 2 atomic % and lower than or equal to 15 atomic %. With such a carbon concentration and such an oxygen concentration, the reduced graphene oxide can function as a conductive material with high conductivity even with a small amount. In addition, the intensity ratio G/D of a G band to a D band of the Raman spectrum of the reduced graphene oxide is preferably 1 or more. The reduced graphene oxide with such an intensity ratio can function as a conductive material with high conductivity even with a small amount.

A graphene compound sometimes has excellent electrical characteristics of high conductivity and excellent physical properties of high flexibility and high mechanical strength. A graphene compound has a sheet-like shape. A graphene compound has a curved surface in some cases, thereby enabling low-resistant surface contact. Furthermore, a graphene compound sometimes has extremely high conductivity even with a small thickness, and thus a small amount of a graphene compound efficiently allows a conductive path to be formed in an active material layer. Hence, a graphene compound is preferably used as the conductive material, in which case the area where the active material and the conductive material are in contact with each other can be increased. The graphene compound preferably covers 80% or more of the active material. Note that the graphene compound preferably clings to at least part of an active material particle. The graphene compound preferably overlays at least part of the active material particle. The shape of the graphene compound preferably conforms to at least part of the shape of the active material particle. The shape of an active material particle means, for example, unevenness of a single active material particle or unevenness formed by a plurality of active material particles. The graphene compound preferably surrounds at least part of an active material particle. The graphene compound may have a hole.

In the case where active material particles with a small diameter (e.g., 1 μm or less) are used, the specific surface area of the active material particles is large and thus more conductive paths for the active material particles are needed. In such a case, it is particularly preferable to use a graphene compound that can efficiently form a conductive path even with a small amount.

It is particularly effective to use a graphene compound, which has the above-described properties, as a conductive material of a secondary battery that needs to be rapidly charged and discharged. For example, a secondary battery for a two- or four-wheeled vehicle, a secondary battery for a drone, or the like is required to have fast charge and fast discharge characteristics in some cases. In addition, a mobile electronic device or the like is required to have fast charge characteristics in some cases. Fast charge and fast discharge may also be referred to as charge at a high rate and discharge at a high rate, for example, at 1 C, 2 C, or 5 C or more.

The longitudinal cross section of the active material layer 200 in FIG. 15B shows substantially uniform dispersion of the sheet-like graphene or the graphene compound 201 in the active material layer 200. The graphene or the graphene compound 201 is schematically shown by the thick line in FIG. 15B but is actually a thin film having a thickness corresponding to the thickness of a single layer or a multi-layer of carbon molecules. A plurality of sheets of graphene or the plurality of graphene compounds 201 are formed to partly cover or adhere to the surfaces of the plurality of particles of the positive electrode active material 100, so that the plurality of sheets of graphene or the plurality of graphene compounds 201 make surface contact with the particles of the positive electrode active material 100.

Here, the plurality of sheets of graphene or the plurality of graphene compounds can be bonded to each other to form a net-like graphene compound sheet (hereinafter, referred to as a graphene compound net or a graphene net). A graphene net that covers the active material can function as a binder for bonding the active material particles. Accordingly, the amount of the binder can be reduced, or the binder does not have to be used. This can increase the proportion of the active material in the electrode volume and/or the electrode weight. That is to say, the charge and discharge capacity of the secondary battery can be increased.

Here, it is preferable to perform reduction after a layer to be the active material layer 200 is formed in such a manner that graphene oxide is used as the graphene or the graphene compound 201 and mixed with an active material. That is, the formed active material layer preferably contains reduced graphene oxide. When graphene oxide with extremely high dispersibility in a polar solvent is used for the formation of the graphene or the graphene compound 201, the graphene or the graphene compound 201 can be substantially uniformly dispersed in the active material layer 200. The solvent is removed by volatilization from a dispersion medium in which graphene oxide is uniformly dispersed, and the graphene oxide is reduced; hence, the sheets of graphene or the graphene compounds 201 remaining in the active material layer 200 partly overlap with each other and are dispersed such that surface contact is made, thereby forming a three-dimensional conduction path. Note that graphene oxide can be reduced by heat treatment or with the use of a reducing agent, for example.

Unlike a conductive material in the form of particles, such as acetylene black, which makes point contact with an active material, the graphene or the graphene compound 201 is capable of making low-resistance surface contact; accordingly, the electrical conduction between the particles of the positive electrode active material 100 and the graphene or the graphene compound 201 can be improved with a small amount of the graphene and the graphene compound 201 compared with a normal conductive material. Thus, the proportion of the positive electrode active material 100 in the active material layer 200 can be increased, resulting in increased discharge capacity of the secondary battery.

It is possible to form, with a spray dry apparatus, a graphene compound serving as a conductive material as a coating film to cover the entire surface of the active material in advance and to form a conductive path between the active materials using the graphene compound.

A material used in formation of the graphene compound may be mixed with the graphene compound to be used for the active material layer 200. For example, particles used as a catalyst in formation of the graphene compound may be mixed with the graphene compound. As an example of the catalyst in formation of the graphene compound, particles containing any of silicon oxide (SiO₂ or SiOₓ (x<2)), aluminum oxide, iron, nickel, ruthenium, iridium, platinum, copper, germanium, and the like can be given. The median diameter (D50) of the particles is preferably less than or equal to 1 μm, further preferably less than or equal to 100 nm.

### [Binder]

As the binder, a rubber material such as styrene-butadiene rubber (SBR), styrene-isoprene-styrene rubber, acrylonitrile-butadiene rubber, butadiene rubber, or ethylene-propylene-diene copolymer is preferably used, for example. Fluororubber can also be used as the binder.

As the binder, for example, water-soluble polymers are preferably used. As the water-soluble polymers, a polysaccharide can be used, for example. As the polysaccharide, starch, a cellulose derivative such as carboxymethyl cellulose (CMC), methyl cellulose, ethyl cellulose, hydroxypropyl cellulose, diacetyl cellulose, or regenerated cellulose, or the like can be used. It is further preferable that such water-soluble polymers be used in combination with any of the above rubber materials.

Alternatively, as the binder, a material such as polystyrene, poly(methyl acrylate), poly(methyl methacrylate) (PMMA), sodium polyacrylate, polyvinyl alcohol (PVA), polyethylene oxide (PEO), polypropylene oxide, polyimide, polyvinyl chloride, polytetrafluoroethylene, polyethylene, polypropylene, polyisobutylene, polyethylene terephthalate, nylon, polyvinylidene fluoride (PVDF), polyacrylonitrile (PAN), ethylene-propylene-diene polymer, polyvinyl acetate, or nitrocellulose is preferably used.

Two or more of the above materials may be used in combination for the binder.

For example, a material having a significant viscosity modifying effect and another material may be used in combination. For example, a rubber material or the like has high adhesion or high elasticity but may have difficulty in viscosity modification when mixed in a solvent. In such a case, a rubber material or the like is preferably mixed with a material having a significant viscosity modifying effect, for example. As a material having a significant viscosity modifying effect, for instance, a water-soluble polymer is preferably used. As a water-soluble polymer having a significant viscosity modifying effect, the above-mentioned polysaccharide, for instance, a cellulose derivative such as carboxymethyl cellulose (CMC), methyl cellulose, ethyl cellulose, hydroxypropyl cellulose, diacetyl cellulose, or regenerated cellulose or starch can be used.

Note that a cellulose derivative such as carboxymethyl cellulose obtains a higher solubility when converted into a salt such as a sodium salt or an ammonium salt of carboxymethyl cellulose, and thus easily exerts an effect as a viscosity modifier. A high solubility can also increase the dispersibility of an active material and other components in the formation of a slurry for an electrode. In this specification, cellulose and a cellulose derivative used as a binder of an electrode include salts thereof.

A water-soluble polymer stabilizes the viscosity by being dissolved in water and allows stable dispersion of the active material and another material combined as a binder, such as styrene-butadiene rubber, in an aqueous solution. Furthermore, a water-soluble polymer is expected to be easily and stably adsorbed onto an active material surface because it has a functional group. Many cellulose derivatives, such as carboxymethyl cellulose, have a functional group such as a hydroxyl group or a carboxyl group. Because of functional groups, polymers are expected to interact with each other and cover an active material surface in a large area.

In the case where the binder that covers or is in contact with the active material surface forms a film, the film is expected to serve also as a passivation film to suppress the decomposition of the electrolyte. Here, a passivation film refers to a film without electric conductivity or a film with extremely low electric conductivity, and can inhibit the decomposition of an electrolyte at a potential at which a battery reaction occurs when the passivation film is formed on the active material surface, for example. It is preferable that the passivation film can conduct lithium ions while suppressing electrical conduction.

### [Positive Electrode Current Collector]

The positive electrode current collector can be formed using a material that has high conductivity, such as a metal like stainless steel, gold, platinum, aluminum, or titanium, or an alloy thereof. It is preferable that a material used for the positive electrode current collector not be eluted at the potential of the positive electrode. It is also possible to use an aluminum alloy to which an element that improves heat resistance, such as silicon, titanium, neodymium, scandium, or molybdenum, is added. A metal element that forms silicide by reacting with silicon may be used. Examples of the metal element that forms silicide by reacting with silicon include zirconium, titanium, hafnium, vanadium, niobium, tantalum, chromium, molybdenum, tungsten, cobalt, and nickel. The positive electrode current collector can have a foil-like shape, a plate-like shape, a sheet-like shape, a net-like shape, a punching-metal shape, an expanded-metal shape, or the like as appropriate. The positive electrode current collector preferably has a thickness greater than or equal to 5 μm and less than or equal to 30 μm.

### [Negative Electrode]

The negative electrode includes a negative electrode active material layer and a negative electrode current collector. The negative electrode active material layer may contain a conductive material and a binder.

### [Negative Electrode Active Material]

As a negative electrode active material, for example, an alloy-based material or a carbon-based material can be used.

For the negative electrode active material, an element that enables charge and discharge reactions by an alloying reaction and a dealloying reaction with lithium can be used. For example, a material containing at least one of silicon, tin, gallium, aluminum, germanium, lead, antimony, bismuth, silver, zinc, cadmium, indium, and the like can be used. Such elements have higher charge and discharge capacity than carbon; in particular, silicon has a high theoretical capacity of 4200 mAh/g. For this reason, silicon is preferably used as the negative electrode active material. Alternatively, a compound containing any of the above elements may be used. Examples of the compound include SiO, Mg₂Si, Mg₂Ge, SnO, SnO₂, Mg₂Sn, SnS₂, V₂Sn₃, FeSn₂, CoSn₂, Ni₃Sn₂, Cu₆Sn₅, Ag₃Sn, Ag₃Sb, Ni₂MnSb, CeSb₃, LaSn₃, La₃Co₂Sn₇, CoSb₃, InSb, and SbSn. Here, an element that enables charge and discharge reactions by an alloying reaction and a dealloying reaction with lithium, a compound containing the element, and the like may be referred to as an alloy-based material.

In this specification and the like, SiO refers, for example, to silicon monoxide. Note that SiO can alternatively be expressed as SiOₓ. Here, x preferably has an approximate value of 1. For example, x is preferably greater than or equal to 0.2 and less than or equal to 1.5, further preferably greater than or equal to 0.3 and less than or equal to 1.2. Alternatively, x is preferably greater than or equal to 0.2 and less than or equal to 1.2. Still alternatively, x is preferably greater than or equal to 0.3 and less than or equal to 1.5.

Silicon nanoparticles can be used as the negative electrode active material. The average diameter of silicon nanoparticles is preferably greater than or equal to 5 nm and less than 1 μm, further preferably greater than or equal to 10 nm and less than or equal to 300 nm, still further preferably greater than or equal to 10 nm and less than or equal to 100 nm.

The silicon nanoparticles may have crystallinity. The silicon particles may include a region with crystallinity and an amorphous region.

As the negative electrode active material, particles including lithium silicate may be used. The particles including lithium silicate may contain zirconium, yttrium, iron, and the like. The particles including lithium silicate may have a mode in which a single particle includes a plurality of silicon crystal grains.

The average diameter of the particles containing lithium silicate is preferably greater than or equal to 100 nm and less than or equal to 100 μm, further preferably greater than or equal to 500 nm and less than or equal to 50 μm.

As the carbon-based material, graphite, graphitizing carbon (soft carbon), non-graphitizing carbon (hard carbon), carbon nanotube, graphene, carbon black, or the like can be used.

Examples of graphite include artificial graphite and natural graphite. Examples of artificial graphite include mesocarbon microbeads (MCMB), coke-based artificial graphite, and pitch-based artificial graphite. As artificial graphite, spherical graphite having a spherical shape can be used. For example, MCMB is preferably used because it may have a spherical shape. Moreover, MCMB may preferably be used because it can relatively easily have a small surface area. Examples of natural graphite include flake graphite and spherical natural graphite.

Graphite has a low potential substantially equal to that of a lithium metal (greater than or equal to 0.05 V and less than or equal to 0.3 V vs. Li/Li⁺) when lithium ions are inserted into graphite (while a lithium-graphite intercalation compound is formed). For this reason, a lithium-ion secondary battery can have a high operating voltage. In addition, graphite is preferred because of its advantages such as a relatively high charge and discharge capacity per unit volume, relatively small volume expansion, low cost, and a higher level of safety than that of a lithium metal.

As the negative electrode active material, an oxide such as titanium dioxide (TiO₂), lithium titanium oxide (Li₄Ti₅O₁₂), a lithium-graphite intercalation compound (LiₓC₆), niobium pentoxide (Nb₂O₅), tungsten oxide (WO₂), or molybdenum oxide (MoO₂) can be used.

Alternatively, as the negative electrode active material, Li₃₋ₓMₓN (M is Co, Ni, or Cu) with a Li₃N structure, which is a composite nitride of lithium and a transition metal, can be used. For example, Li_{2.6}Co_{0.4}N₃ is preferable because of high charge and discharge capacity (900 mAh/g and 1890 mAh/cm³).

A composite nitride containing lithium and a transition metal is preferably used, in which case lithium ions are contained in the negative electrode active material and thus the negative electrode active material can be used in combination with a material for a positive electrode active material that does not contain lithium ions, such as V₂O₅ or Cr₃O₈. Note that in the case of using a material containing lithium ions as a positive electrode active material, the composite nitride containing lithium and a transition metal can be used as the negative electrode active material by extracting the lithium ions contained in the positive electrode active material in advance.

Alternatively, a material that causes a conversion reaction can be used for the negative electrode active material. For example, a transition metal oxide that does not form an alloy with lithium, such as cobalt oxide (CoO), nickel oxide (NiO), or iron oxide (FeO), may be used as the negative electrode active material. Other examples of the material that causes a conversion reaction include oxides such as Fe₂O₃, CuO, Cu₂O, RuO₂, and Cr₂O₃, sulfides such as CoS_{0.89}, NiS, and CuS, nitrides such as Zn₃N₂, Cu₃N, and Ge₃N₄, phosphides such as NiP₂, FeP₂, and CoP₃, and fluorides such as FeF₃ and BiF₃.

For the conductive material and the binder that can be included in the negative electrode active material layer, materials similar to those of the conductive material and the binder that can be included in the positive electrode active material layer can be used.

### [Negative Electrode Current Collector]

For the negative electrode current collector, a material similar to that of the positive electrode current collector can be used. Note that a material that is not alloyed with carrier ions of lithium or the like is preferably used for the negative electrode current collector.

### [Separator]

The secondary battery preferably includes a separator. The separator can be formed using, for example, paper, nonwoven fabric, glass fiber, ceramics, or synthetic fiber containing nylon (polyamide), vinylon (polyvinyl alcohol-based fiber), polyester, acrylic, polyolefin, or polyurethane. The separator is preferably formed to have an envelope-like shape to wrap one of the positive electrode and the negative electrode.

The separator may have a multilayer structure. For example, an organic material film of polypropylene, polyethylene, or the like can be coated with a ceramic-based material, a fluorine-based material, a polyamide-based material, a mixture thereof, or the like. Examples of the ceramic-based material include aluminum oxide particles and silicon oxide particles. Examples of the fluorine-based material include PVDF and polytetrafluoroethylene. Examples of the polyamide-based material include nylon and aramid (meta-based aramid and para-based aramid).

When the separator is coated with the ceramic-based material, the oxidation resistance is improved; hence, deterioration of the separator in high voltage charge and discharge can be suppressed and thus the reliability of the secondary battery can be improved. When the separator is coated with the fluorine-based material, the separator is easily brought into close contact with an electrode, resulting in high output characteristics. When the separator is coated with the polyamide-based material, in particular, aramid, the safety of the secondary battery is improved because heat resistance is improved.

For example, both surfaces of a polypropylene film may be coated with a mixed material of aluminum oxide and aramid. Alternatively, a surface of a polypropylene film that is in contact with the positive electrode may be coated with a mixed material of aluminum oxide and aramid, and a surface of the polypropylene film that is in contact with the negative electrode may be coated with the fluorine-based material.

With the use of a separator having a multilayer structure, the charge and discharge capacity per volume of the secondary battery can be increased because the safety of the secondary battery can be maintained even when the total thickness of the separator is small.

### [Exterior Body]

For an exterior body included in the secondary battery, a metal material such as aluminum or a resin material can be used, for example. A film-like exterior body can also be used. As the film, for example, it is possible to use a film having a three-layer structure in which a highly flexible metal thin film of aluminum, stainless steel, copper, nickel, or the like is provided over a film formed of a material such as polyethylene, polypropylene, polycarbonate, ionomer, or polyamide, and an insulating synthetic resin film of a polyamide-based resin, a polyester-based resin, or the like is provided over the metal thin film as the outer surface of the exterior body.

This embodiment can be used in appropriate combination with the other embodiments.

### Embodiment 4

In this embodiment, examples of the shape of a secondary battery including the ionic liquid described in the above embodiment are described. For the materials used for the secondary battery described in this embodiment, the description of the above embodiment can be referred to.

### <Coin-Type Secondary Battery>

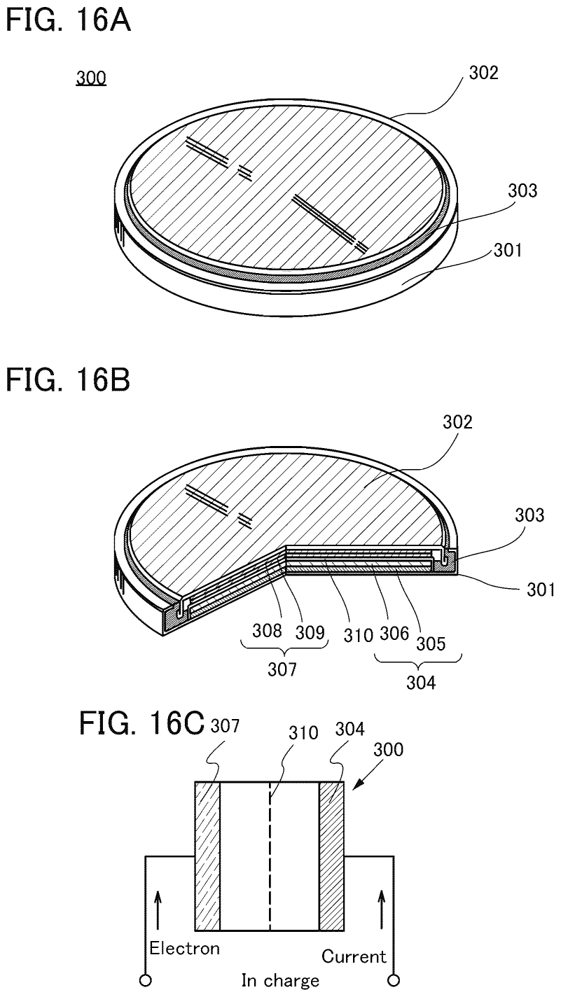

First, an example of a coin-type secondary battery is described. FIG. 16A is an external view of a coin-type (single-layer flat type) secondary battery, and FIG. 16B is a cross-sectional view thereof.

In a coin-type secondary battery 300, a positive electrode can 301 doubling as a positive electrode terminal and a negative electrode can 302 doubling as a negative electrode terminal are insulated from each other and sealed by a gasket 303 made of polypropylene or the like. A positive electrode 304 includes a positive electrode current collector 305 and a positive electrode active material layer 306 provided in contact with the positive electrode current collector 305. A negative electrode 307 includes a negative electrode current collector 308 and a negative electrode active material layer 309 provided in contact with the negative electrode current collector 308.

Note that only one surface of each of the positive electrode 304 and the negative electrode 307 used for the coin-type secondary battery 300 is provided with an active material layer.

For the positive electrode can 301 and the negative electrode can 302, a metal having corrosion resistance to an electrolyte, such as nickel, aluminum, or titanium, an alloy of such a metal, or an alloy of such a metal and another metal (e.g., stainless steel) can be used. Alternatively, the positive electrode can 301 and the negative electrode can 302 are preferably covered with nickel, aluminum, and/or the like in order to prevent corrosion due to the electrolyte. The positive electrode can 301 and the negative electrode can 302 are electrically connected to the positive electrode 304 and the negative electrode 307, respectively.

The negative electrode 307, the positive electrode 304, and a separator 310 are soaked in the electrolyte. Then, as illustrated in FIG. 16B, the positive electrode 304, the separator 310, the negative electrode 307, and the negative electrode can 302 are stacked in this order with the positive electrode can 301 positioned at the bottom, and the positive electrode can 301 and the negative electrode can 302 are subjected to pressure bonding with the gasket 303 located therebetween. In such a manner, the coin-type secondary battery 300 is manufactured.

When the positive electrode active material described in the above embodiment is used in the positive electrode 304, the coin-type secondary battery 300 with high charge and discharge capacity and excellent cycle performance can be obtained.

Here, a current flow in charging a secondary battery is described with reference to FIG. 16C. When a secondary battery using lithium is regarded as a closed circuit, movement of lithium ions and the current flow are in the same direction. Note that in the secondary battery using lithium, the anode and the cathode interchange in charge and discharge, and the oxidation reaction and the reduction reaction interchange; hence, an electrode with a high reaction potential is called a positive electrode and an electrode with a low reaction potential is called a negative electrode. For this reason, in this specification, the positive electrode is referred to as a “positive electrode” or a “plus electrode” and the negative electrode is referred to as a “negative electrode” or a “minus electrode” in all the cases where charge is performed, discharge is performed, a reverse pulse current is supplied, and a charge current is supplied. The use of the terms “anode” and “cathode” related to an oxidation reaction and a reduction reaction might cause confusion because the anode and the cathode interchange in charge and discharge. Thus, the terms “anode” and “cathode” are not used in this specification. If the term “anode” or “cathode” is used, it should be mentioned that the anode or the cathode is which of the one at the time of charge or the one at the time of discharge and corresponds to which of a positive (plus) electrode or a negative (minus) electrode.

Two terminals illustrated in FIG. 16C are connected to a charger, and the secondary battery 300 is charged. As the charge of the secondary battery 300 proceeds, a potential difference between electrodes increases.

### <Cylindrical Secondary Battery>

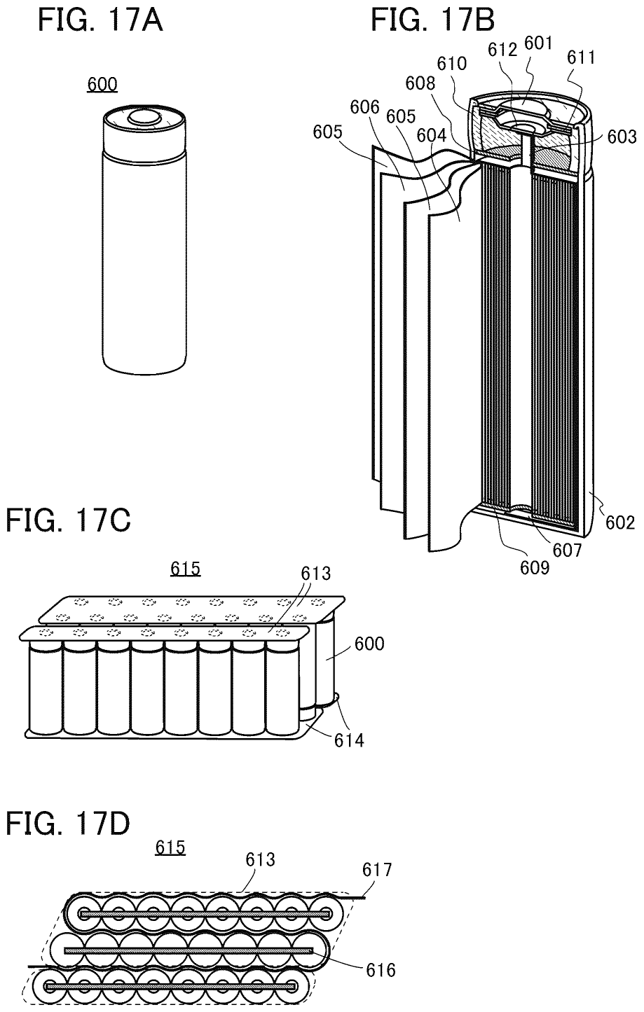

Next, an example of a cylindrical secondary battery is described with reference to FIG. 17. FIG. 17A shows an external view of a cylindrical secondary battery 600. FIG. 17B is a schematic cross-sectional view of the cylindrical secondary battery 600. The cylindrical secondary battery 600 includes, as illustrated in FIG. 17B, a positive electrode cap (battery lid) 601 on the top surface and a battery can (outer can) 602 on a side surface and a bottom surface. The positive electrode cap and the battery can (outer can) 602 are insulated from each other by a gasket (insulating gasket) 610.

Inside the battery can 602 having a hollow cylindrical shape, a battery element in which a strip-like positive electrode 604 and a strip-like negative electrode 606 are wound with a separator 605 located therebetween is provided. Although not illustrated, the battery element is wound around a center pin. One end of the battery can 602 is close and the other end thereof is open. For the battery can 602, a metal having corrosion resistance to an electrolyte, such as nickel, aluminum, or titanium, an alloy of such a metal, or an alloy of such a metal and another metal (e.g., stainless steel) can be used. The battery can 602 is preferably covered with nickel, aluminum, and/or the like in order to prevent corrosion due to the electrolyte. Inside the battery can 602, the battery element in which the positive electrode, the negative electrode, and the separator are wound is provided between a pair of insulating plates 608 and 609 that face each other. Furthermore, a nonaqueous electrolyte (not illustrated) is injected inside the battery can 602 provided with the battery element. As the nonaqueous electrolyte, a nonaqueous electrolyte that is similar to that of the coin-type secondary battery can be used.

Since a positive electrode and a negative electrode that are used for a cylindrical storage battery are wound, active materials are preferably formed on both surfaces of a current collector. A positive electrode terminal (positive electrode current collecting lead) 603 is connected to the positive electrode 604, and a negative electrode terminal (negative electrode current collecting lead) 607 is connected to the negative electrode 606. Both the positive electrode terminal 603 and the negative electrode terminal 607 can be formed using a metal material such as aluminum. The positive electrode terminal 603 and the negative electrode terminal 607 are resistance-welded to a safety valve mechanism 612 and the bottom of the battery can 602, respectively. The safety valve mechanism 612 is electrically connected to the positive electrode cap 601 through a PTC element (Positive Temperature Coefficient) 611. The safety valve mechanism 612 cuts off electrical connection between the positive electrode cap 601 and the positive electrode 604 when the internal pressure of the battery exceeds a predetermined threshold value. The PTC element 611, which serves as a thermally sensitive resistor whose resistance increases as temperature rises, limits the amount of current by increasing the resistance, in order to prevent abnormal heat generation. Barium titanate (BaTiO₃)-based semiconductor ceramics or the like can be used for the PTC element.

Furthermore, as illustrated in FIG. 17C, a plurality of secondary batteries 600 may be provided between a conductive plate 613 and a conductive plate 614 to form a module 615. The plurality of secondary batteries 600 may be connected in parallel, connected in series, or connected in series after being connected in parallel. With the module 615 including the plurality of secondary batteries 600, large electric power can be extracted.

FIG. 17D is a top view of the module 615. The conductive plate 613 is shown by a dotted line for clarity of the diagram. As illustrated in FIG. 17D, the module 615 may include a wiring 616 electrically connecting the plurality of secondary batteries 600 with each other. It is possible to provide the conductive plate over the wiring 616 to overlap with each other. In addition, a temperature control device 617 may be provided between the plurality of secondary batteries 600. The secondary batteries 600 can be cooled with the temperature control device 617 when overheated, whereas the secondary batteries 600 can be heated with the temperature control device 617 when cooled too much. Thus, the performance of the module 615 is unlikely to be affected by the outside temperature. A heating medium included in the temperature control device 617 preferably has an insulating property and incombustibility.

When the positive electrode active material described in the above embodiment is used in the positive electrode 604, the cylindrical secondary battery 600 with high charge and discharge capacity and excellent cycle performance can be obtained.

### <Structure Examples of Secondary Battery>

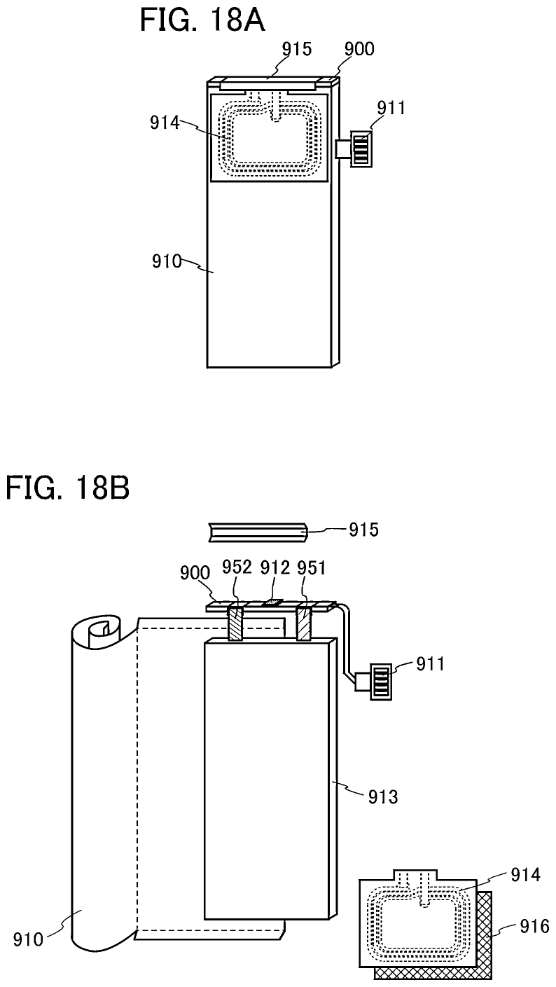

Other structure examples of secondary batteries are described with reference to FIG. 18 to FIG. 22.

FIG. 18A and FIG. 18B are external views of a battery pack. The battery pack includes a secondary battery 913 and a circuit board 900. A secondary battery 913 is connected to an antenna 914 through a circuit board 900. A label 910 is attached to the secondary battery 913. In addition, as illustrated in FIG. 18B, the secondary battery 913 is connected to a terminal 951 and a terminal 952. The circuit board 900 is fixed with a seal 915.

The circuit board 900 includes a terminal 911 and a circuit 912. The terminal 911 is connected to the terminal 951, the terminal 952, the antenna 914, and the circuit 912. Note that a plurality of terminals 911 may be provided to serve as a control signal input terminal, a power supply terminal, and the like.

The circuit 912 may be provided on the rear surface of the circuit board 900. Note that the shape of the antenna 914 is not limited to coil shapes, and may be a linear shape or a plate shape, for example. An antenna such as a planar antenna, an aperture antenna, a traveling-wave antenna, an EH antenna, a magnetic-field antenna, or a dielectric antenna may be used. Alternatively, the antenna 914 may be a flat-plate conductor. The flat-plate conductor can serve as one of conductors for electric field coupling. That is, the antenna 914 may serve as one of two conductors of a capacitor. Thus, electric power can be transmitted and received not only by an electromagnetic field or a magnetic field but also by an electric field.

The battery pack includes a layer 916 between the antenna 914 and the secondary battery 913. The layer 916 has a function of blocking an electromagnetic field by the secondary battery 913, for example. As the layer 916, for example, a magnetic body can be used.

Note that the structure of the battery pack is not limited to that in FIG. 18.

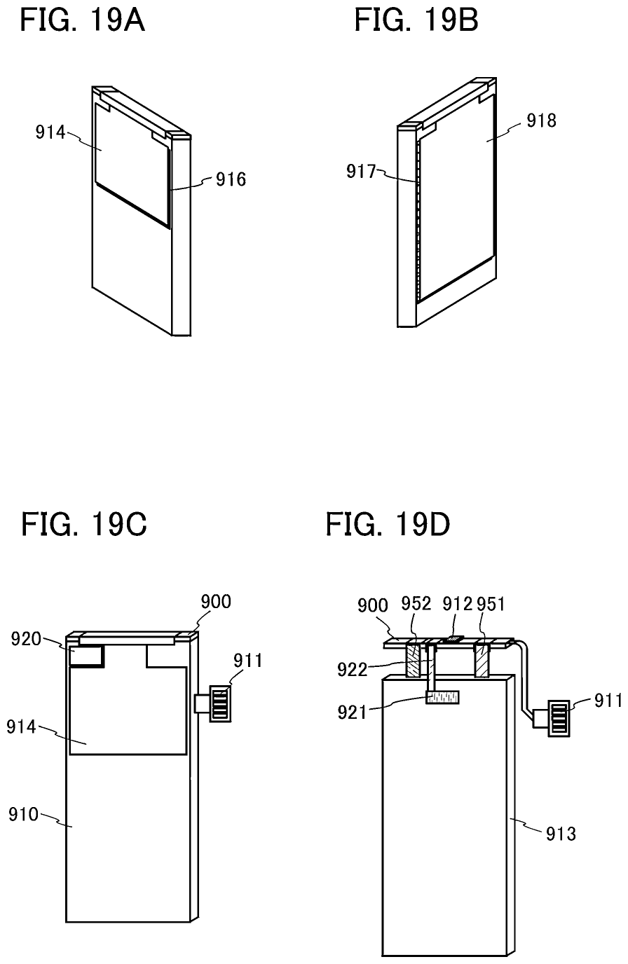

For example, as illustrated in FIG. 19A and FIG. 19B, two opposite surfaces of the secondary battery 913 illustrated in FIG. 18A and FIG. 18B may be provided with respective antennas. FIG. 19A is an external view seen from one side of the opposite surfaces, and FIG. 19A is an external view seen from the other side of the opposite surfaces. Note that for portions similar to those of the secondary battery illustrated in FIG. 18A and FIG. 18B, the description of the secondary battery illustrated in FIG. 18A and FIG. 18B can be appropriately referred to.

As illustrated in FIG. 19A, the antenna 914 is provided on one of the opposite surfaces of the secondary battery 913 with the layer 916 located therebetween, and as illustrated in FIG. 19B, an antenna 918 is provided on the other of the opposite surfaces of the secondary battery 913 with a layer 917 located therebetween. The layer 917 has a function of blocking an electromagnetic field by the secondary battery 913, for example. As the layer 917, for example, a magnetic body can be used.

With the above structure, both of the antenna 914 and the antenna 918 can be increased in size. The antenna 918 has a function of communicating data with an external device, for example. An antenna with a shape that can be used for the antenna 914, for example, can be used as the antenna 918. As a system for communication using the antenna 918 between the secondary battery and another device, a response method that can be used between the secondary battery and another device, such as NFC (near field communication), can be employed.

Alternatively, as illustrated in FIG. 19C, the secondary battery 913 illustrated in FIG. 18A and FIG. 18B may be provided with a display device 920. The display device 920 is electrically connected to the terminal 911. Note that the label 910 is not necessarily provided in a portion where the display device 920 is provided. Note that for portions similar to those of the secondary battery illustrated in FIG. 18A and FIG. 18B, the description of the secondary battery illustrated in FIG. 18A and FIG. 18B can be appropriately referred to.

The display device 920 may display, for example, an image showing whether charge is being carried out, an image showing the amount of stored power, or the like. As the display device 920, electronic paper, a liquid crystal display device, an electroluminescent (EL) display device, or the like can be used. For example, the use of electronic paper can reduce power consumption of the display device 920.

Alternatively, as illustrated in FIG. 19D, the secondary battery 913 illustrated in FIG. 18A and FIG. 18B may be provided with a sensor 921. The sensor 921 is electrically connected to the terminal 911 via a terminal 922. Note that for portions similar to those of the secondary battery illustrated in FIG. 18A and FIG. 18B, the description of the secondary battery illustrated in FIG. 18A and FIG. 18B can be appropriately referred to.

The sensor 921 has a function of measuring, for example, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays. With the sensor 921, for example, data on an environment (e.g., temperature) where the secondary battery is placed can be detected and stored in a memory inside the circuit 912.

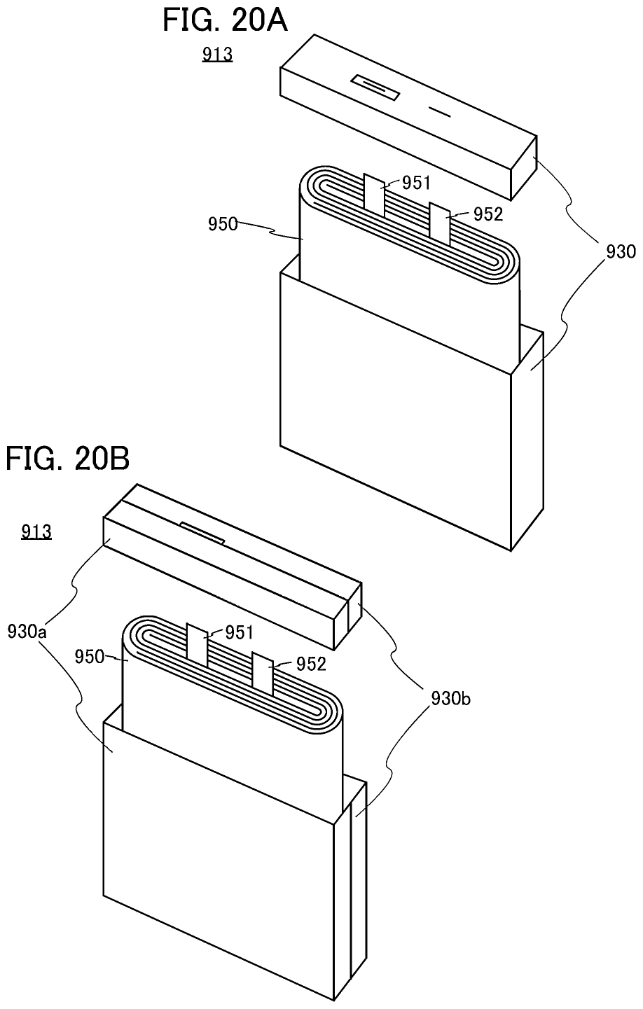

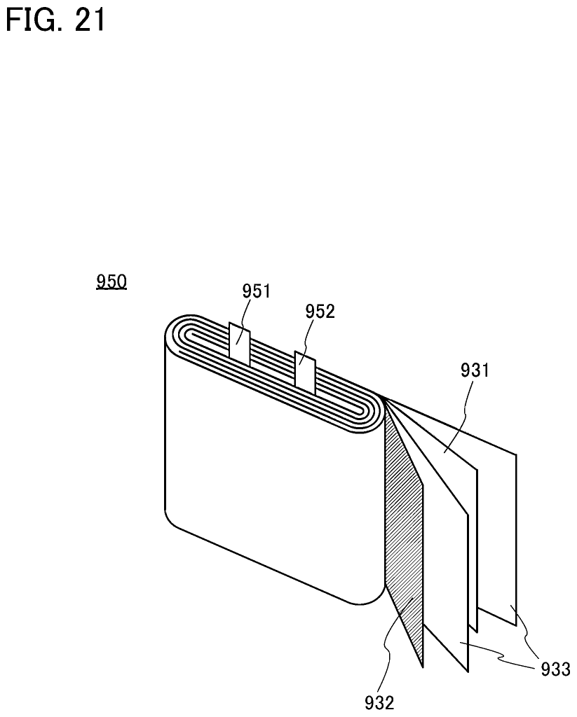

Furthermore, structure examples of the secondary battery 913 are described with reference to FIG. 20 and FIG. 21.

The secondary battery 913 illustrated in FIG. 20A includes a wound body 950 provided with the terminal 951 and the terminal 952 inside a housing 930. The wound body 950 is soaked in an electrolyte inside the housing 930. The terminal 952 is in contact with the housing 930. The use of an insulator or the like prevents contact between the terminal 951 and the housing 930. Note that in FIG. 20A, the housing 930 divided into pieces is illustrated for convenience; however, in the actual structure, the wound body 950 is covered with the housing 930 and the terminal 951 and the terminal 952 extend to the outside of the housing 930. For the housing 930, a metal material (e.g., aluminum) or a resin material can be used.

Note that as illustrated in FIG. 20B, the housing 930 illustrated in FIG. 20A may be formed using a plurality of materials. For example, in the secondary battery 913 illustrated in FIG. 20B, a housing 930a and a housing 930b are bonded to each other, and the wound body 950 is provided in a region surrounded by the housing 930a and the housing 930b.

For the housing 930a, an insulating material such as an organic resin can be used. In particular, when a material such as an organic resin is used for the side on which an antenna is formed, blocking of an electric field from the secondary battery 913 can be inhibited. When an electric field is not significantly blocked by the housing 930a, an antenna such as the antenna 914 may be provided inside the housing 930a. For the housing 930b, a metal material can be used, for example.

FIG. 21 illustrates the structure of the wound body 950. The wound body 950 includes a negative electrode 931, a positive electrode 932, and separators 933. The wound body 950 is obtained by winding a sheet of a stack in which the negative electrode 931 overlaps with the positive electrode 932 with the separator 933 provided therebetween. Note that a plurality of stacks each including the negative electrode 931, the positive electrode 932, and the separator 933 may be further stacked.

The negative electrode 931 is connected to the terminal 911 illustrated in FIG. 18 via one of the terminal 951 and the terminal 952. The positive electrode 932 is connected to the terminal 911 illustrated in FIG. 18 via the other of the terminal 951 and the terminal 952.

When the positive electrode active material described in the above embodiment is used in the positive electrode 932, the secondary battery 913 with high charge and discharge capacity and excellent cycle performance can be obtained.

### <Laminated Secondary Battery>

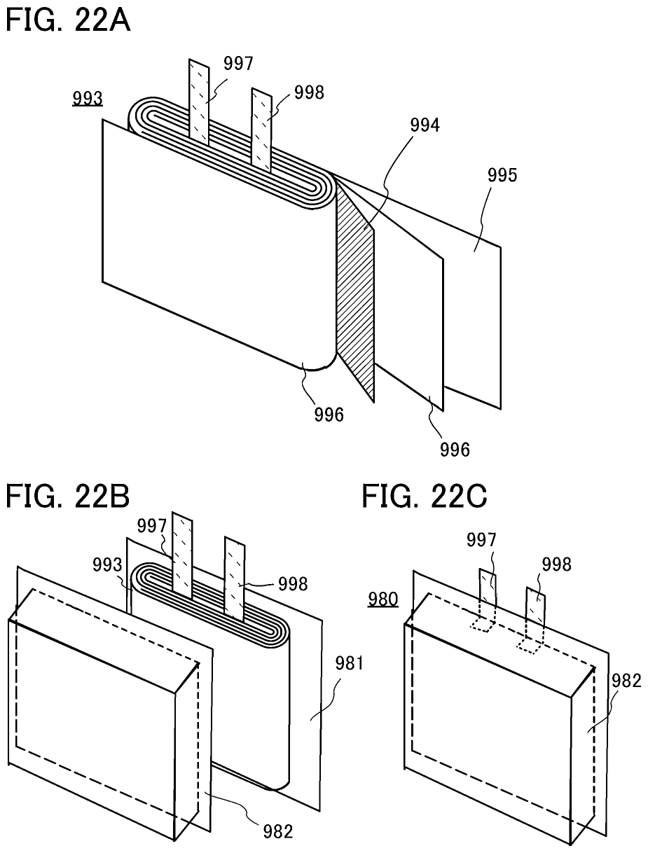

Next, an example of a laminated secondary battery is described with reference to FIG. 22 to FIG. 31. When the laminated secondary battery has flexibility and is used in an electronic device at least part of which is flexible, the secondary battery can be bent as the electronic device is bent.

A laminated secondary battery 980 is described with reference to FIG. 22. The laminated secondary battery 980 includes a wound body 993 illustrated in FIG. 22A. The wound body 993 includes a negative electrode 994, a positive electrode 995, and separators 996. The wound body 993 is, like the wound body 950 illustrated in FIG. 21, obtained by winding a sheet of a stack in which the negative electrode 994 overlaps with the positive electrode 995 with the separator 996 provided therebetween.

Note that the number of stacks each including the negative electrode 994, the positive electrode 995, and the separator 996 may be designed as appropriate depending on required charge and discharge capacity and element volume. The negative electrode 994 is connected to a negative electrode current collector (not illustrated) via one of a lead electrode 997 and a lead electrode 998. The positive electrode 995 is connected to a positive electrode current collector (not illustrated) via the other of the lead electrode 997 and the lead electrode 998.

As illustrated in FIG. 22B, the above-described wound body 993 is packed in a space formed by bonding a film 981 and a film 982 having a depressed portion that serve as exterior bodies by thermocompression bonding or the like, whereby the secondary battery 980 as illustrated in FIG. 22C can be formed. The wound body 993 includes the lead electrode 997 and the lead electrode 998, and is soaked in an electrolyte inside the film 981 and the film 982 having a depressed portion.

For the film 981 and the film 982 having a depressed portion, a metal material such as aluminum or a resin material can be used, for example. With the use of a resin material for the film 981 and the film 982 having a depressed portion, the film 981 and the film 982 having a depressed portion can be changed in their forms when external force is applied; thus, a flexible storage battery can be manufactured.

Although FIG. 22B and FIG. 22C show an example of using two films, the wound body 993 may be placed in a space formed by bending one film.

When the positive electrode active material described in the above embodiment is used in the positive electrode 995, the secondary battery 980 with high charge and discharge capacity and excellent cycle performance can be obtained.

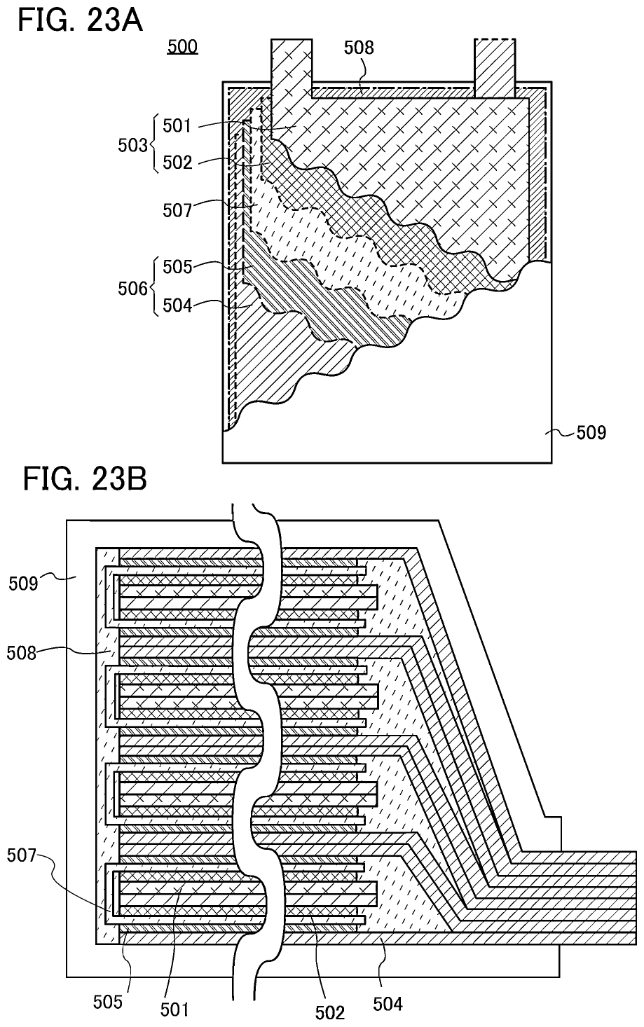

In FIG. 22, an example in which the secondary battery 980 includes a wound body in a space formed by films serving as exterior bodies is described; however, as illustrated in FIG. 23, a secondary battery may include a plurality of strip-shaped positive electrodes, a plurality of strip-shaped separators, and a plurality of strip-shaped negative electrodes in a space formed by films serving as exterior bodies, for example.

The laminated secondary battery 500 illustrated in FIG. 23A includes the positive electrode 503 including a positive electrode current collector 501 and a positive electrode active material layer 502, the negative electrode 506 including a negative electrode current collector 504 and a negative electrode active material layer 505, the separator 507, the electrolyte 508, and the exterior body 509. The separator 507 is provided between the positive electrode 503 and the negative electrode 506 in the exterior body 509. The exterior body 509 is filled with the electrolyte 508. The electrolyte described in Embodiment 3 can be used as the electrolyte 508.

In the laminated secondary battery 500 illustrated in FIG. 23A, the positive electrode current collector 501 and the negative electrode current collector 504 also serve as terminals for electrical contact with the outside. For this reason, the positive electrode current collector 501 and the negative electrode current collector 504 may be arranged so that part of the positive electrode current collector 501 and part of the negative electrode current collector 504 are exposed to the outside of the exterior body 509. Alternatively, without exposing the positive electrode current collector 501 and the negative electrode current collector 504 from the exterior body 509 to the outside, a lead electrode may be used, and the lead electrode and the positive electrode current collector 501 or the negative electrode current collector 504 may be bonded by ultrasonic welding so that the lead electrode is exposed to the outside.

As the exterior body 509 of the laminated secondary battery 500, for example, a laminate film having a three-layer structure can be employed in which a highly flexible metal thin film of aluminum, stainless steel, copper, nickel, or the like is provided over a film formed of a material such as polyethylene, polypropylene, polycarbonate, ionomer, or polyamide, and an insulating synthetic resin film of a polyamide-based resin, a polyester-based resin, or the like is provided as the outer surface of the exterior body over the metal thin film.

FIG. 23B shows an example of a cross-sectional structure of the laminated secondary battery 500. FIG. 23A shows an example in which only two current collectors are included for simplicity, but actually, a plurality of electrode layers are included as illustrated in FIG. 23B.

In FIG. 23B, the number of electrode layers is 16, for example. Note that the secondary battery 500 has flexibility even though the number of electrode layers is set to 16. FIG. 23B illustrates a structure including 8 layers of negative electrode current collectors 504 and 8 layers of positive electrode current collectors 501, i.e., 16 layers in total. Note that FIG. 23B illustrates a cross section of the lead portion of the negative electrode, and the 8 layers of the negative electrode current collectors 504 are bonded to each other by ultrasonic welding. It is needless to say that the number of electrode layers is not limited to 16, and may be more than 16 or less than 16. With a large number of electrode layers, the secondary battery can have high charge and discharge capacity. In contrast, with a small number of electrode layers, the secondary battery can have small thickness and high flexibility.

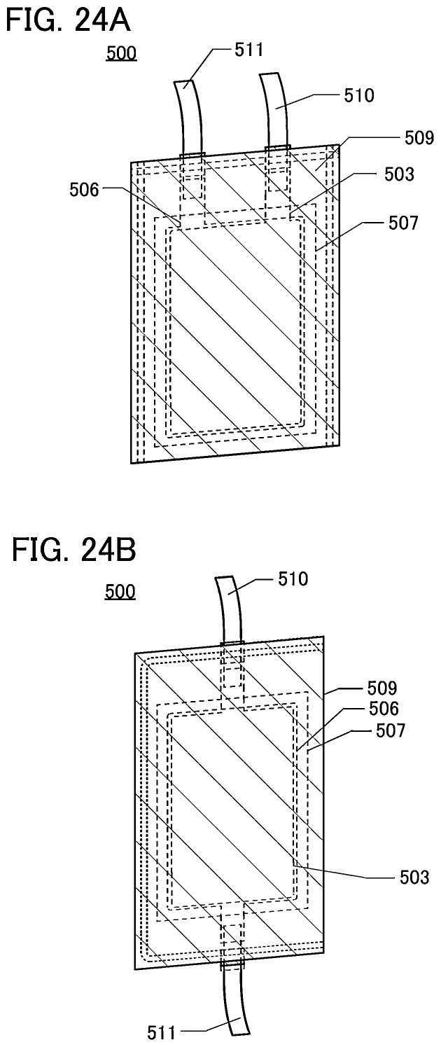

FIG. 24A and FIG. 24B each show an example of the external view of the laminated secondary battery 500. In FIG. 24A and FIG. 24B, the positive electrode 503, the negative electrode 506, the separator 507, the exterior body 509, the positive electrode lead electrode 510, and the negative electrode lead electrode 511 are included.

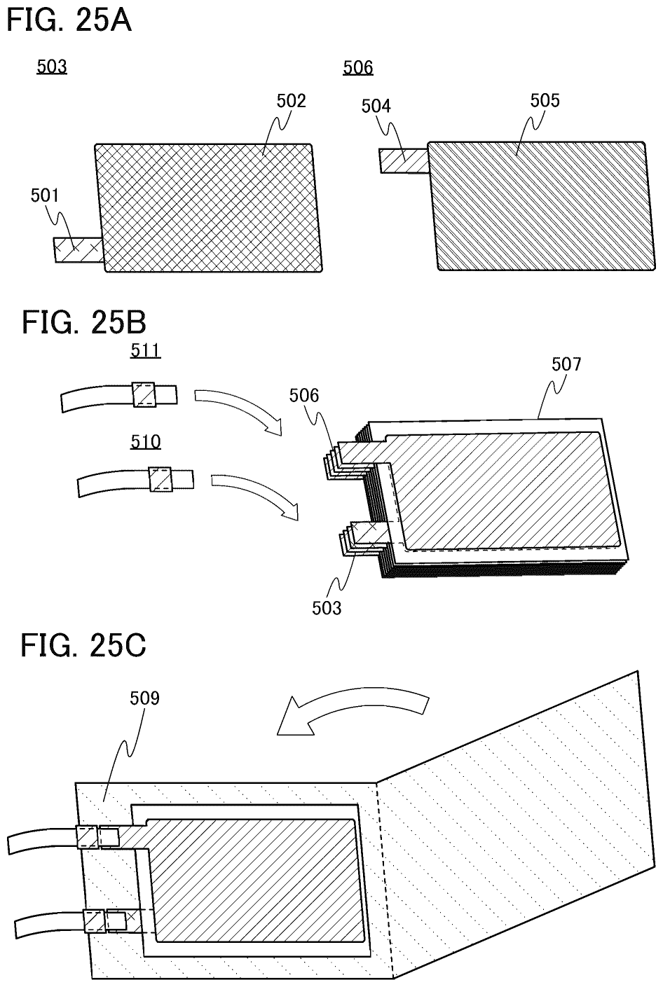

FIG. 25A illustrates external views of the positive electrode 503 and the negative electrode 506. The positive electrode 503 includes the positive electrode current collector 501, and the positive electrode active material layer 502 is formed on a surface of the positive electrode current collector 501. The positive electrode 503 also includes a region where the positive electrode current collector 501 is partly exposed (hereinafter, referred to as a tab region). The negative electrode 506 includes the negative electrode current collector 504, and the negative electrode active material layer 505 is formed on a surface of the negative electrode current collector 504. The negative electrode 506 also includes a region where the negative electrode current collector 504 is partly exposed, that is, a tab region. The areas and/or the shapes of the tab regions included in the positive electrode and the negative electrode are not limited to those illustrated in FIG. 25A.

### <Method for Manufacturing Laminated Secondary Battery>

Here, an example of a method for manufacturing the laminated secondary battery whose external view is illustrated in FIG. 24A is described with reference to FIG. 25B and FIG. 25C.

First, the negative electrode 506, the separator 507, and the positive electrode 503 are stacked. FIG. 25B illustrates a stack including the negative electrode 506, the separator 507, and the positive electrode 503. Here, an example in which 5 negative electrodes and 4 positive electrodes are used is shown. Next, the tab regions of the positive electrodes 503 are bonded to each other, and the tab region of the positive electrode on the outermost surface and the positive electrode lead electrode 510 are bonded to each other. The bonding can be performed by ultrasonic welding, for example. In a similar manner, the tab regions of the negative electrodes 506 are bonded to each other, and the tab region of the negative electrode on the outermost surface and the negative electrode lead electrode 511 are bonded to each other.

After that, the negative electrode 506, the separator 507, and the positive electrode 503 are placed over the exterior body 509.

Subsequently, the exterior body 509 is folded along a portion shown by a dashed line as illustrated in FIG. 25C. Then, the outer edges of the exterior body 509 are bonded to each other. The bonding can be performed by thermocompression bonding, for example. At this time, an unbonded region (hereinafter referred to as an inlet) is provided for part (or one side) of the exterior body 509 so that the electrolyte 508 can be put later.

Next, the electrolyte 508 (not illustrated) is introduced into the exterior body 509 from the inlet of the exterior body 509. The electrolyte 508 is preferably introduced in a reduced pressure atmosphere or in an inert gas atmosphere. Lastly, the inlet is bonded. In the above manner, the laminated secondary battery 500 can be manufactured.

When the positive electrode active material described in the above embodiment is used in the positive electrode 503, the secondary battery 500 with high charge and discharge capacity and excellent cycle performance can be obtained.

In an all-solid-state battery, the contact state of the inside interfaces can be kept favorable by applying a predetermined pressure in the direction of stacking positive electrodes and negative electrodes. By applying a predetermined pressure in the direction of stacking positive electrodes and negative electrodes, expansion in the stacking direction due to charge and discharge of the all-solid-state battery can be suppressed, and the reliability of the all-solid-state battery can be improved.

This embodiment can be used in appropriate combination with the other embodiments.

### Embodiment 5

In this embodiment, examples of electronic devices each including the secondary battery of one embodiment of the present invention are described.

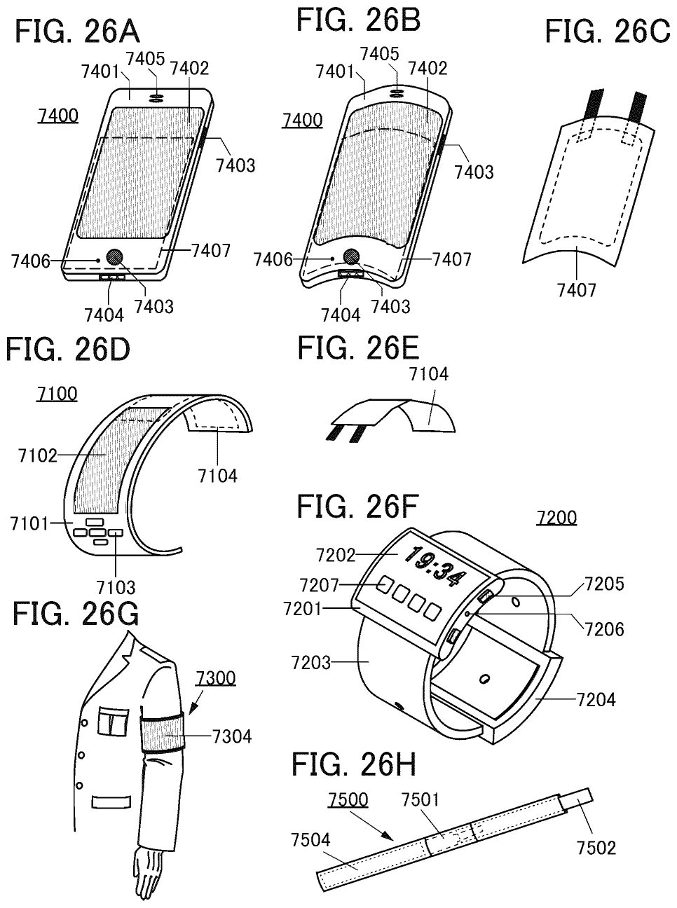

First, FIG. 26A to FIG. 26G show examples of electronic devices including the bendable secondary battery described in the above embodiment. Examples of electronic devices each including a bendable secondary battery include television sets (also referred to as televisions or television receivers), monitors of computers or the like, digital cameras, digital video cameras, digital photo frames, mobile phones (also referred to as cellular phones or mobile phone devices), portable game machines, portable information terminals, audio reproducing devices, and large game machines such as pachinko machines.

Furthermore, a flexible secondary battery can be incorporated along a curved inside or outside wall surface of a house and/or a building or a curved interior or exterior surface of an automobile, for example.

FIG. 26A shows an example of a mobile phone. A mobile phone 7400 is provided with a display portion 7402 incorporated in a housing 7401, operation buttons 7403, an external connection port 7404, a speaker 7405, a microphone 7406, and the like. Note that the mobile phone 7400 includes a secondary battery 7407. When the secondary battery of one embodiment of the present invention is used as the secondary battery 7407, a lightweight mobile phone with a long lifetime can be provided.

FIG. 26B illustrates the mobile phone 7400 that is curved. When the whole mobile phone 7400 is curved by external force, the secondary battery 7407 provided therein is also curved. FIG. 26C illustrates the bent secondary battery 7407. The secondary battery 7407 is a thin storage battery. The secondary battery 7407 is fixed in a state of being bent. Note that the secondary battery 7407 includes a lead electrode electrically connected to a current collector. The current collector is, for example, copper foil, and partly alloyed with gallium; thus, adhesion between the current collector and an active material layer in contact with the current collector is improved and the secondary battery 7407 can have high reliability even in a state of being bent.

FIG. 26D shows an example of a bangle display device. A portable display device 7100 includes a housing 7101, a display portion 7102, operation buttons 7103, and a secondary battery 7104. FIG. 26E illustrates the bent secondary battery 7104. When the display device is worn on a user's arm while the secondary battery 7104 is bent, the housing changes its shape and the curvature of part or the whole of the secondary battery 7104 is changed. Note that the bending condition of a curve at a given point that is represented by a value of the radius of a corresponding circle is referred to as the radius of curvature, and the reciprocal of the radius of curvature is referred to as curvature. Specifically, part or the whole of the housing or the main surface of the secondary battery 7104 is changed in the range of radius of curvature from 40 mm or more to 150 mm or less. When the radius of curvature at the main surface of the secondary battery 7104 is in the range from 40 mm or more to 150 mm or less, the reliability can be kept high. When the secondary battery of one embodiment of the present invention is used as the secondary battery 7104, a lightweight portable display device with a long lifetime can be provided.

FIG. 26F shows an example of a watch-type portable information terminal. A portable information terminal 7200 includes a housing 7201, a display portion 7202, a band 7203, a buckle 7204, an operation button 7205, an input/output terminal 7206, and the like.

The portable information terminal 7200 is capable of executing a variety of applications such as mobile phone calls, e-mailing, viewing and editing texts, music reproduction, Internet communication, and a computer game.

The display surface of the display portion 7202 is curved, and images can be displayed on the curved display surface. In addition, the display portion 7202 includes a touch sensor, and operation can be performed by touching the screen with a finger, a stylus, or the like. For example, by touching an icon 7207 displayed on the display portion 7202, application can be started.

With the operation button 7205, a variety of functions such as time setting, power on/off, on/off of wireless communication, setting and cancellation of a silent mode, and setting and cancellation of a power saving mode can be performed. For example, the functions of the operation button 7205 can be set freely by setting the operating system incorporated in the portable information terminal 7200.

The portable information terminal 7200 can perform near field communication that is standardized communication. For example, mutual communication between the portable information terminal 7200 and a headset capable of wireless communication enables hands-free calling.

The portable information terminal 7200 includes the input/output terminal 7206, and data can be directly transmitted to and received from another information terminal via a connector. In addition, charge via the input/output terminal 7206 is possible. Note that the charging operation may be performed by wireless power feeding without using the input/output terminal 7206.

The display portion 7202 of the portable information terminal 7200 includes the secondary battery of one embodiment of the present invention. When the secondary battery of one embodiment of the present invention is used, a lightweight portable information terminal with a long lifetime can be provided. For example, the secondary battery 7104 illustrated in FIG. 26E can be provided in the housing 7201 while being curved, or the secondary battery 7104 illustrated in FIG. 26E can be provided in the band 7203 such that it can be curved.

The portable information terminal 7200 preferably includes a sensor. As the sensor, for example, a human body sensor such as a fingerprint sensor, a pulse sensor, or a temperature sensor, a touch sensor, a pressure sensitive sensor, or an acceleration sensor is preferably mounted.

FIG. 26G shows an example of an armband display device. A display device 7300 includes a display portion 7304 and the secondary battery of one embodiment of the present invention. The display device 7300 can include a touch sensor in the display portion 7304 and can serve as a portable information terminal.

The display surface of the display portion 7304 is curved, and images can be displayed on the curved display surface. A display state of the display device 7300 can be changed by, for example, near field communication that is standardized communication.

The display device 7300 includes an input/output terminal, and data can be directly transmitted to and received from another information terminal via a connector. In addition, charge via the input/output terminal is possible. Note that the charging operation may be performed by wireless power feeding without using the input/output terminal.

When the secondary battery of one embodiment of the present invention is used as the secondary battery included in the display device 7300, a lightweight display device with a long lifetime can be provided.

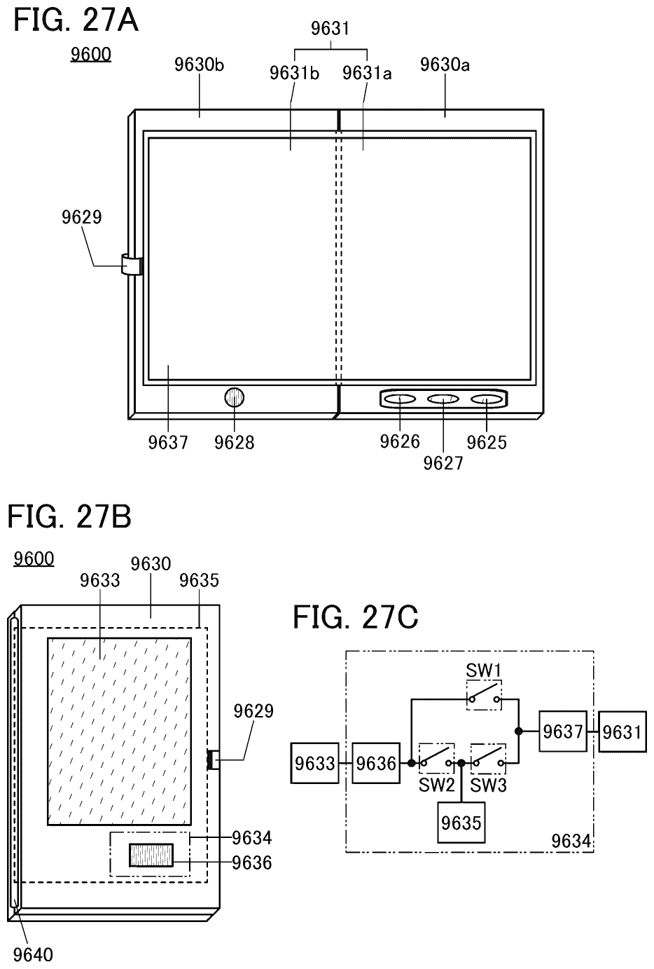

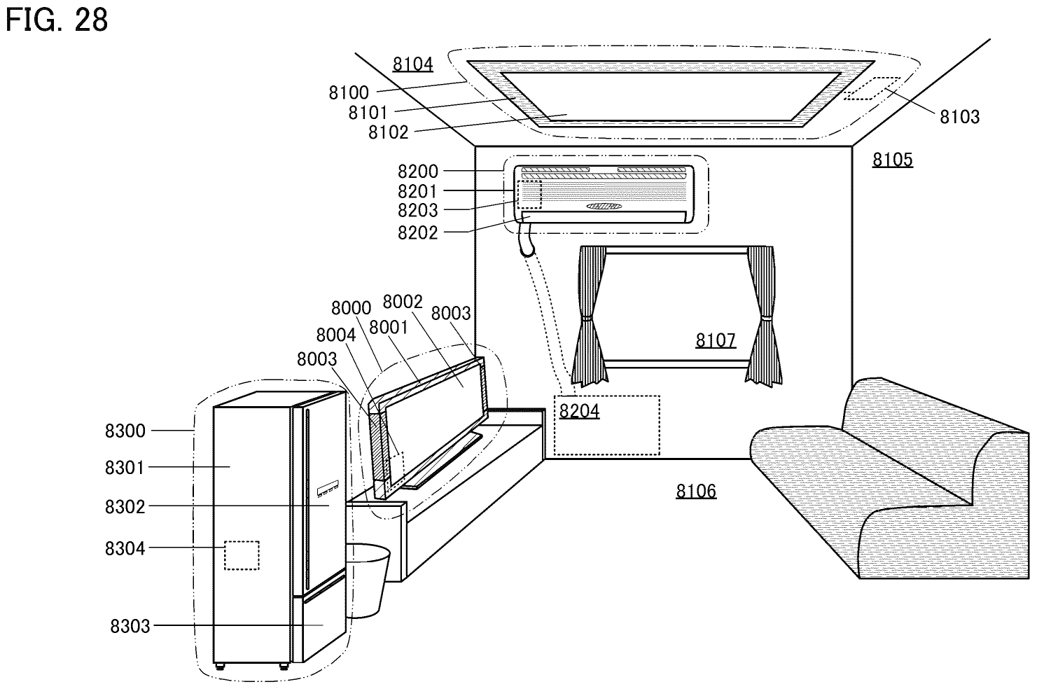

Examples of electronic devices each including the secondary battery with high charge and discharge capacity and excellent cycle performance described in the above embodiment are described with reference to FIG. 26H, FIG. 27, and FIG. 28.

When the secondary battery of one embodiment of the present invention is used as a secondary battery of a daily electronic device, a lightweight product with a long lifetime can be provided. Examples of the daily electronic device include an electric toothbrush, an electric shaver, and electric beauty equipment. As secondary batteries of these products, small and lightweight stick type secondary batteries with high charge and discharge capacity are desired in consideration of handling ease for users.

FIG. 26H is a perspective view of a device called a cigarette smoking device (electronic cigarette). In FIG. 26H, an electronic cigarette 7500 includes an atomizer 7501 including a heating element, a secondary battery 7504 that supplies power to the atomizer, and a cartridge 7502 including a liquid supply bottle, a sensor, and the like. To improve safety, a protection circuit that prevents overcharge and overdischarge of the secondary battery 7504 may be electrically connected to the secondary battery 7504. The secondary battery 7504 illustrated in FIG. 26H includes an external terminal for connection to a charger. When the electronic cigarette 7500 is held, the secondary battery 7504 is a tip portion; thus, it is preferable that the secondary battery 7504 have a short total length and be lightweight. With the secondary battery of one embodiment of the present invention, which has high charge and discharge capacity and excellent cycle performance, the small and lightweight electronic cigarette 7500 that can be used for a long time over a long period can be provided.

Next, FIG. 27A and FIG. 27B show an example of a tablet terminal that can be folded in half. A tablet terminal 9600 illustrated in FIG. 27A and FIG. 27B includes a housing 9630a, a housing 9630b, a movable portion 9640 connecting the housing 9630a and the housing 9630b to each other, a display portion 9631 including a display portion 9631a and a display portion 9631b, a switch 9625 to a switch 9627, a fastener 9629, and an operation switch 9628. A flexible panel is used for the display portion 9631, whereby a tablet terminal with a larger display portion can be provided. FIG. 27A illustrates the tablet terminal 9600 that is opened, and FIG. 27B illustrates the tablet terminal 9600 that is closed.

The tablet terminal 9600 includes a power storage unit 9635 inside the housing 9630a and the housing 9630b. The power storage unit 9635 is provided across the housing 9630a and the housing 9630b, passing through the movable portion 9640.

The entire region or part of the region of the display portion 9631 can be a touch panel region, and data can be input by touching text, an input form, an image including an icon, and the like displayed on the region. For example, it is possible that keyboard buttons are displayed on the entire display portion 9631a on the housing 9630a side, and data such as text or an image is displayed on the display portion 9631b on the housing 9630b side.

It is possible that a keyboard is displayed on the display portion 9631b on the housing 9630b side, and data such as text or an image is displayed on the display portion 9631a on the housing 9630a side. Furthermore, it is possible that a switching button for showing/hiding a keyboard on a touch panel is displayed on the display portion 9631 and the button is touched with a finger, a stylus, and the like to display a keyboard on the display portion 9631.

Touch input can be performed concurrently in a touch panel region in the display portion 9631a on the housing 9630a side and a touch panel region in the display portion 9631b on the housing 9630b side.

The switch 9625 to the switch 9627 may function not only as an interface for operating the tablet terminal 9600 but also as an interface that can switch various functions. For example, at least one of the switch 9625 to the switch 9627 may function as a switch for switching power on/off of the tablet terminal 9600. For another example, at least one of the switch 9625 to the switch 9627 may have a function of switching the display orientation between a portrait mode and a landscape mode and a function of switching display between monochrome display and color display. For another example, at least one of the switch 9625 to the switch 9627 may have a function of adjusting the luminance of the display portion 9631. The luminance of the display portion 9631 can be optimized in accordance with the amount of external light in use of the tablet terminal 9600 detected by an optical sensor incorporated in the tablet terminal 9600. Note that another sensing device including a sensor for measuring inclination, such as a gyroscope sensor or an acceleration sensor, may be incorporated in the tablet terminal, in addition to the optical sensor.

FIG. 27A shows an example in which the display portion 9631a on the housing 9630a side and the display portion 9631b on the housing 9630b side have substantially the same display area; however, there is no particular limitation on the display areas of the display portion 9631a and the display portion 9631b, and the display portions may have different sizes or different display quality. For example, one may be a display panel that can display higher-definition images than the other.

The tablet terminal 9600 is folded in half in FIG. 27B. The tablet terminal 9600 includes a housing 9630, a solar cell 9633, and a charge and discharge control circuit 9634 including a DCDC converter 9636. The secondary battery of one embodiment of the present invention is used as the power storage unit 9635.

Note that as described above, the tablet terminal 9600 can be folded in half, and thus can be folded when not in use such that the housing 9630a and the housing 9630b overlap with each other. By the folding, the display portion 9631 can be protected, which increases the durability of the tablet terminal 9600. With the power storage unit 9635 including the secondary battery of one embodiment of the present invention, which has high charge and discharge capacity and excellent cycle performance, the tablet terminal 9600 that can be used for a long time over a long period can be provided.

The tablet terminal 9600 illustrated in FIG. 27A and FIG. 27B can also have a function of displaying various kinds of data (e.g., a still image, a moving image, and a text image), a function of displaying a calendar, a date, or the time on the display portion, a touch-input function of operating or editing data displayed on the display portion by touch input, a function of controlling processing by various kinds of software (programs), and the like.

The solar cell 9633, which is attached on the surface of the tablet terminal 9600, can supply electric power to a touch panel, a display portion, a video signal processing portion, and the like. Note that the solar cell 9633 can be provided on one surface or both surfaces of the housing 9630 and the power storage unit 9635 can be charged efficiently. The use of a lithium-ion battery as the power storage unit 9635 brings an advantage such as a reduction in size.

The structure and operation of the charge and discharge control circuit 9634 illustrated in FIG. 27B are described with reference to a block diagram in FIG. 27C. The solar cell 9633, the power storage unit 9635, the DCDC converter 9636, a converter 9637, switches SW1 to SW3, and the display portion 9631 are illustrated in FIG. 27C, and the power storage unit 9635, the DCDC converter 9636, the converter 9637, and the switches SW1 to SW3 correspond to the charge and discharge control circuit 9634 illustrated in FIG. 27B.

First, an operation example in which electric power is generated by the solar cell 9633 using external light is described. The voltage of electric power generated by the solar cell is raised or lowered by the DCDC converter 9636 to a voltage for charging the power storage unit 9635. When the display portion 9631 is operated with the electric power from the solar cell 9633, the switch SW1 is turned on and the voltage is raised or lowered by the converter 9637 to a voltage needed for the display portion 9631. When display on the display portion 9631 is not performed, the switch SW1 is turned off and the switch SW2 is turned on, so that the power storage unit 9635 is charged.

Note that the solar cell 9633 is described as an example of a power generation unit; however, one embodiment of the present invention is not limited to this example. The power storage unit 9635 may be charged using another power generation unit such as a piezoelectric element or a thermoelectric conversion element (Peltier element). For example, the charge may be performed with a non-contact power transmission module that performs charge by transmitting and receiving power wirelessly (without contact), or with a combination of other charge units.

FIG. 28 illustrates other examples of electronic devices. In FIG. 28, a display device 8000 is an example of an electronic device including a secondary battery 8004 of one embodiment of the present invention. Specifically, the display device 8000 corresponds to a display device for TV broadcast reception and includes a housing 8001, a display portion 8002, speaker portions 8003, the secondary battery 8004, and the like. The secondary battery 8004 of one embodiment of the present invention is provided in the housing 8001. The display device 8000 can be supplied with electric power from a commercial power supply and can use electric power stored in the secondary battery 8004. Thus, the display device 8000 can be operated with the use of the secondary battery 8004 of one embodiment of the present invention as an uninterruptible power supply even when electric power cannot be supplied from a commercial power supply due to power failure or the like.

A semiconductor display device such as a liquid crystal display device, a light-emitting device in which a light-emitting element such as an organic EL element is provided in each pixel, an electrophoresis display device, a DMD (Digital Micromirror Device), a PDP (Plasma Display Panel), or an FED (Field Emission Display) can be used for the display portion 8002.

Note that the display device includes, in its category, all of information display devices for personal computers, advertisement displays, and the like besides information display devices for TV broadcast reception.

In FIG. 28, an installation lighting device 8100 is an example of an electronic device including a secondary battery 8103 of one embodiment of the present invention. Specifically, the lighting device 8100 includes a housing 8101, a light source 8102, the secondary battery 8103, and the like. Although FIG. 28 illustrates the case where the secondary battery 8103 is provided in a ceiling 8104 on which the housing 8101 and the light source 8102 are installed, the secondary battery 8103 may be provided in the housing 8101. The lighting device 8100 can be supplied with electric power from a commercial power supply and can use electric power stored in the secondary battery 8103. Thus, the lighting device 8100 can be operated with the use of the secondary battery 8103 of one embodiment of the present invention as an uninterruptible power supply even when electric power cannot be supplied from a commercial power supply due to power failure or the like.

Note that although the installation lighting device 8100 provided in the ceiling 8104 is illustrated in FIG. 28 as an example, the secondary battery of one embodiment of the present invention can be used in an installation lighting device provided in, for example, a side wall 8105, a floor 8106, or a window 8107 other than the ceiling 8104, and can be used in a tabletop lighting device or the like.

As the light source 8102, an artificial light source that emits light artificially by using electric power can be used. Specifically, an incandescent lamp, a discharge lamp such as a fluorescent lamp, and light-emitting elements such as an LED and an organic EL element are given as examples of the artificial light source.

In FIG. 28, an air conditioner including an indoor unit 8200 and an outdoor unit 8204 is an example of an electronic device including a secondary battery 8203 of one embodiment of the present invention. Specifically, the indoor unit 8200 includes a housing 8201, an air outlet 8202, the secondary battery 8203, and the like. Although FIG. 28 illustrates the case where the secondary battery 8203 is provided in the indoor unit 8200, the secondary battery 8203 may be provided in the outdoor unit 8204. Alternatively, the secondary batteries 8203 may be provided in both the indoor unit 8200 and the outdoor unit 8204. The air conditioner can be supplied with electric power from a commercial power supply and can use electric power stored in the secondary battery 8203. Particularly in the case where the secondary batteries 8203 are provided in both the indoor unit 8200 and the outdoor unit 8204, the air conditioner can be operated with the use of the secondary battery 8203 of one embodiment of the present invention as an uninterruptible power supply even when electric power cannot be supplied from a commercial power supply due to power failure or the like.

Note that although the split-type air conditioner including the indoor unit and the outdoor unit is illustrated in FIG. 28 as an example, the secondary battery of one embodiment of the present invention can be used in an air conditioner in which the function of an indoor unit and the function of an outdoor unit are integrated in one housing.

In FIG. 28, an electric refrigerator-freezer 8300 is an example of an electronic device including a secondary battery 8304 of one embodiment of the present invention. Specifically, the electric refrigerator-freezer 8300 includes a housing 8301, a refrigerator door 8302, a freezer door 8303, the secondary battery 8304, and the like. The secondary battery 8304 is provided in the housing 8301 in FIG. 28. The electric refrigerator-freezer 8300 can be supplied with electric power from a commercial power supply and can use electric power stored in the secondary battery 8304. Thus, the electric refrigerator-freezer 8300 can be operated with the use of the secondary battery 8304 of one embodiment of the present invention as an uninterruptible power supply even when electric power cannot be supplied from a commercial power supply due to power failure or the like.

Note that among the electronic devices described above, a high-frequency heating apparatus such as a microwave oven and an electronic device such as an electric rice cooker require high power in a short time. Therefore, the tripping of a breaker of a commercial power supply in use of the electronic device can be prevented by using the secondary battery of one embodiment of the present invention as an auxiliary power supply for supplying electric power which cannot be supplied enough by a commercial power supply.

In a time period when electronic devices are not used, particularly when the proportion of the amount of electric power which is actually used to the total amount of electric power which can be supplied from a commercial power supply source (such a proportion is referred to as a usage rate of electric power) is low, electric power is stored in the secondary battery, whereby an increase in the usage rate of electric power can be inhibited in a time period other than the above time period. For example, in the case of the electric refrigerator-freezer 8300, electric power is stored in the secondary battery 8304 in night time when the temperature is low and the refrigerator door 8302 and the freezer door 8303 are not opened or closed. Moreover, in daytime when the temperature is high and the refrigerator door 8302 and the freezer door 8303 are opened and closed, the usage rate of electric power in daytime can be kept low by using the secondary battery 8304 as an auxiliary power supply.

According to one embodiment of the present invention, the secondary battery can have excellent cycle performance and improved reliability. Furthermore, according to one embodiment of the present invention, a secondary battery with high charge and discharge capacity can be obtained; thus, the secondary battery itself can be made more compact and lightweight as a result of improved characteristics of the secondary battery. Thus, the secondary battery of one embodiment of the present invention is used in the electronic device described in this embodiment, whereby a more lightweight electronic device with a longer lifetime can be obtained.

This embodiment can be implemented in appropriate combination with the other embodiments.

### Embodiment 6

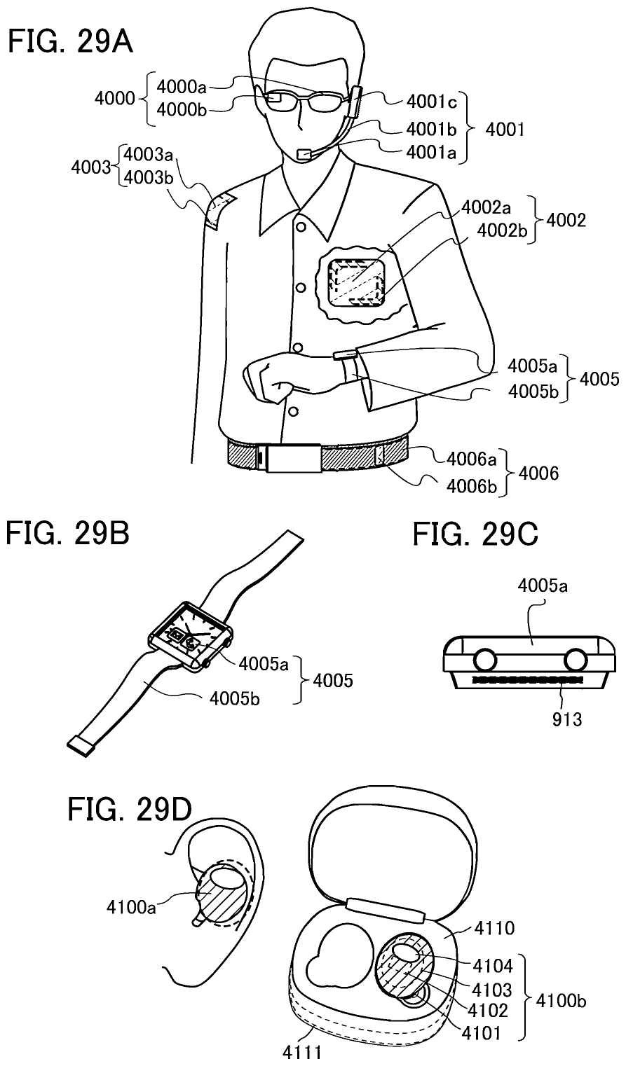

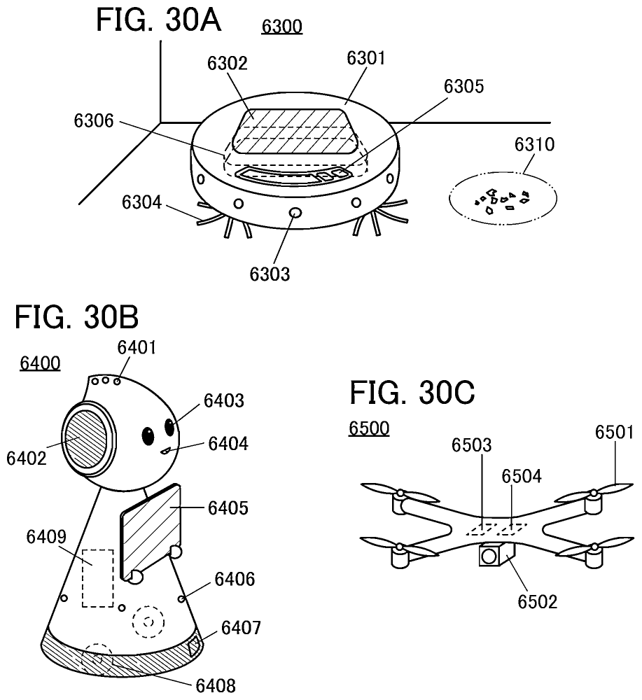

In this embodiment, examples of electronic devices each including the secondary battery described in the above embodiment are described with reference to FIG. 29A to FIG. 30C.

FIG. 29A illustrates examples of wearable devices. A secondary battery is used as a power source of a wearable device. To have improved splash resistance, water resistance, or dust resistance in daily use or outdoor use by a user, a wearable device is desirably capable of being charged with and without a wire whose connector portion for connection is exposed.

For example, the secondary battery of one embodiment of the present invention can be provided in a glasses-type device 4000 illustrated in FIG. 29A. The glasses-type device 4000 includes a frame 4000a and a display part 4000b. The secondary battery is provided in a temple of the frame 4000a having a curved shape, whereby the glasses-type device 4000 can be lightweight, can have a well-balanced weight, and can be used continuously for along time. With the use of the secondary battery of one embodiment of the present invention, space saving required with downsizing of a housing can be achieved.

The secondary battery of one embodiment of the present invention can be provided in a headset-type device 4001. The headset-type device 4001 includes at least a microphone part 4001a, a flexible pipe 4001b, and an earphone portion 4001c. The secondary battery can be provided in the flexible pipe 4001b or the earphone portion 4001c. With the use of the secondary battery of one embodiment of the present invention, space saving required with downsizing of a housing can be achieved.

The secondary battery of one embodiment of the present invention can be provided in a device 4002 that can be attached directly to a body. A secondary battery 4002b can be provided in a thin housing 4002a of the device 4002. With the use of the secondary battery of one embodiment of the present invention, space saving required with downsizing of a housing can be achieved.

The secondary battery of one embodiment of the present invention can be provided in a device 4003 that can be attached to clothes. A secondary battery 4003b can be provided in a thin housing 4003a of the device 4003. With the use of the secondary battery of one embodiment of the present invention, space saving required with downsizing of a housing can be achieved.

The secondary battery of one embodiment of the present invention can be provided in a belt-type device 4006. The belt-type device 4006 includes a belt portion 4006a and a wireless power feeding and receiving portion 4006b, and the secondary battery can be provided inside the belt portion 4006a. With the use of the secondary battery of one embodiment of the present invention, space saving required with downsizing of a housing can be achieved.

The secondary battery of one embodiment of the present invention can be provided in a watch-type device 4005. The watch-type device 4005 includes a display portion 4005a and a belt portion 4005b, and the secondary battery can be provided in the display portion 4005a or the belt portion 4005b. With the use of the secondary battery of one embodiment of the present invention, space saving required with downsizing of a housing can be achieved.

The display portion 4005a can display various kinds of information such as time and reception information of an e-mail or an incoming call.

In addition, the watch-type device 4005 is a wearable device that is wound around an arm directly; thus, a sensor that measures the pulse, the blood pressure, or the like of the user may be incorporated therein. Data on the exercise quantity and health of the user can be stored to be used for health maintenance.

FIG. 29B is a perspective view of the watch-type device 4005 that is detached from an arm.

FIG. 29C is a side view. FIG. 29C illustrates a state where the secondary battery 913 is incorporated in the watch-type device 4005. The secondary battery 913 is the secondary battery described in Embodiment 4. The secondary battery 913, which is small and lightweight, overlaps with the display portion 4005a.

FIG. 29D illustrates an example of wireless earphones. The wireless earphones shown here consist of, but are not limited to, a pair of main bodies 4100a and 4100b.

Each of the main bodies 4100a and 4100b includes a driver unit 4101, an antenna 4102, and a secondary battery 4103. Each of the main bodies 4100a and 4100b may also include a display portion 4104. Moreover, each of the main bodies 4100a and 4100b preferably includes a substrate where a circuit such as a wireless IC is provided, a terminal for charge, and the like. Each of the main bodies 4100a and 4100b may also include a microphone.

A case 4110 includes a secondary battery 4111. Moreover, the case 4110 preferably includes a substrate where a circuit such as a wireless IC or a charge control IC is provided, and a terminal for charge. The case 4110 may also include a display portion, a button, and the like.

The main bodies 4100a and 4100b can communicate wirelessly with another electronic device such as a smartphone. Thus, sound data and the like transmitted from another electronic device can be played through the main bodies 4100a and 4100b. When the main bodies 4100a and 4100b include a microphone, sound captured by the microphone is transmitted to another electronic device, and sound data obtained by processing with the electronic device can be transmitted to and played through the main bodies 4100a and 4100b. Hence, the wireless earphones can be used as a translator, for example.

The secondary battery 4103 included in the main body 4100a can be charged by the secondary battery 4111 included in the case 4100. As the secondary battery 4111 and the secondary battery 4103, the coin-type secondary battery or the cylindrical secondary battery of the foregoing embodiment, for example, can be used. A secondary battery whose positive electrode includes the positive electrode active material 100 obtained in Embodiment 1 has a high energy density; thus, with the use of the secondary battery as the secondary battery 4103 and the secondary battery 4111, space saving required with downsizing of the wireless earphones can be achieved.

FIG. 30A illustrates an example of a cleaning robot. A cleaning robot 6300 includes a display portion 6302 placed on the top surface of a housing 6301, a plurality of cameras 6303 placed on the side surface of the housing 6301, a brush 6304, operation buttons 6305, a secondary battery 6306, a variety of sensors, and the like. Although not illustrated, the cleaning robot 6300 is provided with a tire, an inlet, and the like. The cleaning robot 6300 is self-propelled, detects dust 6310, and sucks up the dust through the inlet provided on the bottom surface.

For example, the cleaning robot 6300 can determine whether there is an obstacle such as a wall, furniture, or a step by analyzing images taken by the cameras 6303. In the case where the cleaning robot 6300 detects an object, such as a wire, that is likely to be caught in the brush 6304 by image analysis, the rotation of the brush 6304 can be stopped. The cleaning robot 6300 further includes a secondary battery 6306 of one embodiment of the present invention and a semiconductor device or an electronic component. The cleaning robot 6300 including the secondary battery 6306 of one embodiment of the present invention can be a highly reliable electronic device that can operate for a long time.

FIG. 30B illustrates an example of a robot. A robot 6400 illustrated in FIG. 30B includes a secondary battery 6409, an illuminance sensor 6401, a microphone 6402, an upper camera 6403, a speaker 6404, a display portion 6405, a lower camera 6406, an obstacle sensor 6407, a moving mechanism 6408, an arithmetic device, and the like.

The microphone 6402 has a function of detecting a speaking voice of a user, an environmental sound, and the like. The speaker 6404 has a function of outputting sound. The robot 6400 can communicate with a user using the microphone 6402 and the speaker 6404.

The display portion 6405 has a function of displaying various kinds of information. The robot 6400 can display information desired by a user on the display portion 6405. The display portion 6405 may be provided with a touch panel. Moreover, the display portion 6405 may be a detachable information terminal, in which case charge and data communication can be performed when the display portion 6405 is set at the home position of the robot 6400.

The upper camera 6403 and the lower camera 6406 each have a function of taking an image of the surroundings of the robot 6400. The obstacle sensor 6407 can detect an obstacle in the direction where the robot 6400 advances with the moving mechanism 6408. The robot 6400 can move safely by recognizing the surroundings with the upper camera 6403, the lower camera 6406, and the obstacle sensor 6407.

The robot 6400 further includes the secondary battery 6409 of one embodiment of the present invention and a semiconductor device or an electronic component. The robot 6400 including the secondary battery of one embodiment of the present invention can be a highly reliable electronic device that can operate for a long time.

FIG. 30C illustrates an example of a flying object. A flying object 6500 illustrated in FIG. 30C includes propellers 6501, a camera 6502, a secondary battery 6503, and the like and has a function of flying autonomously.

For example, image data taken by the camera 6502 is stored in an electronic component 6504. The electronic component 6504 can analyze the image data to detect whether there is an obstacle in the way of the movement. Moreover, the electronic component 6504 can estimate the remaining battery level from a change in the power storage capacity of the secondary battery 6503. The flying object 6500 further includes the secondary battery 6503 of one embodiment of the present invention. The flying object 6500 including the secondary battery of one embodiment of the present invention can be a highly reliable electronic device that can operate for a long time.

This embodiment can be implemented in appropriate combination with the other embodiments.

### Embodiment 7

In this embodiment, examples of vehicles each including the secondary battery of one embodiment of the present invention will be described.

The use of secondary batteries in vehicles enables production of next-generation clean energy vehicles such as hybrid electric vehicles (HVs), electric vehicles (EVs), and plug-in hybrid electric vehicles (PHVs).

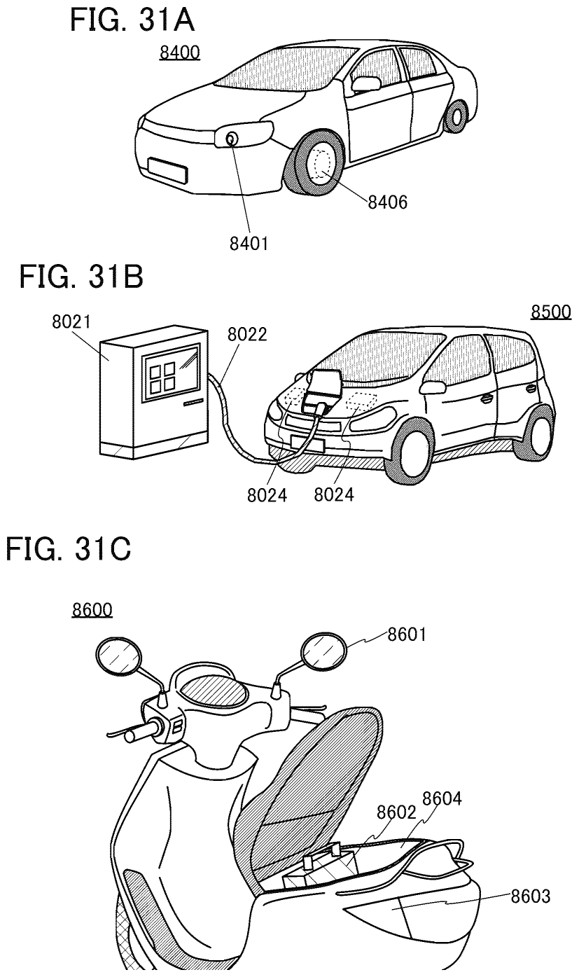

FIG. 31 illustrates examples of a vehicle including the secondary battery of one embodiment of the present invention. An automobile 8400 illustrated in FIG. 31A is an electric vehicle that runs on the power of an electric motor. Alternatively, the automobile 8400 is a hybrid electric vehicle capable of driving using either an electric motor or an engine as appropriate. The use of one embodiment of the present invention achieves a high-mileage vehicle. The automobile 8400 includes the secondary battery. As the secondary battery, the modules of the secondary batteries illustrated in FIG. 17C and FIG. 17D may be arranged to be used in a floor portion in the automobile. Alternatively, a battery pack in which a plurality of secondary batteries illustrated in FIG. 20 are combined may be placed in the floor portion in the automobile. The secondary battery can be used not only for driving an electric motor 8406, but also for supplying electric power to a light-emitting device such as a headlight 8401 or a room light (not shown).

The secondary battery can also supply electric power to a display device included in the automobile 8400, such as a speedometer or a tachometer. Furthermore, the secondary battery can supply electric power to a semiconductor device included in the automobile 8400, such as a navigation system.

An automobile 8500 illustrated in FIG. 31B can be charged when the secondary battery included in the automobile 8500 is supplied with electric power through external charge equipment by a plug-in system, a contactless power feeding system, and/or the like. FIG. 31B illustrates a state where a secondary battery 8024 included in the automobile 8500 is charged with the use of a ground-based charging apparatus 8021 through a cable 8022. Charge can be performed as appropriate by a given method such as CHAdeMO (registered trademark) or Combined Charging System as a charging method, the standard of a connector, or the like. The charging apparatus 8021 may be a charge station provided in a commerce facility or a power supply in a house. For example, with the use of a plug-in technique, the secondary battery 8024 included in the automobile 8500 can be charged by being supplied with electric power from outside. The charge can be performed by converting AC electric power into DC electric power through a converter such as an ACDC converter.

Although not shown, the vehicle may include a power receiving device so that it can be charged by being supplied with electric power from an above-ground power transmitting device in a contactless manner. In the case of the contactless power feeding system, by fitting a power transmitting device in a road or an exterior wall, charge can be performed not only when the vehicle is stopped but also when driven. In addition, the contactless power feeding system may be utilized to perform transmission and reception of electric power between vehicles. Furthermore, a solar cell may be provided in the exterior of the vehicle to charge the secondary battery when the vehicle stops or moves. To supply electric power in such a contactless manner, an electromagnetic induction method or a magnetic resonance method can be used.

FIG. 31C illustrates an example of a motorcycle including the secondary battery of one embodiment of the present invention. A motor scooter 8600 illustrated in FIG. 31C includes a secondary battery 8602, side mirrors 8601, and direction indicators 8603. The secondary battery 8602 can supply electric power to the direction indicators 8603.

In the motor scooter 8600 illustrated in FIG. 31C, the secondary battery 8602 can be held in an under-seat storage 8604. The secondary battery 8602 can be held in the under-seat storage 8604 even when the under-seat storage 8604 is small. The secondary battery 8602 is detachable; thus, the secondary battery 8602 is carried indoors when charged, and is stored before the motor scooter is driven.

According to one embodiment of the present invention, the secondary battery can have improved cycle performance and the charge and discharge capacity of the secondary battery can be increased. Thus, the secondary battery itself can be made more compact and lightweight. The compact and lightweight secondary battery contributes to a reduction in the weight of a vehicle, and thus increases the mileage. Furthermore, the secondary battery included in the vehicle can be used as a power source for supplying electric power to products other than the vehicle. In such a case, the use of a commercial power supply can be avoided at peak time of electric power demand, for example. Avoiding the use of a commercial power supply at peak time of electric power demand can contribute to energy saving and a reduction in carbon dioxide emissions. Moreover, the secondary battery with excellent cycle performance can be used over a long period; thus, the use amount of rare metals typified by cobalt can be reduced.

This embodiment can be implemented in appropriate combination with the other embodiments.

### Example 1

In this example, an ionic liquid of one embodiment of the present invention, 1-methyl-3-(2,2,2-trifluoroethyl)-imidazolium bis(fluorosulfonyl)imide (abbreviation: F3EMI-FSI), which includes a cation represented by Structural Formula (100) and an anion represented by Structural Formula (200), was synthesized and the characteristics thereof were evaluated. The structural formula of F3EMI-FSI is shown below.

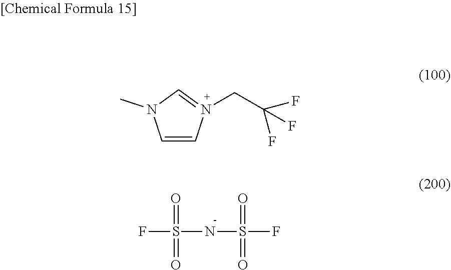

### Synthesis Method

### Step 1: Synthesis of F3EMI-TfO

First, 50.2 g (216 mmol) of 2,2,2-trifluoroethyltriflate was added to a 300-mL three-neck flask. Then, 9.70 g (118 mmol) of 1-methylimidazole and 50.0 mL of acetonitrile were dropped into the three-neck flask that was being cooled in an ice bath. After that, stirring was performed under reflux at 90° C. for 6 hours. This mixture was concentrated to give 37.4 g of a target pale yellow liquid (F3EMI-TfO) (yield: 100%). The synthesis scheme in Step 1 is shown in Formula (a-1) below.

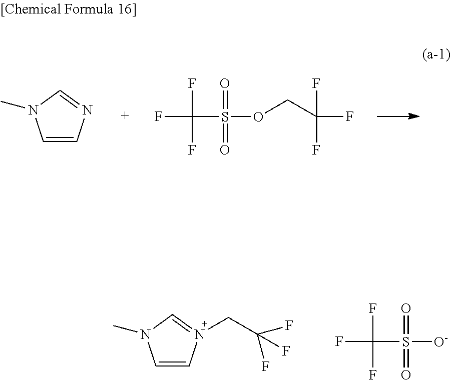

### Step 2: Synthesis of F3EMI-FSI

F3EMI-TfO obtained in Step 1 was dissolved in 58.6 mL of water, 26.1 g (119 mmol) of potassium bis(fluorosulfonyl)imide was added thereto while stirring was performed at room temperature, and the mixture was stirred for 15.5 hours. After that, the mixture was extracted with dichloromethane and water and separated into an organic layer and water; then, the organic layer was dried with magnesium sulfate. This mixture was subjected to gravity filtration to remove magnesium sulfate, and then the organic layer was concentrated to give 11.6 g of a target pale yellow liquid. In order to obtain a target substance contained in an aqueous layer, ethyl acetate was added to the aqueous layer to extract an organic layer. After the obtained organic layer was dried with magnesium sulfate, the magnesium sulfate was removed from the mixture by gravity filtration to give a target pale yellow liquid. This pale yellow liquid was combined with the pale yellow liquid obtained above, and concentrated and dried to give 21.7 g of a pale yellow liquid (yield: 53.0%). The synthesis scheme in Step 1 is shown in Formula (a-2) below.

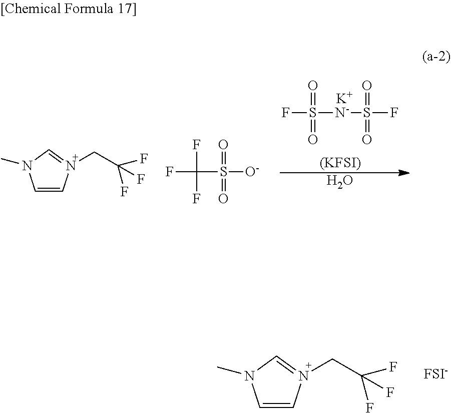

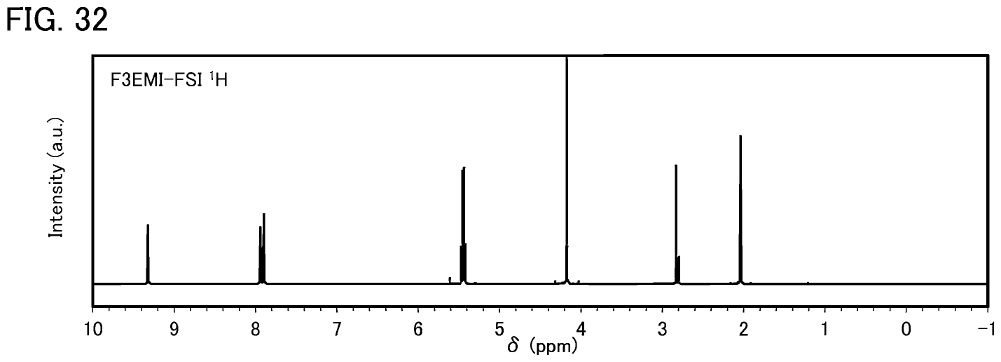

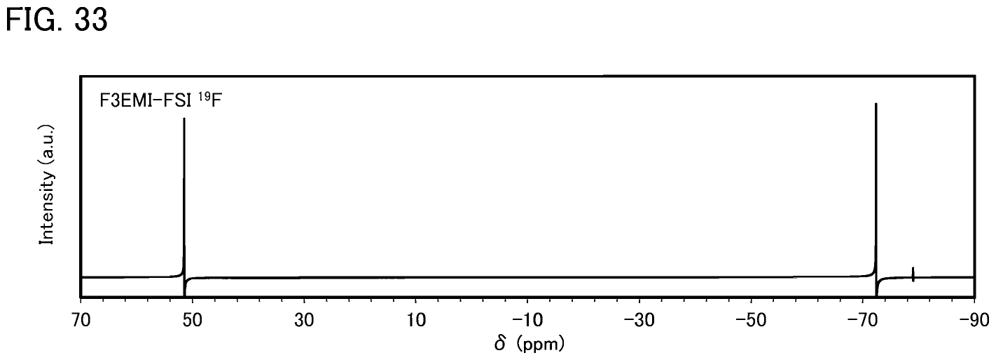

The pale yellow liquid obtained above was analyzed by nuclear magnetic resonance spectroscopy (¹H-NMR and ¹⁹F-NMR), and the results are shown below. FIG. 32 shows a ¹H-NMR chart. FIG. 33 is a ¹⁹F-NMR chart. These results indicate that F3EMI-FSI was obtained by the aforementioned synthesis method.

¹H NMR (CD₃COCD₃-d6, 500 MHz): δ=4.18 (s, 3H), 5.45 (q, J=8.5 Hz, 2H), 7.90 (t, J=1.5 Hz, 1H), 7.94 (s, 2H), 9.32 (s, 1H).

¹⁹F NMR (CD₃COCD₃-d6, 500 MHz): δ=−72.4 (s, 3F), 54.4 (s, 2F).

### <CV>

Next, the oxidation resistance of F3EMI-FSI synthesized above was evaluated by cyclic voltammetry (CV).

First, 2.15 mol/L of LiFSI was dissolved as a lithium salt in F3EMI-FSI, which is the ionic liquid of one embodiment of the present invention, so that an electrolyte was formed.

As an ionic liquid of a comparative example, 1-ethyl-3-methylimidazolium bis(fluorosulfonyl)imide (abbreviation: EMI-FSI) was prepared. EMI, which is the cation of the ionic liquid, does not contain fluorine at a terminal. As a lithium salt, 2.15 mol/L of LiFSI was dissolved in EMI-FSI, so that an electrolyte was formed.

The CV conditions were as follows. As a working electrode, a mixture of AB and PVdF with a weight ratio of 1:1 was applied to aluminum foil coated with carbon. The working electrode has a diameter of 12 mm and an area of 1.1304 cm². Lithium was used for a counter electrode. As a separator, polypropylene and glass fiber filter paper (produced by Whatman Ltd.) were stacked with the polypropylene positioned on the working electrode side. An aluminum-clad material was used for a positive electrode can. The scanning rate was 0.5 mV·s⁻¹. The measurement temperature was 25° C. Scanning was performed five times. The voltage was in the range of 2.0 to 5.0 V.

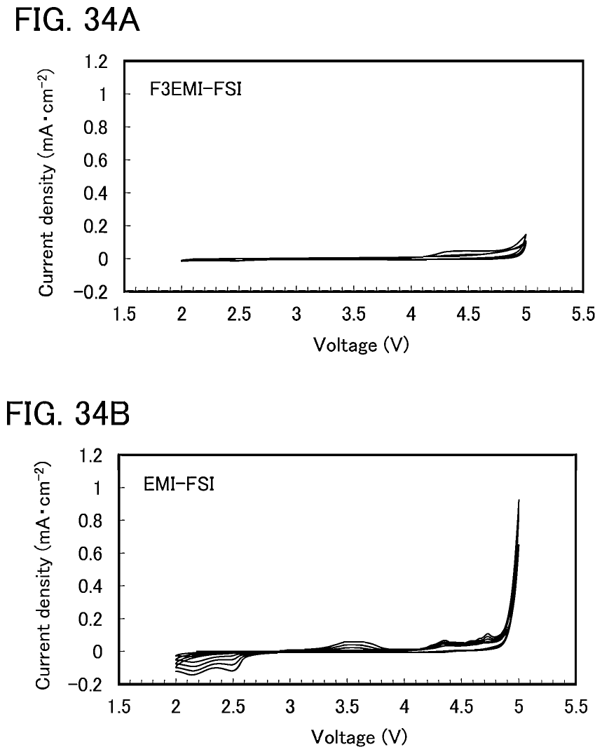

FIG. 34A shows a cyclic voltammogram of the electrolyte including F3EMI-FSI, which is the ionic liquid of one embodiment of the present invention. FIG. 34B shows a cyclic voltammogram of the electrolyte including EMI-FSI, which is the ionic liquid of a comparative example.

As shown in FIG. 34A, the electrolyte including F3EMI-FSI, which contains fluorine at a terminal of a cation, has no peak observed at a voltage of 4.7 V or lower, that is, the electrolyte was not oxidized. In contrast, peaks were observed at around 4.4 V and around 4.7 V in the electrolyte including EMI-FSI as shown in FIG. 34B, which suggested that the electrolyte was oxidized.

It was found from the above that the terminal of the cation substituted by fluorine contributed to an improvement in the oxidation resistance of the ionic liquid.

### <Charge and Discharge Characteristics>

Next, a secondary battery was fabricated using F3EMI-FSI, which is the ionic liquid of one embodiment of the present invention, and the charge and discharge characteristics thereof were evaluated.

As an electrolyte, 2.15 mol/L of LiFSI was dissolved as a lithium salt in F3EMI-FSI.

A positive electrode active material included in a positive electrode was formed by the method described in Embodiment 2 except that heating in Step S15 was not performed and an added element X source was mixed in two steps and heated. The positive electrode active material formed in this example is described with reference to FIG. 14.

As in Step S14 in FIG. 14, commercially available lithium cobalt oxide (Cellseed C-10N produced by NIPPON CHEMICAL INDUSTRIAL CO., LTD.), which contains cobalt as a transition metal M and does not contain any added element, was prepared as LiMO₂.

Heating in Step S15 was not performed.

As the X source in Step S20, lithium fluoride and magnesium fluoride were mixed at a molar ratio of LiF:MgF₂=1:3.

As Step S31, lithium fluoride and magnesium fluoride were mixed so that 1 atomic % of magnesium was contained in lithium cobalt oxide.

As Step S33, the mixture was heated at 900° C. for 20 hours in a muffle furnace. At this time, a container to which the mixture was put was covered with a lid. After the muffle furnace was filled with an oxygen atmosphere, no flowing was performed.

As an added element X in the second mixing, nickel hydroxide and aluminum hydroxide were prepared. Nickel hydroxide and aluminum hydroxide were mixed so that 0.5 atomic % of nickel and 0.5 atomic % of aluminum were contained in lithium cobalt oxide to which magnesium and fluorine were added.

As heating after the second mixing of the added element X, the mixture was heated at 850° C. for 10 hours in a muffle furnace. Also in that case, a container to which the mixture was put was covered with a lid. The atmosphere in the muffle furnace was an oxygen atmosphere with an oxygen flow rate of 10 L/min. Then, the mixture was cooled to room temperature and heated again at 850° C. for 10 hours to be used as the positive electrode active material.

Acetylene black (AB) was prepared as a conductive material, and polyvinylidene fluoride (PVDF) was prepared as a binder. A slurry was formed by mixing the positive electrode active material, AB, and PVDF at a weight ratio of 95:3:2, and the slurry was applied to a current collector of aluminum. As a solvent of the slurry, NMP was used.

After the slurry was applied to the current collector, the solvent was volatilized. Then, pressure was applied at 120 kN/m with a calender roll. Through the above steps, the positive electrode was obtained. The loading level of the positive electrode active material was approximately 10 mg/cm².

A lithium metal was prepared for a counter electrode.

Three porous polyimide films (manufactured by Tokyo Ohka Kogyo Co., Ltd.) were stacked to be used as a separator.

With a laminate film used as an exterior body, a half cell including the electrolyte, the positive electrode, and the like was formed.

The secondary battery fabricated above was subjected to charge and discharge tests. CC/CV charge (0.2 C, 4.6 V, 0.02 C cut) and CC discharge (0.2 C, 2.5 V cut) were performed, and a 10-minute break was taken before the next charge. The measurement temperature was set to 25° C. Note that in this example and the like, 1 C was 200 mA/g.

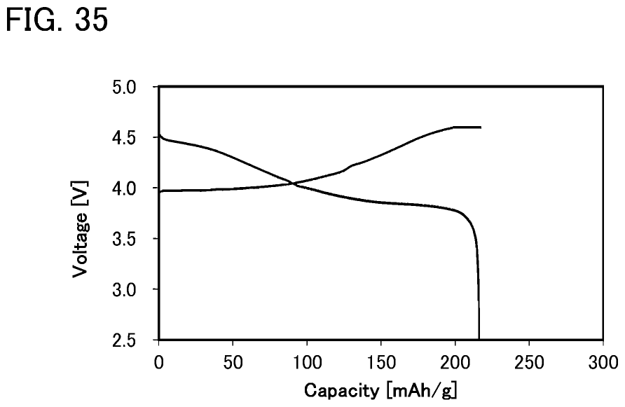

FIG. 35 shows the charge and discharge curves in the second cycle offering stable charge and discharge capacity. The discharge capacity in the second cycle was 216.4 mAh/g. FIG. 35 shows that the secondary battery including the ionic liquid of one embodiment of the present invention has favorable charge and discharge characteristics.

### Example 2

In this example, an ionic liquid of one embodiment of the present invention, 1-(2,2-difluoroethyl)-3-methyl-imidazolium bis(fluorosulfonyl)imide (abbreviation: F2EMI-FSI), which includes a cation represented by Structural Formula (150) and an anion represented by Structural Formula (200), was synthesized and the characteristics thereof were evaluated. The structural formula of F2EMI-FSI is shown below.

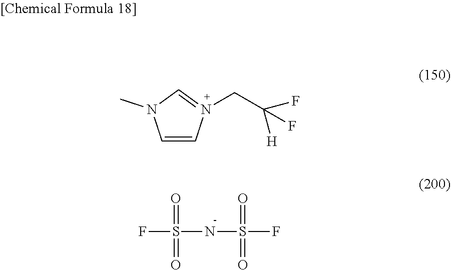

### Synthesis Method

### Step 1: Synthesis of 1-(2,2-difluoroethyl)-3-methyl-imidazolium triflate (Abbreviation: F2EMI-TfO)

First, 46.24 g (216.0 mmol) of trifluoromethanesulfonate 2,2-difluoroethyl was put into a 300-mL three-neck flask, and while the three-neck flask was soaked in an ice bath, a mixture of 13.20 g (160.8 mmol) of 1-methylimidazole and 66.4 mL of acetonitrile was dropped thereto from a dropping funnel for approximately 25 minutes while being stirred. After that, the mixture was heated and refluxed in a nitrogen atmosphere for 3 hours in an oil bath set to 90° C.; then, the reaction solution was dried to give 50.68 g of a yellow liquid (crude yield: 106.4%). Furthermore, the yellow liquid was washed and extracted with water and hexane; then, an aqueous layer was concentrated to give 49.28 g of a yellow liquid (yield: 103.5%). As a result of measurements of the yellow liquid by ¹H-NMR and ¹⁹F-NMR (solvent: heavy acetone), a peak probably derived from the target substance was obtained. The synthesis scheme of F2EMI-TfO in Step 1 is shown in Formula (b-1) below.

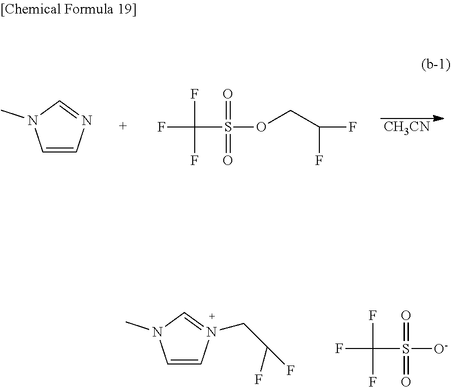

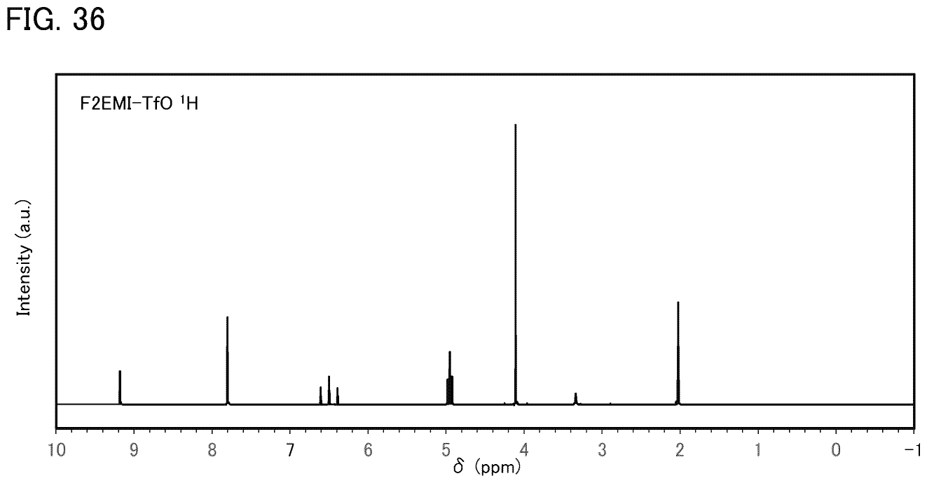

The yellow liquid obtained in Step 1 above was analyzed by nuclear magnetic resonance spectroscopy (¹H-NMR and ¹⁹F-NMR), and the results are shown below. FIG. 36 shows a ¹H-NMR chart. These results indicate that F2EMI-TfO was synthesized in this synthesis example.

¹H-NMR (CD₃COCD₃, 500 MHz): δ=4.12 (s, 3H), 4.97 (t, J=15.5 Hz, 2H), 6.51 (t, J=54.0 Hz, 1H), 7.82 (s, 2H), 9.20 (s, 1H).

¹⁹F-NMR (CD₃COCD₃, 500 MHz): δ=−125.24 (s, 2F), −78.81 (s, 3F).

### Step 2: Synthesis of 1-(2,2-difluoroethyl)-3-methyl-imidazolium bis(fluorosulfonyl)imide (Abbreviation: F2EMI-FSI)

To F2EMI-TfO obtained in Step 1 was added 77.1 mL of water and 36.33 g (165.7 mmol) of potassium bis(fluorosulfonyl)imide, and stirring was performed at room temperature for 25 hours. After that, the mixture was extracted with ethyl acetate three times; then, an organic layer was washed with water three times. The organic layer was dried with magnesium sulfate and subjected to suction filtration. The filtrate was concentrated and dried, and a precipitate was separated by filtration with a membrane filter to give 46.32 g of a yellow liquid (crude yield: 88.0%).

Dichloromethane and a small amount of water were added to the yellow liquid and washed in a separating funnel; then, an organic layer was fractionated and a small amount of water was added thereto again for washing. After the organic layer and an aqueous layer were separated, magnesium sulfate and activated carbon were added to the organic layer and stirring was performed; then, magnesium sulfate and activated carbon were removed by suction filtration through Celite. The obtained filtrate was dried to give 23.38 g of a target yellow transparent liquid (yield: 44.4%). As a result of measurements of the liquid by ¹H-NMR and ¹⁹F-NMR (solvent: heavy acetone), a peak derived from an impurity that was found in the crude product disappeared and a peak derived from the target substance was obtained. The synthesis scheme of F2EMI-FSI in Step 2 is shown in Formula (b-2) below.

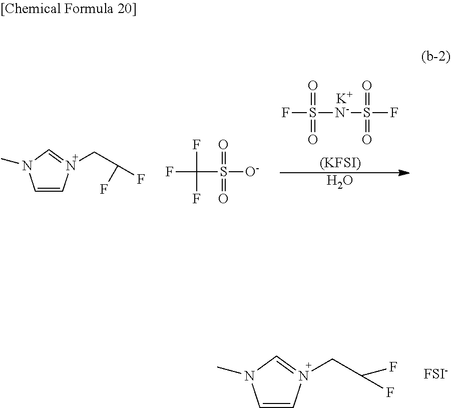

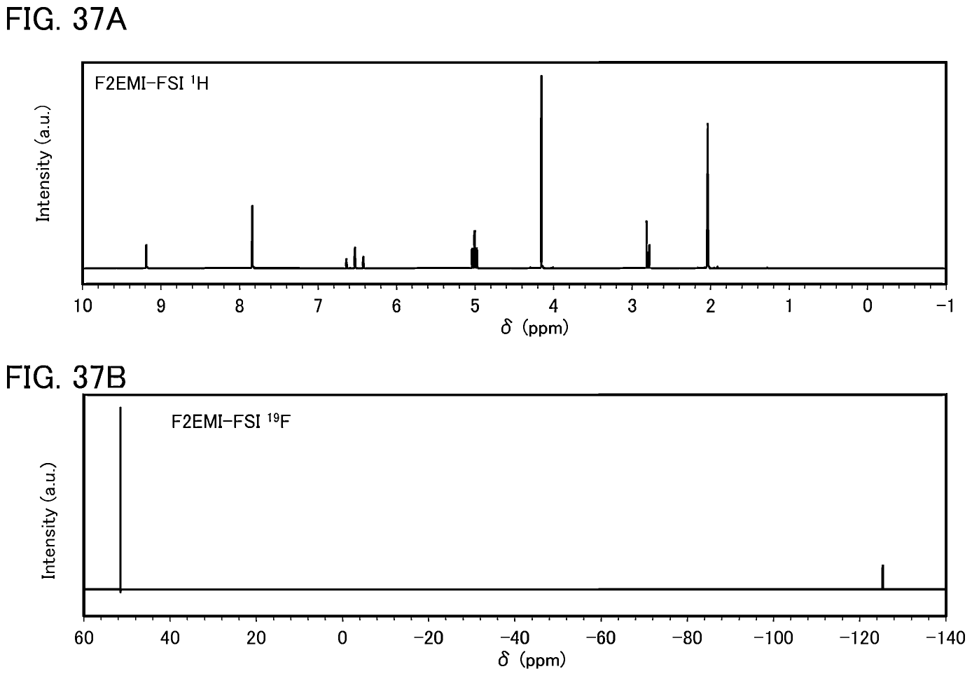

The yellow liquid obtained in Step 2 above was analyzed by nuclear magnetic resonance spectroscopy (¹H-NMR and ¹⁹F-NMR), and the results are shown below. FIG. 37A shows a ¹H-NMR chart. FIG. 37B shows a ¹⁹F-NMR chart. These results indicate that F2EMI-FSI was synthesized in this synthesis example.

¹H-NMR (CD₃COCD₃, 500 MHz): δ=4.14 (s, 3H), 4.98 (t, J=15.0 Hz, 2H), 6.52 (t, J=54.0 Hz, 1H), 7.82 (s, 2H), 9.16 (s, 1H).

¹⁹F-NMR (CD₃COCD₃, 500 MHz): δ=−125.22 (s, 2F), 51.43 (s, 2F).

Next, the oxidation resistance of F2EMI-FSI synthesized above was evaluated by cyclic voltammetry (CV).

First, 2.15 mol/L of LiFSI was dissolved as a lithium salt in F2EMI-FSI, which is the ionic liquid of one embodiment of the present invention, so that an electrolyte was formed.

As an ionic liquid of a comparative example, 1-ethyl-3-methylimidazolium bis(fluorosulfonyl)imide (abbreviation: EMI-FSI) was prepared. EMI, which is the cation of the ionic liquid, does not contain fluorine at a terminal. As a lithium salt, 2.15 mol/L of LiFSI was dissolved in EMI-FSI, so that an electrolyte was formed.

The CV conditions were as follows. As a working electrode, a mixture of AB and PVdF with a weight ratio of 1:1 was applied to aluminum foil coated with carbon. The working electrode has a diameter of 12 mm and an area of 1.1304 cm². Lithium was used for a counter electrode. As a separator, polypropylene and glass fiber filter paper (produced by Whatman Ltd.) were stacked with the polypropylene positioned on the working electrode side. An aluminum-clad material was used for a positive electrode can. The scanning rate was 0.5 mV·s⁻¹. The measurement temperature was 25° C. Scanning was performed five times. The voltage was in the range of 2.0 to 5.0 V.

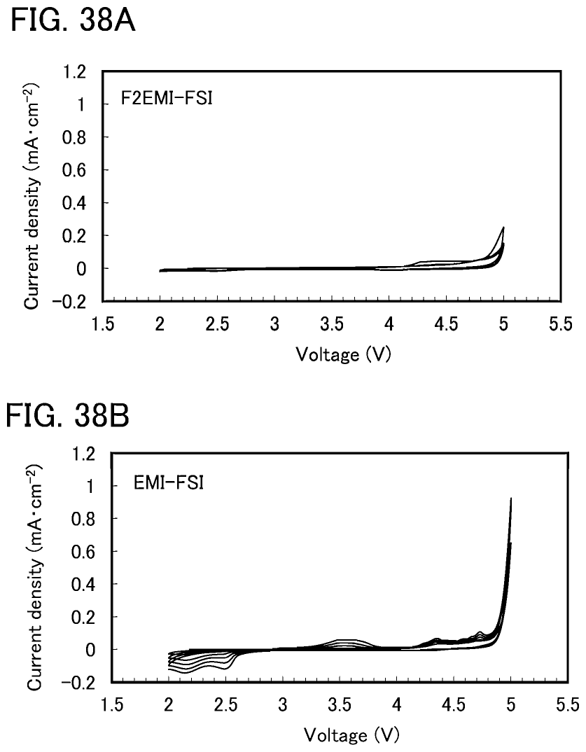

FIG. 38A shows a cyclic voltammogram of the electrolyte including F2EMI-FSI, which is the ionic liquid of one embodiment of the present invention. FIG. 38B shows a cyclic voltammogram of the electrolyte including EMI-FSI, which is the ionic liquid of a comparative example.

As shown in FIG. 38A, the electrolyte including F2EMI-FSI, which contains fluorine at a terminal of a cation, has no peak observed at a voltage of 4. 7 V or lower, that is, the electrolyte was not oxidized. In contrast, peaks were observed at around 4.4 V and around 4.7 V in the electrolyte including EMI-FSI as shown in FIG. 38B, which suggested that the electrolyte was oxidized.

It was found from the above that the terminal of the cation substituted by fluorine contributed to an improvement in the oxidation resistance of the ionic liquid.

### <Charge and Discharge Characteristics>

Next, a secondary battery was fabricated using F2EMI-FSI, which is the ionic liquid of one embodiment of the present invention, and the charge and discharge characteristics thereof were evaluated.

As an electrolyte, 2.15 mol/L of LiFSA was dissolved as a lithium salt in F2EMI-FSI.

A positive electrode active material included in a positive electrode was formed by the method described in Embodiment 2 except that heating in Step S15 was not performed and an added element X source was mixed in two steps and heated. The positive electrode active material formed in this example is described with reference to FIG. 14.

As in Step S14 in FIG. 14, commercially available lithium cobalt oxide (Cellseed C-10N produced by NIPPON CHEMICAL INDUSTRIAL CO., LTD.), which contains cobalt as a transition metal M and does not contain any added element, was prepared as LiMO₂.

Heating in Step S15 was not performed.

As the X source in Step S20, lithium fluoride and magnesium fluoride were mixed at a molar ratio of LiF:MgF₂=1:3.

As Step S31, lithium fluoride and magnesium fluoride were mixed so that 1 atomic % of magnesium was contained in lithium cobalt oxide.

As Step S33, the mixture was heated at 900° C. for 20 hours in a muffle furnace. At this time, a container to which the mixture was put was covered with a lid. After the muffle furnace was filled with an oxygen atmosphere, no flowing was performed.

As an added element X in the second mixing, nickel hydroxide and aluminum hydroxide were prepared. Nickel hydroxide and aluminum hydroxide were mixed so that 0.5 atomic % of nickel and 0.5 atomic % of aluminum were contained in lithium cobalt oxide to which magnesium and fluorine were added.

As heating after the second mixing of the added element X, the mixture was heated at 850° C. for 10 hours in a muffle furnace. Also in that case, a container to which the mixture was put was covered with a lid. The atmosphere in the muffle furnace was an oxygen atmosphere with an oxygen flow rate of 10 L/min. Then, the mixture was cooled to room temperature and heated again at 850° C. for 10 hours to be used as the positive electrode active material.

Acetylene black (AB) was prepared as a conductive material, and polyvinylidene fluoride (PVDF) was prepared as a binder. A slurry was formed by mixing the positive electrode active material, AB, and PVDF at a weight ratio of 95:3:2, and the slurry was applied to a current collector of aluminum. As a solvent of the slurry, NMP was used.

After the slurry was applied to the current collector, the solvent was volatilized. Then, pressure was applied at 120 kN/m with a calender roll. Through the above steps, the positive electrode was obtained. The loading level of the positive electrode active material was approximately 10 mg/cm².

A lithium metal was prepared for a counter electrode.

Three porous polyimide films (manufactured by Tokyo Ohka Kogyo Co., Ltd.) were stacked to be used as a separator.

With a laminate film used as an exterior body, a half cell including the electrolyte, the positive electrode, and the like was formed.

The secondary battery fabricated above was subjected to charge and discharge tests. CC/CV charge (0.2 C, 4.6 V, 0.02 C cut) and CC discharge (0.2 C, 2.5 V cut) were performed, and a 10-minute break was taken before the next charge. The measurement temperature was set to 45° C. Note that in this example and the like, 1 C was 200 mA/g.

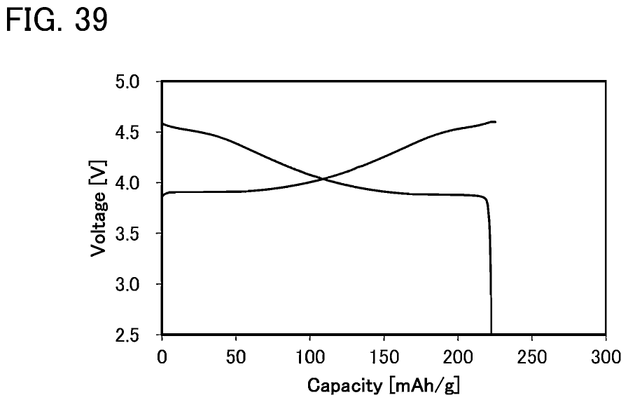

FIG. 39 shows the charge and discharge curves in the second cycle offering stable charge and discharge capacity. The discharge capacity in the second cycle was 227.7 mAh/g. FIG. 39 shows that the secondary battery including the ionic liquid of one embodiment of the present invention has favorable charge and discharge characteristics.

### REFERENCE NUMERALS

100: positive electrode active material, 100a: surface portion, 100b: inner portion, 101: crystal grain boundary, 102: filling portion, 103: projection, 104: coating film, 200: active material layer, 201: graphene compound, 300: secondary battery, 301: positive electrode can, 302: negative electrode can, 303: gasket, 304: positive electrode, 305: positive electrode current collector, 306: positive electrode active material layer, 307: negative electrode, 308: negative electrode current collector, 309: negative electrode active material layer, 310: separator, 500: secondary battery, 501: positive electrode current collector, 502: positive electrode active material layer, 503: positive electrode, 504: negative electrode current collector, 505: negative electrode active material layer, 506: negative electrode, 507: separator, 508: electrolyte, 509: exterior body, 510: positive electrode lead electrode, 511: negative electrode lead electrode, 512: stack, 514: region

1. An ionic liquid comprising a cation represented by General Formula (G1) and an anion represented by Structural Formula (200), 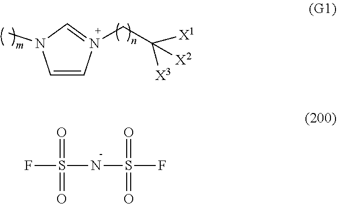
wherein in the formula, X¹ to X³ each independently represent any one of fluorine, chlorine, bromine, iodine, and hydrogen,
wherein at least two of X¹ to X³ each independently represent any one of the fluorine, the chlorine, the bromine, and the iodine, and
wherein n and m each independently represent 0 to 5.

2. An ionic liquid comprising a cation represented by Structural Formula (100) and an anion represented by Structural Formula (200),

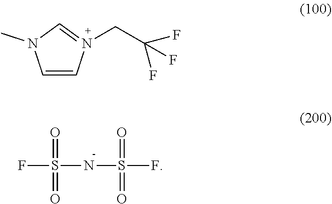

3. An ionic liquid comprising a cation represented by Structural Formula (150) and an anion represented by Structural Formula (200),

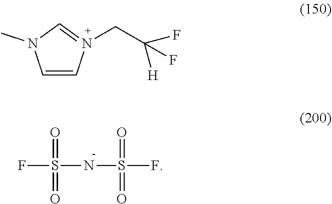

4. A secondary battery comprising a positive electrode, a negative electrode, and an electrolyte,
wherein the electrolyte comprises the ionic liquid according to claim 1.

5. The secondary battery according to claim 4,
wherein the electrolyte further comprises an additive agent, and
wherein the additive agent is at least one of succinonitrile, adiponitrile, fluoroethylene carbonate, and propane sultone.

6. The secondary battery according to claim 4,
wherein the positive electrode comprises a positive electrode active material,
wherein the positive electrode active material comprises lithium cobalt oxide to which magnesium, fluorine, aluminum, and nickel are added, and
wherein an XRD pattern at least has a diffraction peak at 2θ=19.30±0.20° and 2θ=45.55±0.10° when the positive electrode is analyzed by powder X-ray diffraction with CuKα₁ radiation in an argon atmosphere in the following manner: the positive electrode and a counter electrode of a lithium metal are used, a mixture in which 2 wt % of vinylene carbonate is added to lithium hexafluorophosphate, ethylene carbonate, and diethyl carbonate is used as the electrolyte, constant current charge with a current value of 0.5 C (note that 1 C=137 mA/g) is performed to a voltage of 4.6 V in an environment at 25° C., and then constant voltage charge is performed until the current value becomes 0.01 C.

7. The secondary battery according to claim 6,
wherein a diffusion state of each of the magnesium and the aluminum included in the positive electrode active material varies on each crystal plane of a surface portion.

8. The secondary battery according to claim 7,
wherein the positive electrode active material has a crystal structure belonging to a space group R-3m, and
wherein the magnesium and the aluminum exist in a deeper position of the surface portion in a region with a crystal plane other than (001) than in a region with the crystal plane (001).

9. An electronic device comprising:
the secondary battery according to claim 4; and
at least one of a display device, an operation button, an external connection port, a speaker, and a microphone.

10. A vehicle comprising:
the secondary battery according to claim 4; and
at least one of a motor, a brake, and a control circuit.

11. A secondary battery comprising a positive electrode, a negative electrode, and an electrolyte,
wherein the electrolyte comprises the ionic liquid according to claim 2.

12. A secondary battery comprising a positive electrode, a negative electrode, and an electrolyte,
wherein the electrolyte comprises the ionic liquid according to claim 3.

13. The secondary battery according to claim 11,
wherein the positive electrode comprises a positive electrode active material,
wherein the positive electrode active material comprises lithium cobalt oxide to which magnesium, fluorine, aluminum, and nickel are added, and
wherein an XRD pattern at least has a diffraction peak at 2θ=19.30±0.20° and 2θ=45.55±0.10° when the positive electrode is analyzed by powder X-ray diffraction with CuKα₁ radiation in an argon atmosphere in the following manner: the positive electrode and a counter electrode of a lithium metal are used, a mixture in which 2 wt % of vinylene carbonate is added to lithium hexafluorophosphate, ethylene carbonate, and diethyl carbonate is used as the electrolyte, constant current charge with a current value of 0.5 C (note that 1 C=137 mA/g) is performed to a voltage of 4.6 V in an environment at 25° C., and then constant voltage charge is performed until the current value becomes 0.01 C.

14. The secondary battery according to claim 12,
wherein the positive electrode comprises a positive electrode active material,
wherein the positive electrode active material comprises lithium cobalt oxide to which magnesium, fluorine, aluminum, and nickel are added, and
wherein an XRD pattern at least has a diffraction peak at 2θ=19.30±0.20° and 2θ=45.55±0.10° when the positive electrode is analyzed by powder X-ray diffraction with CuKα₁ radiation in an argon atmosphere in the following manner: the positive electrode and a counter electrode of a lithium metal are used, a mixture in which 2 wt % of vinylene carbonate is added to lithium hexafluorophosphate, ethylene carbonate, and diethyl carbonate is used as the electrolyte, constant current charge with a current value of 0.5 C (note that 1 C=137 mA/g) is performed to a voltage of 4.6 V in an environment at 25° C., and then constant voltage charge is performed until the current value becomes 0.01 C.

15. An electronic device comprising:
the secondary battery according to claim 11; and
at least one of a display device, an operation button, an external connection port, a speaker, and a microphone.

16. An electronic device comprising:
the secondary battery according to claim 12; and
at least one of a display device, an operation button, an external connection port, a speaker, and a microphone.

17. A vehicle comprising:
the secondary battery according to claim 11; and
at least one of a motor, a brake, and a control circuit.

18. A vehicle comprising:
the secondary battery according to claim 12; and
at least one of a motor, a brake, and a control circuit.
